(19) United States
(12) Patent Application Publication
(10) Pub. No.: US 2022/0090209 A1
(43) Pub. Date: Mar. 24, 2022

(54) RETROTRANSPOSON BIOMARKERS

(71) Applicants: VIB VZW, Gent (BE); KATHOLIEKE UNIVERSITEIT LEUVEN, K.U.LEUVEN R&D, Leuven (BE)

(72) Inventors: Diether Lambrechts, Kessel-Lo (BE); Jieyi Xiong, Heverlee (BE)

(21) Appl. No.: 17/430,873

(22) PCT Filed: Feb. 13, 2020
(86) PCT No.: PCT/EP2020/053792

§ 371 Date: Aug. 13, 2021

(30) Foreign Application Priority Data
Feb. 14, 2019 (EP) 19157160.3

(51) Int. Cl. C12Q 1/6886; G01N 33/50

(52) U.S. Cl. CPC C12Q 1/6886; C12Q 2600/158; C12Q 2600/106; G01N 33/5091

(57) ABSTRACT

The invention relates to methods of tumor analysis relying on the detection of expression of or of changes in the expression levels of specific retrotransposons. Such methods find application in, amongst other, predicting the response of a tumor to immunotherapy or to immunogenic therapy, and in following up such responses. The expression levels of specific retrotransposons can thus be used in determining which patients are most likely to respond to immunotherapy or immunogenic therapy. Corresponding diagnostic kits are likewise part of the invention.

### CROSS-REFERENCE TO RELATED APPLICATIONS

This application is a national phase entry under 35 U.S.C. § 371 of International Patent Application PCT/EP2020/053792, filed Feb. 13, 2020, designating the United States of America and published in English as International Patent Publication WO 2020/165361 on Aug. 20, 2020, which claims the benefit under Article 8 of the Patent Cooperation Treaty to European Patent Application Serial No. 19157160.3, filed Feb. 14, 2019, the entireties of which are

### FIELD OF THE INVENTION

The invention relates to methods of tumor analysis relying on the detection of expression of or of changes in the expression levels of specific retrotransposons. Such methods find application in, amongst other, predicting the response of a tumor to immunotherapy or to immunogenic therapy, and in following up such responses. The expression levels of specific retrotransposons can thus be used in determining which patients are most likely to respond to immunotherapy or immunogenic therapy.

Corresponding diagnostic kits are likewise part of the invention.

### BACKGROUND

As of its coming to existence, a tumor is creating and/or forced to create its own specific “ecosystem” within the surrounding healthy tissue. Many factors and processes are decisive over whether or not the single tumor cell will be able to create, and support, its ecosystem and, therewith, growth. In an attempt to create a simplifying overview, Blank et al. 2016 (Science 352: 658-660) designed a visually appealing “cancer immunogram” in which currently known factors and processes influencing tumor growth/survival are grouped in seven classes of parameters. For each individual patient/tumor, the status of the seven classes of parameters can be plotted, the resulting plot giving insight in treatment options. Somewhat similar to the cancer immunogram, Charoentong et al. 2017 (Cell Reports 18:248-262) designed an immunophenogram/immunophenoscore which provides an as yet to be further validated tool for predicting response of a tumor to checkpoint blockade therapy (as the majority of cancer patients do not respond to such therapy). Such tools strongly underscores the need to expand knowledge on the status of a tumor or cancer as this, besides potentially leading to identification of new therapeutic targets, aids in deciding on the optimal (available) treatment for each individual tumor. A potential drawback of such tools is the requirement for immunogenomic analysis of the individual cancer.

Recently, Chiappinelli et al. 2016 (Cell 162:974-986) and Roulois et al. 2016 (Cell 162:961-973) showed that DNMTi's (inhibitors of DNA methyltransferase) lead to activation of endogenous retroviruses (ERVs) in ovarian cancer and colorectal cancer, respectively, a mechanism termed as “viral mimicry”. These findings, including implications on combination of epigenetic and immunotherapy, were discussed by Licht 2016 (Cell 162:938-939), Dear 2016 (NEJM 374:684-685) and Chiappinelli et al. 2016 (Cancer Res 76:1683-1689). Furthermore, retroviral expression was linked to predicting a response to immunotherapy in renal cell carcinoma (Smith et al. 2018, J Clin Invest 128:4804-4820).

### SUMMARY OF THE INVENTION

The invention in on aspect relates to methods of determining prior to or early after start of immunotherapy or of an immunogenic therapy the outcome of the immunotherapy or the immunogenic therapy, or of determining susceptibility to the immunotherapy or the immunogenic therapy of a tumor in a subject, comprising the step of detecting the expression level or of detecting a change in the expression level of at least one retrotransposon in a sample obtained from the subject, and wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein an increased expression level of the retrotransposon in the sample relative to the expression level of the same retrotransposon in a control sample or compared to a standard value, is indicative of a positive outcome of the immunotherapy or the immunogenic therapy or is indicative of susceptibility of the tumor to the immunotherapy or the immunogenic therapy. In particular, the expression level of at least 4 retrotransposons may be analysed and an increased expression level of at least 1 of the at least 4 retrotransposons may be detected relative to the expression level of the same retrotransposons in a control sample or compared to a standard value, wherein the increased expression level of the at least 1 retrotransposon is indicative of a positive outcome of the immunotherapy or the immunogenic therapy or is indicative of susceptibility of the tumor to the immunotherapy or the immunogenic therapy.

In an alternative, the invention relates to methods of determining response to immunotherapy or to immunogenic therapy of a tumor in a subject, comprising the step of detecting the expression level or of detecting a change in the expression level of at least one retrotransposon in a sample obtained from the subject, and wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein a decrease in the expression level of the retrotransposon in the sample relative to the expression level of the same retrotransposon in a sample obtained from the subject prior to immunotherapy or immunogenic therapy or in a sample obtained at an earlier time-point during immunotherapy or immunogenic therapy, is indicative of a positive response of the immunotherapy or the immunogenic therapy. In particular, the expression level of at least 4 retrotransposons may be analysed and a decreased expression level of at least 1 of the at least 4 retrotransposons may be detected relative to the expression level of the same retrotransposons in a sample obtained from the subject prior to immunotherapy or immunogenic therapy or in a sample obtained at an earlier time-point during immunotherapy or immunogenic therapy, wherein the decreased expression level of the at least 1 retrotransposon is indicative of a positive outcome of the immunotherapy or the immunogenic therapy.

Any of the above methods may further include detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES).

In any of the above methods at least one analysis step may be performed by a computer system or via a computer program product.

The invention further relates to an immunotherapeutic or immunogenic agent for use in treating a tumor, for use in inhibiting tumor progression or tumor relapse, or for use in inhibiting tumor metastasis, comprising:
detecting the expression level of at least one retrotransposon in a sample obtained from the subject having the tumor, wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5;
detecting an increased expression level of at least one retrotransposon in the sample compared to the expression level of the same retrotransposon in a control sample or compared to a standard value;
administering a therapeutically effective amount of the immunotherapeutic or immunogenic agent to the subject if an increased expression level of at least one retrotransposon is detected.

In particular, the expression level of at least 4 retrotransposons may be analysed and an increased expression level of at least 1 of the at least 4 retrotransposons may be detected relative to the expression level of the same retrotransposons in a control sample or compared to a standard value, wherein the increased expression level of the at least 1 retrotransposon is indicative for administering a therapeutically effective amount of the immunotherapeutic or immunogenic agent to the subject.

The invention further relates to the use of a panel of retrotransposons in a method according to the invention, wherein the panel is comprising 2 to 62 retrotransposons selected from Table 3 or Table 5.

The invention also relates to kits for use in a method according to the invention, wherein the kit is comprising the tools to detect the expression level of at least one retrotransposon selected from Table 3 or Table 5; in particular such kits may be comprising the tools to detect the expression level of 2 to 62 retrotransposons selected from Table 3 or Table 5. Further in particular, such kits may further include the tools for detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES). Further in particular, such kits may be including the tools for detecting the status of at most 500 markers.

The invention also relates to computer products comprising a computer readable medium storing instructions for operating a computer system to perform at least one analysis step of a method according to the invention.

In any of the above, the tumor may in particularly be melanoma.

### DESCRIPTION TO THE FIGURES

FIGS. 1A-1J Methylation at HIF1β binding sites (FIG. 1A.) Heatmaps of HIF1β binding and DNA methylation for 7,153 regions (identified using a stringent threshold of P<10-15 in MACS) surrounding the HIF1β chromatin immunoprecipitation coupled to high-throughput sequencing (ChIP-seq) peak summit (±5 kb). Heatmaps depict reads per kb per million reads (RPKM) of HIF1β ChIP-seq and of 5mC DNA IP-seq (mDIP), and % DNA methylation as estimated by SeqCapEpi bisulfite sequencing (BS-seq) or whole-genome BS-seq (respectively, SeqCapEpi and WGBS). HIF1β binding was assessed after 16 hours of 0.5% O₂ (hypoxia) and DNA methylation under 21% O₂ (normoxia). (FIG. 1B.) Violin plots of the methylation level inside and outside HIF1β binding peaks, as estimated by SeqCapEpi BS-seq. (FIG. 1C.) Sequencing read depth of HIF1β ChIP and its input, at all RCGTG sequences in MCF7 cells, stratified for methylation at the CG in the core RCGTG sequence. Shown are boxplots for all RCGTG's in the human genome for which >10× coverage was obtained after SeqCapEpi BS-seq, with dark red dots denoting averages. (FIG. 1D.) Venn diagram of 20,613 shared and unique HIF1β binding sites detected across 3 cell lines. Only stringent binding sites (P<10\hat{ }-15) are shown. Binding sites showing intermediate levels of HIF1β ChIP-seq enrichment in 1 or 2 cell lines are unclassified and not shown here (n=445, 2,812 and 887 peaks, detected in SK-MEL-28, RCC4 and MCF7 respectively). (FIG. 1E.) Heatmaps of HIF1β binding (red) and DNA methylation as estimated using SeqCapEpi BS-seq (blue) at regions flanking the HIF1β ChIP-seq peak summit (±5 kb). Top, HIF1β binding peaks shared between the 3 cell lines. Bottom, HIF1β binding peaks unique to each cell line. Heatmaps depict reads per kb per million reads (RPKM) of HIF1β ChIP-seq and % DNA methylation. HIF1β binding was assessed after 16 hours of 0.5% O₂ (hypoxia) and DNA methylation under 21% O₂ (normoxia). (FIG. 1F.) Quantification of the methylation level at HIF1β binding peak summits ±100 bp, for peaks that are shared between or unique to one of the 3 cell lines. (FIG. 1G.) Enrichment of gene expression (observed/expected) upon hypoxia per cell line, for genes associated with HIF1β binding sites (within 50 kb) that are shared between or unique to one of the 3 cell lines, as labelled on the X-axis. (FIG. 1H.) Fraction of HIF1β peaks overlapping with the binding peaks of individual transcription factors (Griffon et al. 2015, Nucl Acids Res 43:e27), or with any of the 11 transcription factors profiled in MCF7 cells (“combined”). (FIGS. 1I-1J) (FIG. 1I.) Overlap between HIF1β binding peaks and other transcription factor binding sites detected in MCF7 cells. Shown are fractions of HIF1β binding peaks shared between cell lines or unique for a cell line. (FIG. 1J.) mRNA expression level of transcription factors in each cell line, as determined using RNA-seq. Transcription factors expressed in all 3 cell lines are highlighted as “shared TFs” with a light grey box.

FIGS. 2A-2F. DNA methylation directly repels HIF1β binding

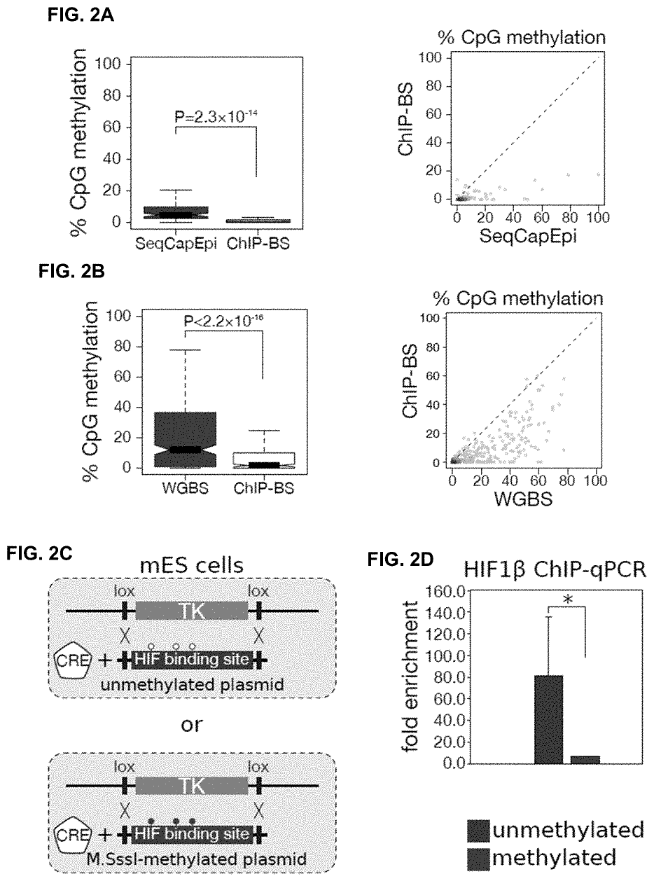

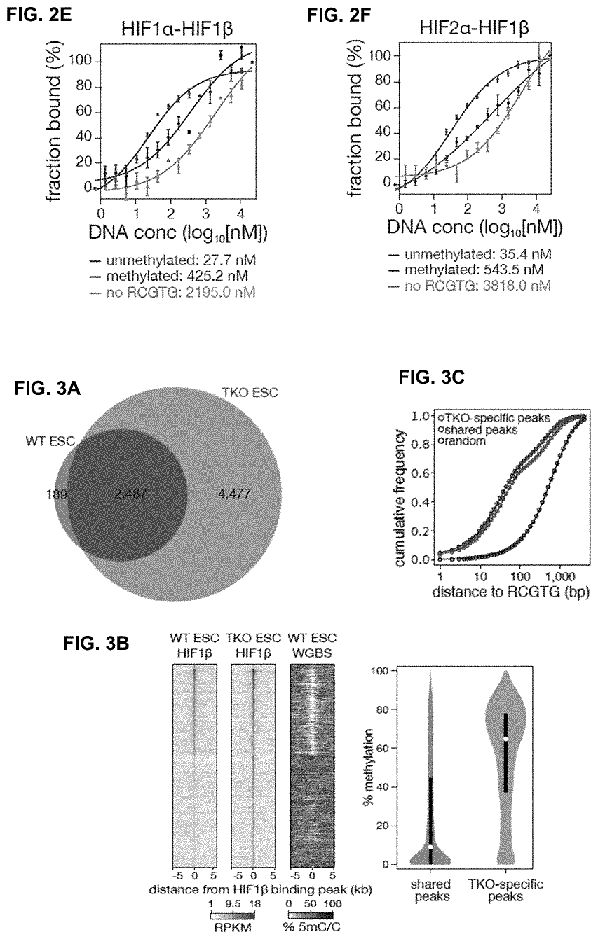

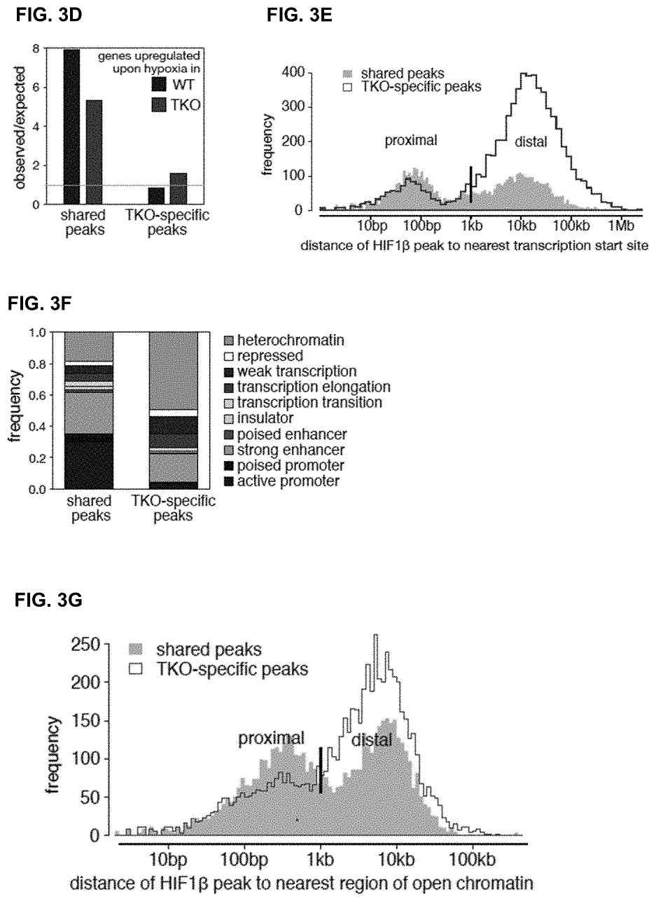

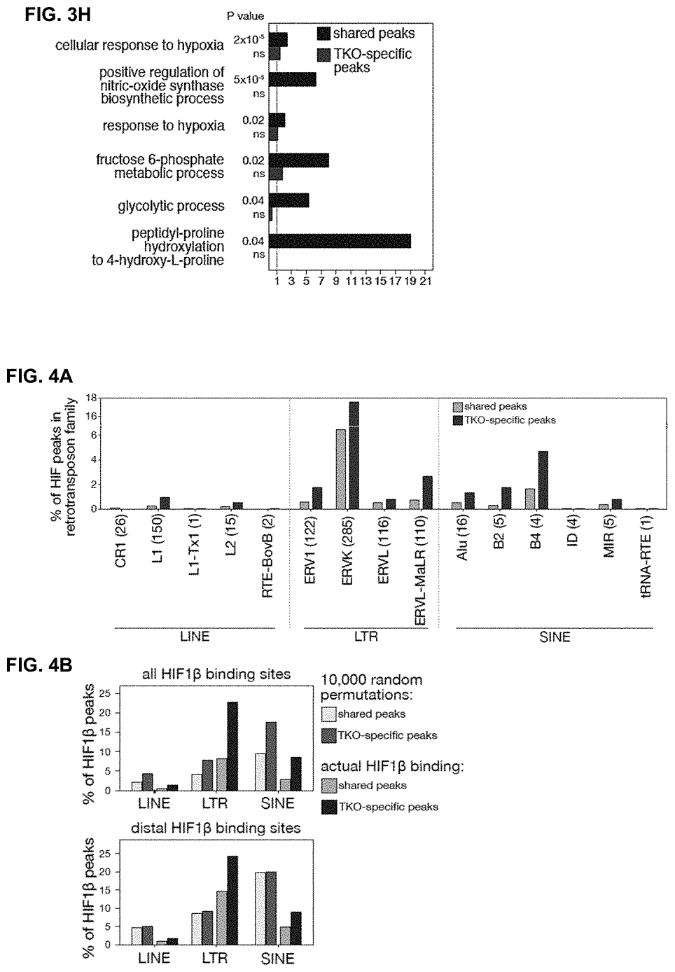

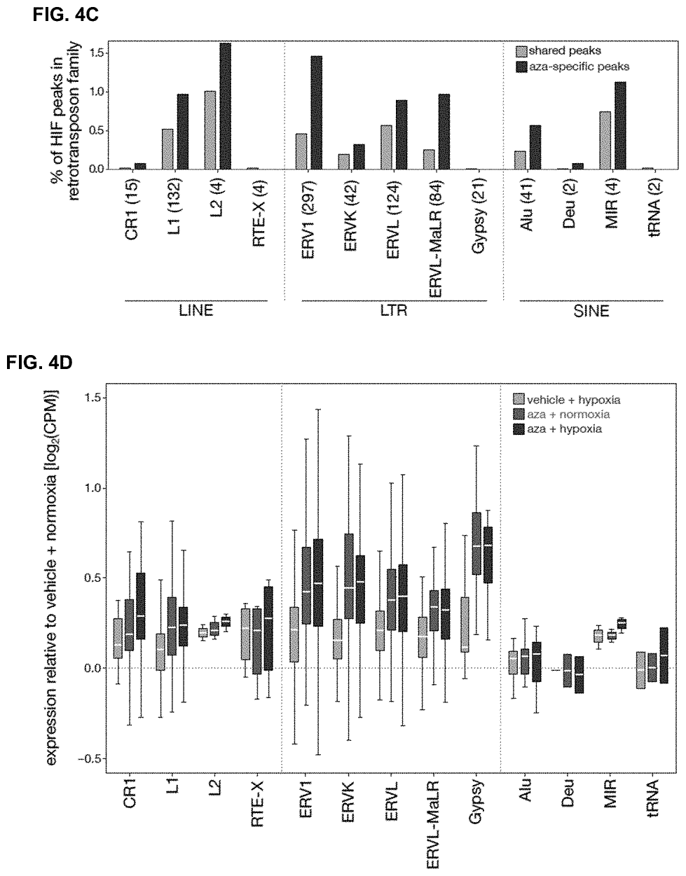

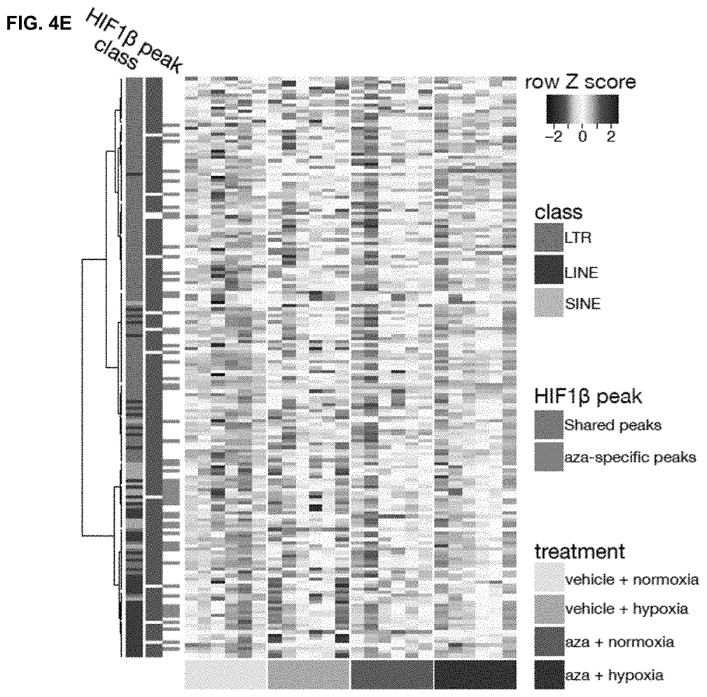

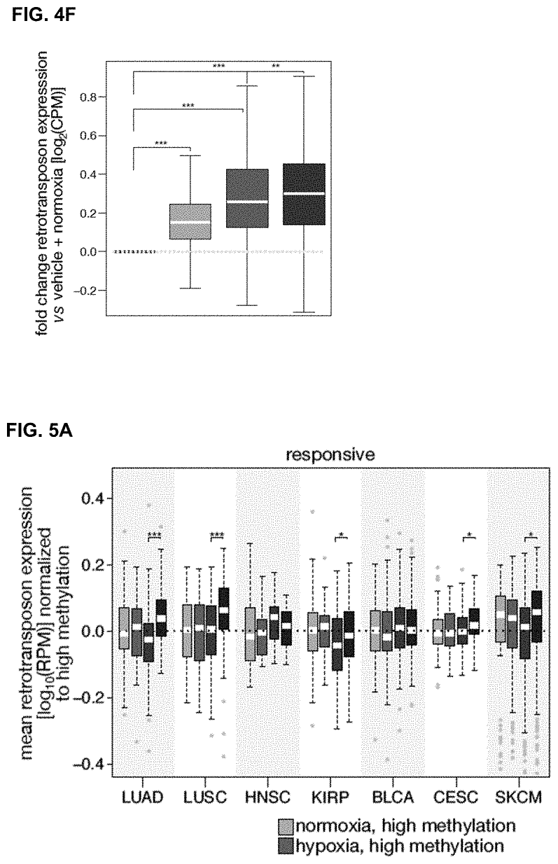

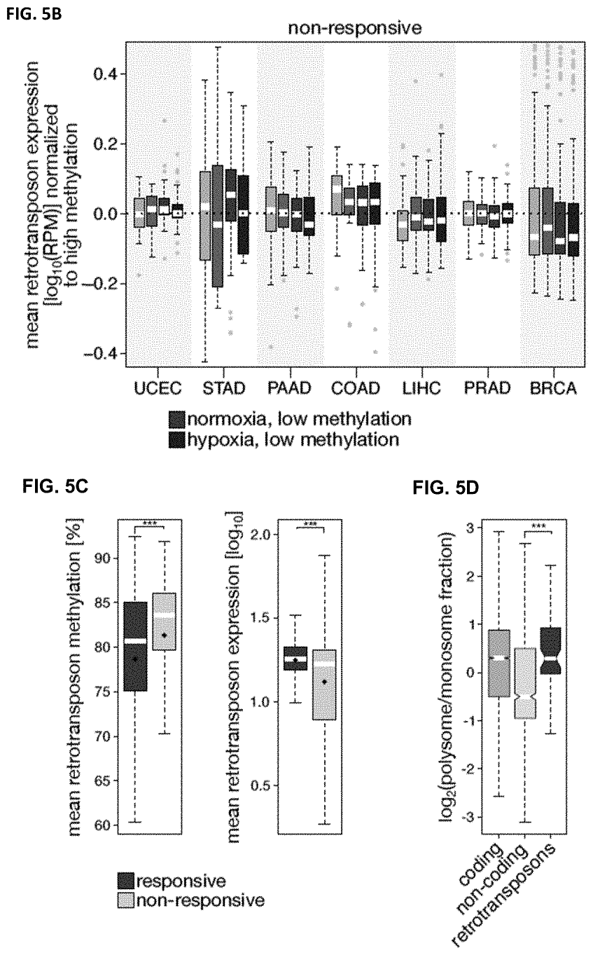

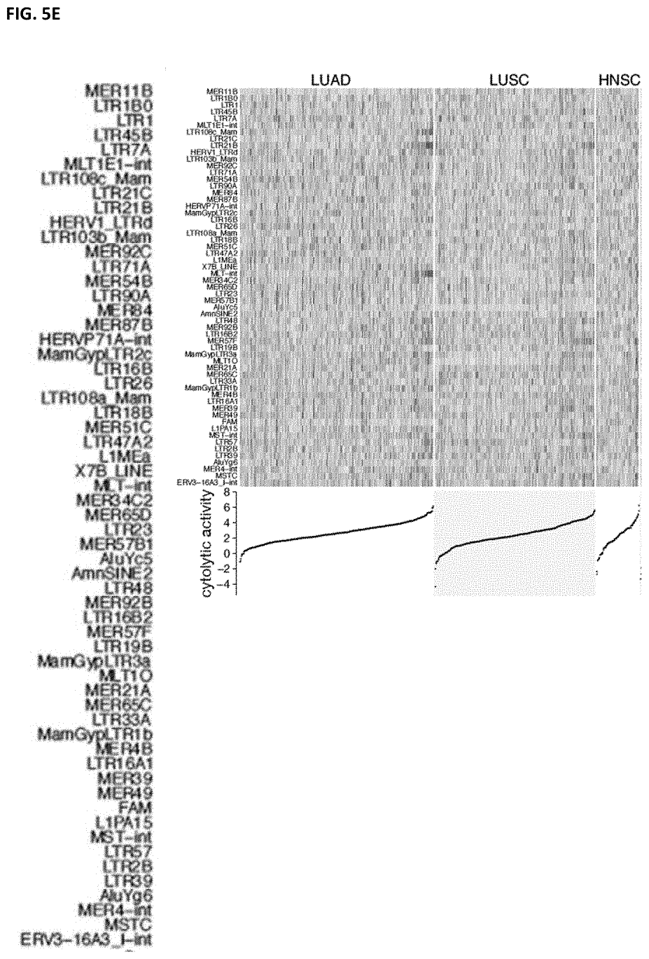

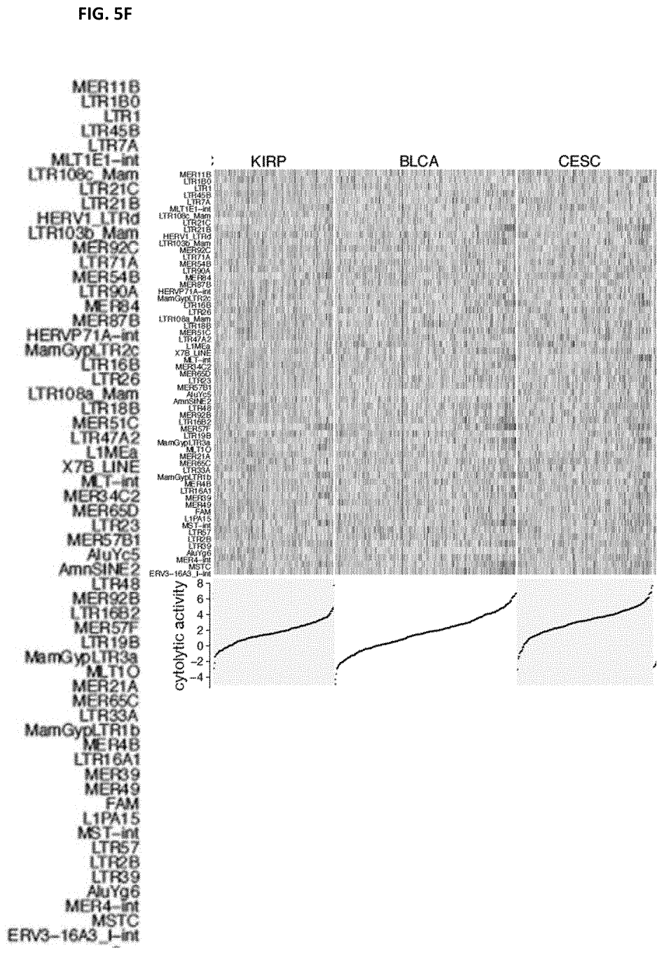

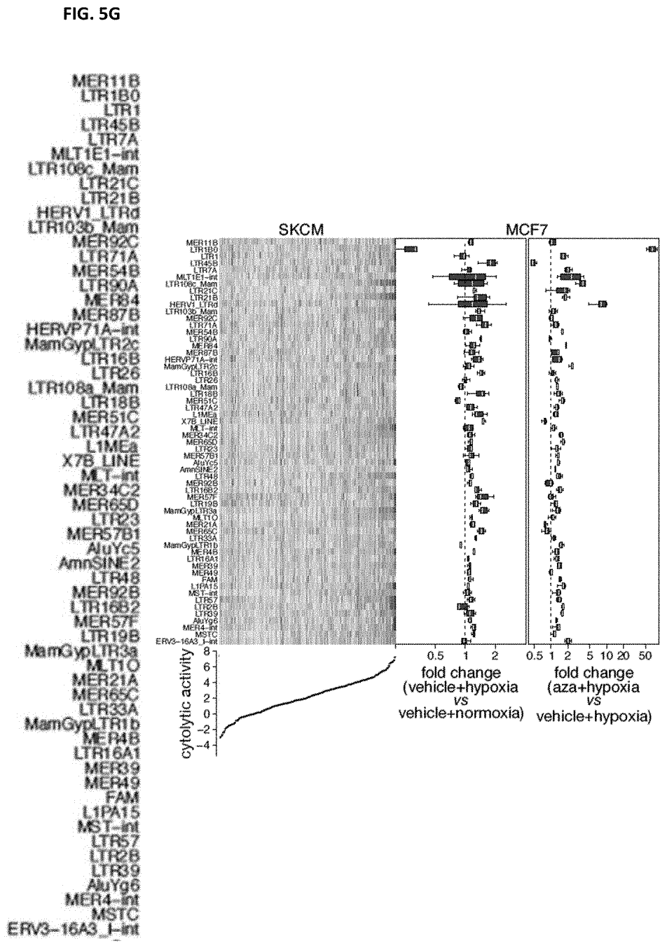

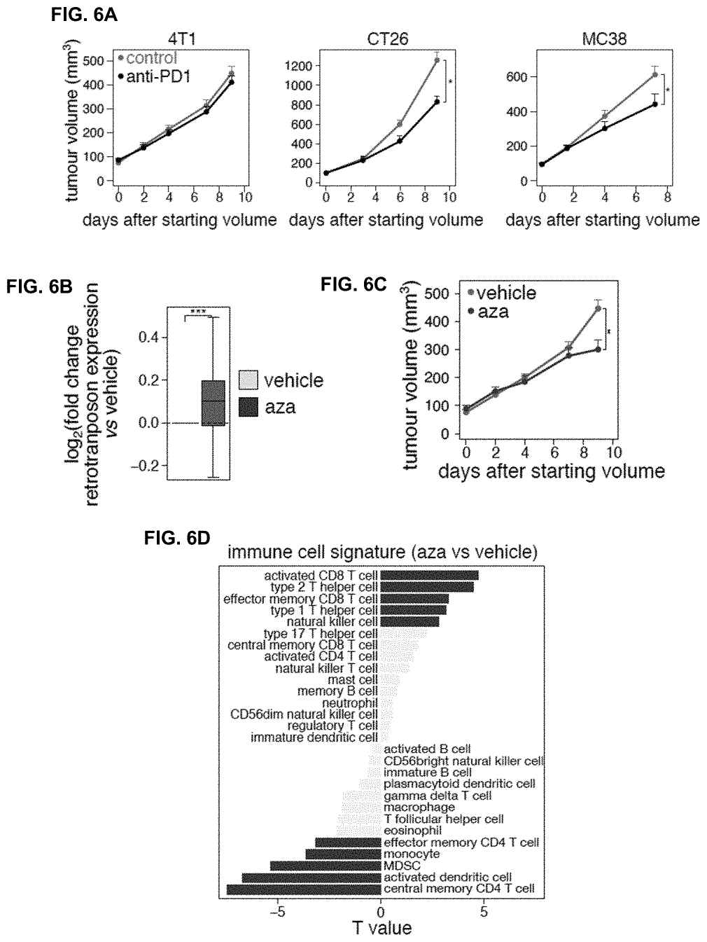

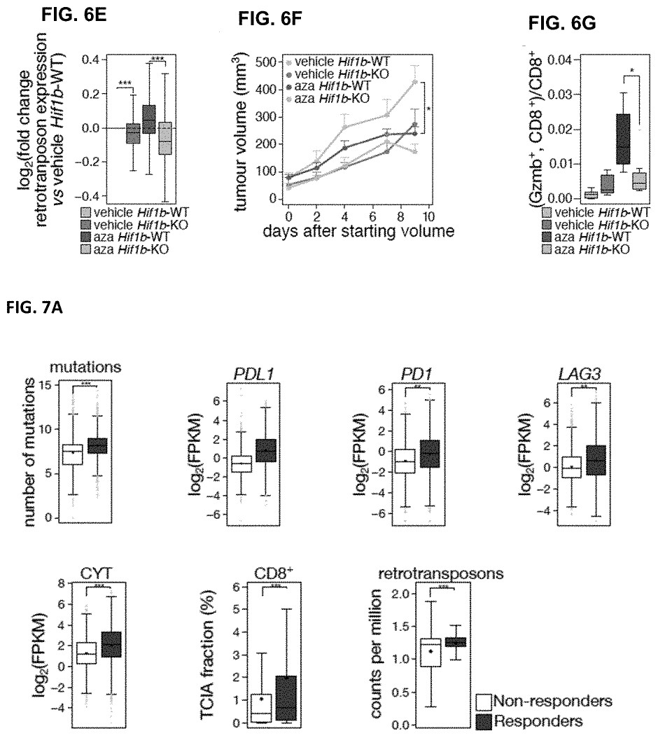

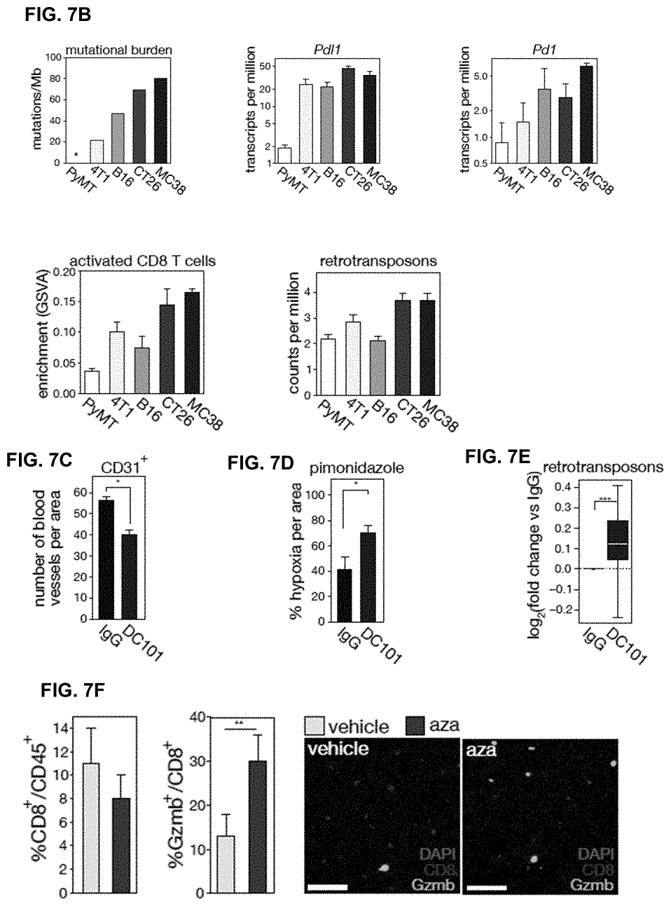

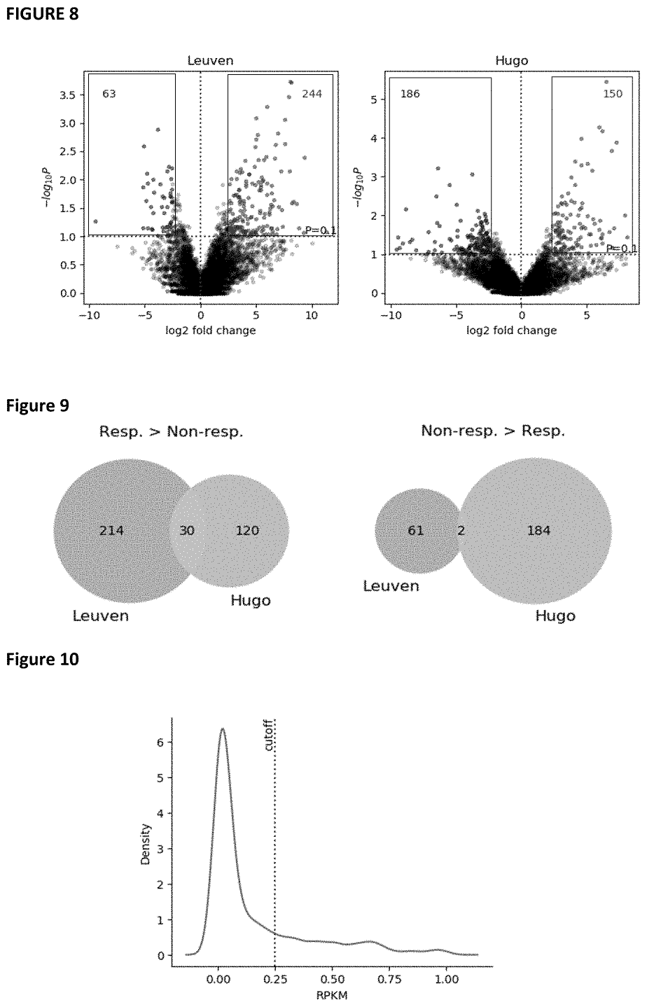

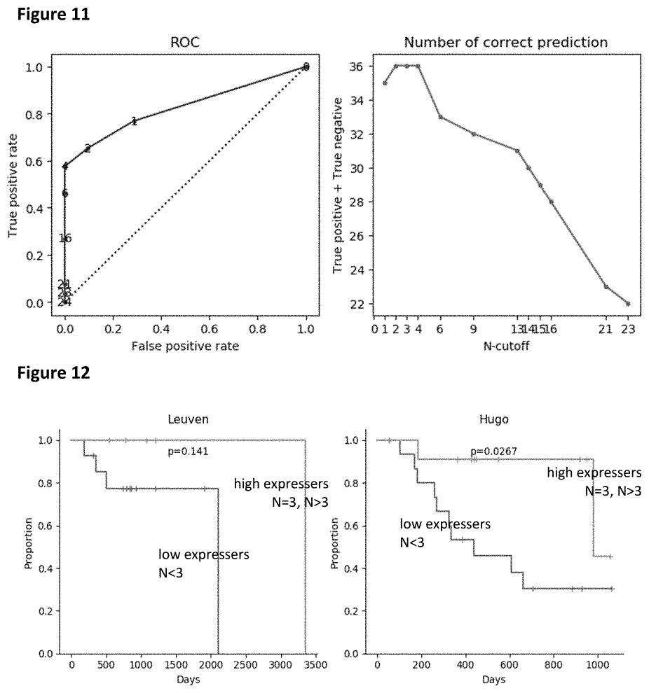

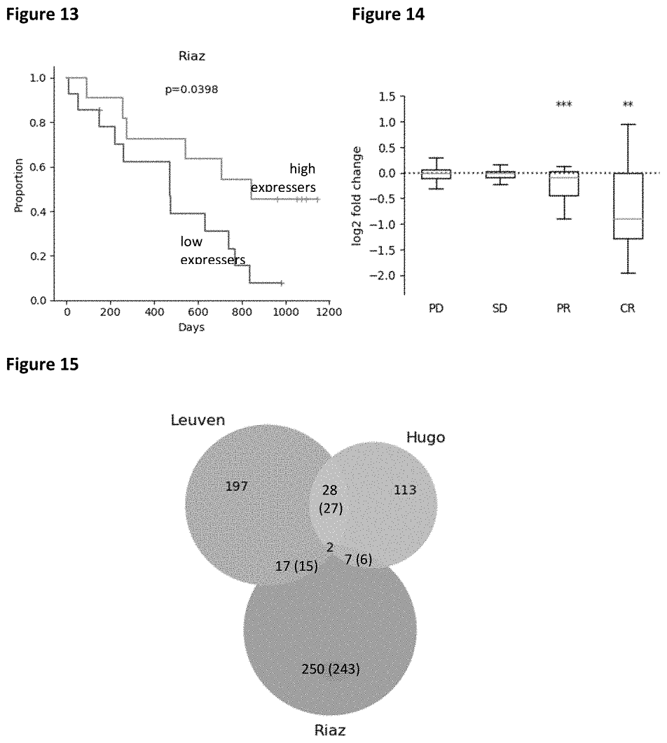

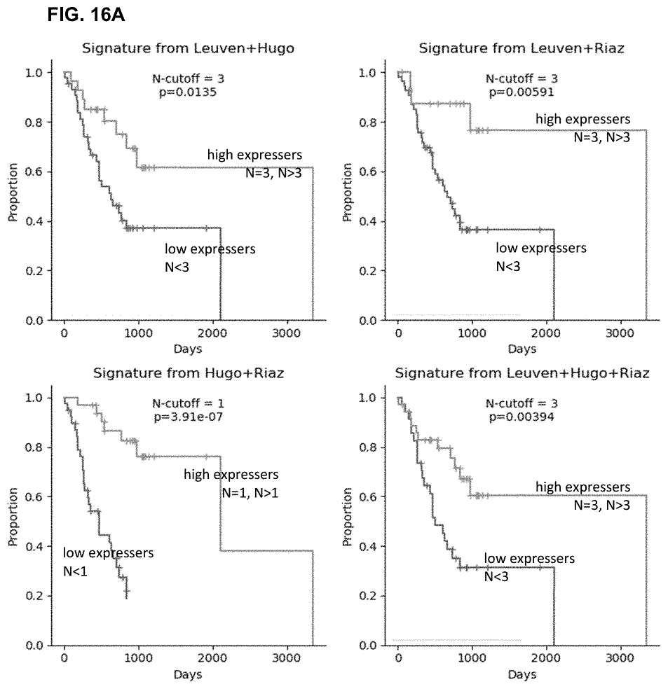

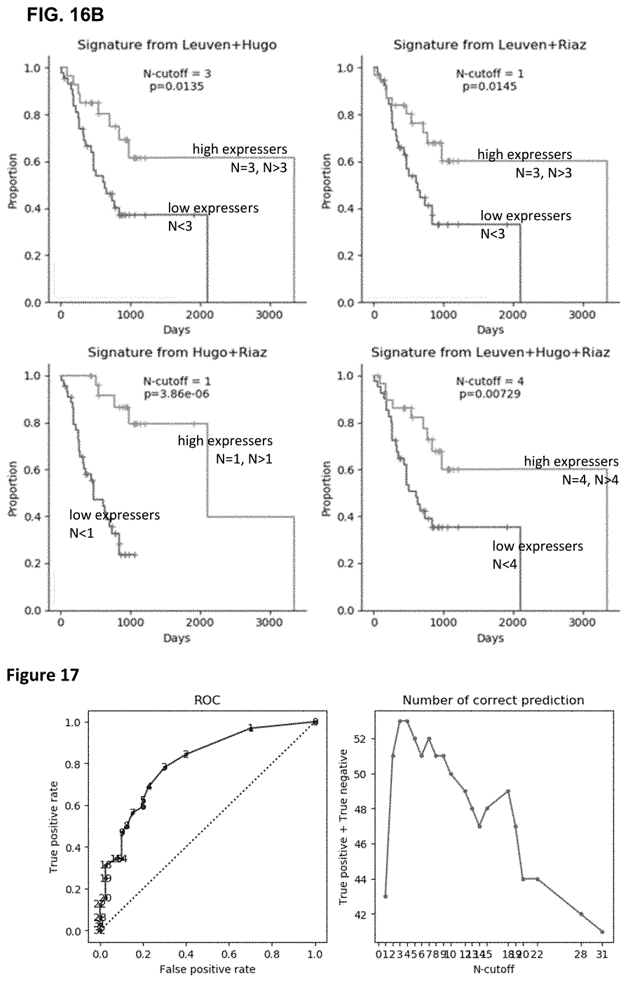

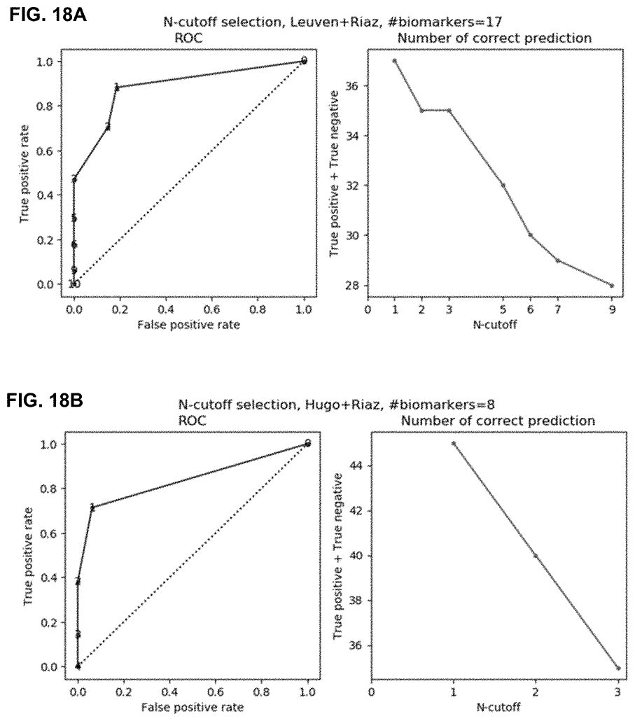

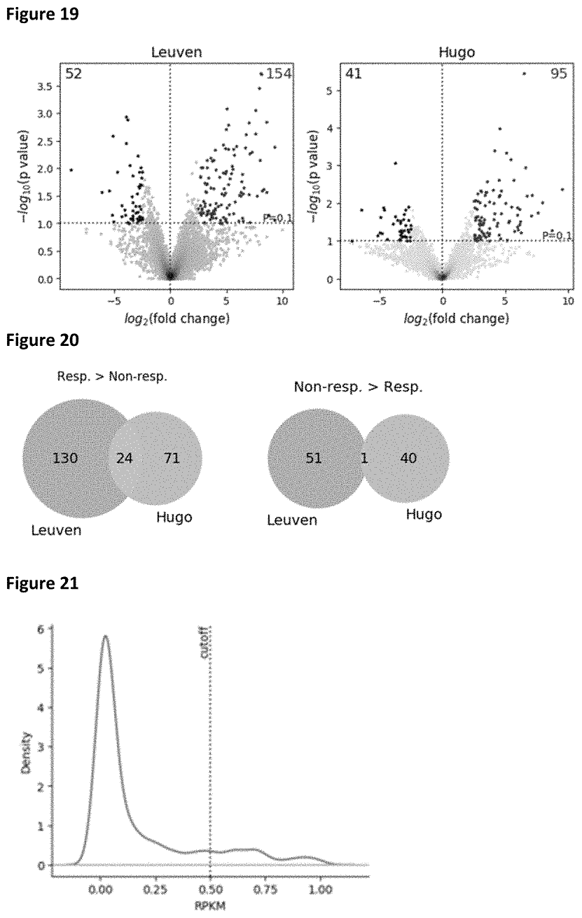

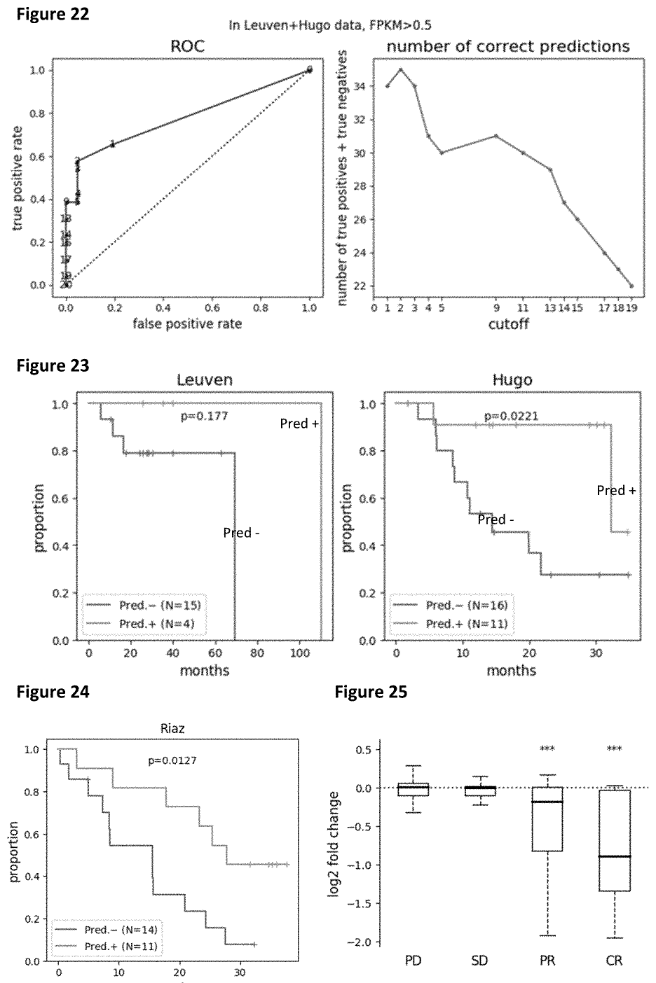

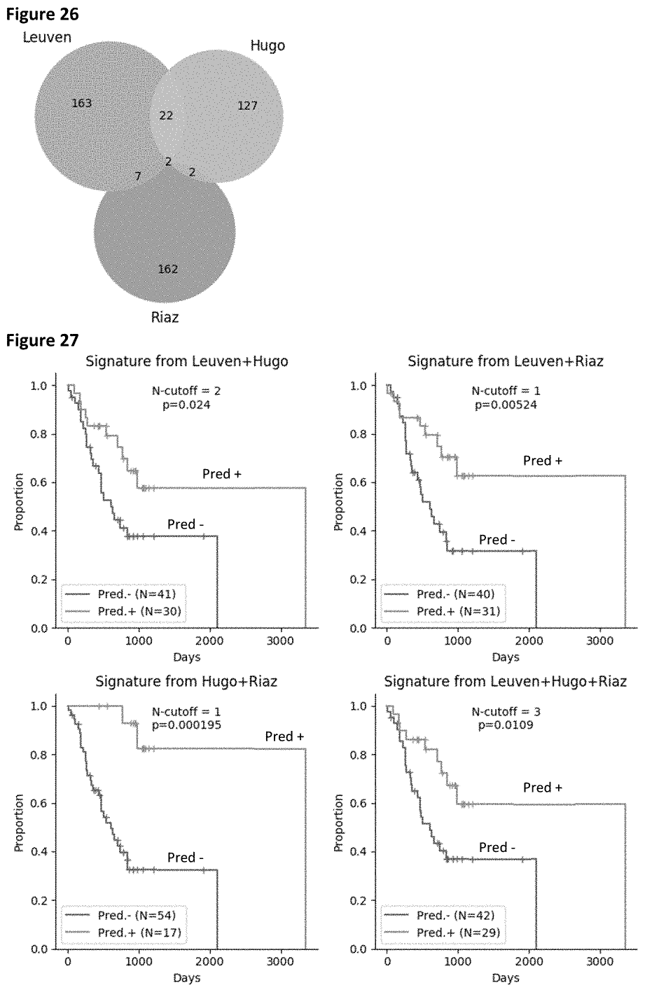

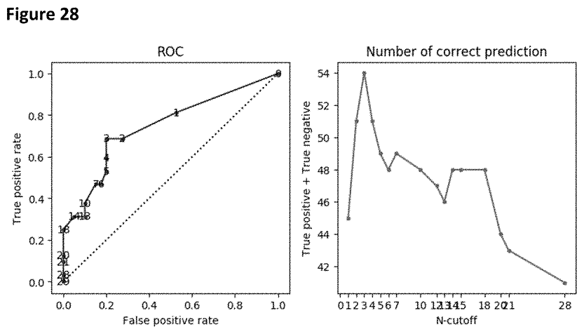

(FIGS. 2A-2B.) Boxplot (left) and scatter plot (right) of methylation levels of HIF1β-bound immunoprecipitated DNA fragments obtained by ChIP-BS-seq (ChIP-BS) compared to input by SeqCapEpi BS-seq (SeqCapEpi) in MCF7 cells (FIG. 2A), or of HIF1β-bound immunoprecipitated DNA fragments obtained by ChIP-BS compared to input by whole genome BS-seq (WGBS) in murine Tet-triple-knockout (Tet-TKO) embryonic stem cells (ESCs) (FIG. 28). The red dotted line in the scatter plot indicates the theoretical value of equal methylation in immunoprecipitated and input DNA. P values by t-test. (FIGS. 2C-2D) Recombination-mediated cassette exchange. (FIG. 2C) A human HIF binding site (chr16: 30,065,212-30,065,711 on hg38) was cloned between 2 L1 lox sites and in vitro methylated or not methylated prior to co-transfection with a CRE recombinase-encoding plasmid into murine ESCs transformed to contain an L1 lox-flanked thymidine kinase (TK). (FIG. 2D) Following successful cassette exchange, these ESCs were incubated in hypoxia (0.5% O₂ for 16 hours) and probed using HIF1β ChIP-qPCR for HIF binding at the differentially methylated cassette. Shown is the fold enrichment over background (n=3 independent ChIP pairs). (FIGS. 2E-2F) microscale thermophoresis-based assessment of sensitivity of HIF1α-HIF1β (FIG. 2E) and HIF2α-HIF1β (FIG. 2F) heteroduplexes to methylation at HIF binding sites in physiological buffer (PBS). RCGTG sequences in the double-stranded DNA oligonucleotides were either absent, methylated or unmethylated at the CpG site. KD values are shown under each graph.

FIGS. 3A-3H. DNA demethylation uncovers new HIF1β binding sites

(FIG. 3A) Overlap of HIF1β peaks detected in murine ESCs (at a threshold of P<10\hat{ }-5) that are wild-type (WT) or triple-knockout (TKO) for Dnmt1, Dnmt3a and Dnmt3b (resp. WT and TKO ESCs). (FIG. 3B) (left) Heatmaps of HIF1β binding (reads per kb per million reads, RPKM) and DNA methylation as determined using WGBS at regions flanking the summit of HIF1β binding peaks (±5 kb) either shared with murine WT or Dnmt-TKO-specific ESCs. (right) % methylation at shared and Dnmt-TKO-specific (TKO-specific) HIF1β binding sites in murine WT ESCs. (FIG. 3C) Cumulative frequency of distance to the nearest RCGTG motif for shared, TKO-specific and randomized HIF1β binding peaks. (FIG. 3D) Observed/expected frequency of upregulated genes associated with shared and TKO-specific HIF1β binding peaks in murine WT and Dnmt-TKO ESCs exposed to 24 hours of hypoxia (0.5% O₂). (FIG. 3E) Distance of shared and TKO-specific HIF1β binding peaks to the nearest transcription start sites (TSS). A bimodal peak was detected indicating proximal and distal binding events. (FIG. 3F) Functional genome annotation using ChromHMM of shared and TKO-specific HIF1β binding peaks in ESCs. (FIG. 3G) Distance of shared and TKO-specific HIF1β binding peaks to open chromatin regions in ESCs. A bimodal peak was detected indicating proximal and distal binding events. (FIG. 3H) Ontology analysis of genes associated with shared and TKO specific HIF1β binding peaks in ESCs.

FIGS. 4A-4F. HIF binds retrotransposons in demethylated genomes

(FIG. 4A) % of shared or Dnmt-TKO-specific (TKO-specific) HIF1β binding peaks in ESCs that overlap retrotransposons, grouped by retrotransposon class and family. Families not bound by HIF1β (Dong-R4, Jockey, Penelope, RTE-X, Gypsy, 5S-Deu-L2, tRNA, tRNA-Deu) or containing unclassifiable subfamilies are not shown. The number of retrotransposon subfamilies involved is indicated in brackets. (FIG. 4B) HIF1β binding sites in LINEs, LTRs and SINEs after 10,000 random permutations and as observed by HIF1β ChIP-seq (actual HIF1β binding) for all sites (upper) and only for distal sites (lower). (FIG. 4C) As in (FIG. 4A), but for HIF1β binding peaks that are shared between vehicle- and aza-treated MCF7 cells, or specific to aza-treated MCF7 cells. (FIG. 4D) Differential expression of 13 retrotransposon families bound by HIF1β. Boxplots show average read counts from RNA-seq analyses of vehicle- and aza-treated MCF7 cells exposed for 24 hours to normoxia or hypoxia (resp. 21% and 0.5% O₂), as determined using RepEnrich (n=6 per condition). Families not bound by HIF1β or containing unclassifiable subfamilies are not shown. (FIG. 4E) Heatmap showing expression of 176 retrotransposon subfamilies bound by HIF1β in hypoxic MCF7 cells exposed to vehicle (DMSO) or aza. Expression was determined using RepEnrich (n=6 per treatment condition). (FIG. 4F) Difference in the distribution of retrotransposon expression, as determined using RepEnrich. Shown is the difference of log₂ read counts from RNA-seq analyses of vehicle- and aza-treated MCF7 cells exposed for 24 hours to hypoxic conditions (0.5% O₂) versus MCF7 cells exposed to normoxic conditions (21% O₂). ***P<0.001; **P<0.01 by paired t-test.

FIGS. 5A-5G. Retrotransposon expression in tumours

(FIGS. 5A-5B) Retrotransposon expression in tumours from the TCGA dataset, as determined using RepEnrich. Per tumour type, tumours were classified as normoxic or hypoxic by clustering analysis using a well-established hypoxia gene signature (see Methods). Median methylation levels at retrotransposons were calculated based on 556 probes from the 450K BeadChip that overlap intergenic retrotransposons. Within hypoxic and normoxic clusters, each tumour was classified as having high methylation if the median of the 556 beta values was higher than the median of the cluster. Average of retrotransposon expression (log₁₀ (RPM)) was calculated and normalized to the high methylation group within hypoxic and normoxic clusters separately. *P<0.05; **P<0.01; ***P<0.001 by t-test. (FIG. 5C) Methylation level at retrotransposons (left) and retrotransposon expression (right) in tumours profiled in TCGA, stratified into tumour types that are responsive (n=2,280) or non-responsive (n=2,214) to checkpoint immunotherapy. ***P<0.001 by t-test. (FIG. 5D) Ribosome binding analysis in MCF7 cells. Shown is the polysome over monosome ratio of protein coding genes, non-coding genes and of the 59 retrotransposons associated with cytolytic activity in the TCGA data. ***P<0.001 by t-test. (FIGS. 5E-5G) Heatmap showing the expression (Z-score, blue to red) of the 59 retrotransposons associated with cytolytic activity in tumours responsive to immunotherapy from the TCGA dataset. The box plot on the right depicts the log-fold change in expression of the same 59 retrotransposons in hypoxic versus normoxic MCF7 cells (24 hours, 0.5% O₂), and of MCF7 cells after 4-day exposure to aza versus vehicle-treated hypoxic MCF7 cells. At the bottom, cytolytic activity of each TCGA sample is depicted. LUSC, lung squamous cell carcinoma; LUAD; lung adenocarcinoma; HNSC, head and neck squamous cell carcinoma; KIRP, kidney renal papillary cell carcinoma; BLCA, bladder urothelial carcinoma; CESC, cervical squamous cell carcinoma and endocervical adenocarcinoma; SKCM, skin cutaneous melanoma; COAD, colon adenocarcinoma; UCEC, uterine corpus endometrial carcinoma; STAD, stomach adenocarcinoma; PAAD, pancreatic adenocarcinoma; LIHC, liver hepatocellular carcinoma; PRAD, prostate adenocarcinoma; BRCA, breast invasive carcinoma.

FIGS. 6A-6G. Aza treatment increases tumour immunogenicity HIF-dependently

(FIG. 6A) Growth of tumours generated by grafting mice subcutaneously with CT26 and MC38, or orthotopically with 4T1 cells. Mice were treated with anti-PD1 or control IgG antibody (see methods). Data represent estimated mean and s.e.m. from 3 independent experiments, each with at least n=6 per group. * P<0.05 by repeated measurement analysis. (FIGS. 6B-6C) Boxplot showing the expression of 226 retrotransposon subfamilies (bound by HIF1β in murine WT and Dnmt-TKO ESCs) (FIG. 6B) and tumour growth (FIG. 6C) in vehicle- and aza-treated 4T1 tumours. Expression was determined using RepEnrich (at least n=4 per treatment condition). Difference in the distribution of retrotransposon expression is expressed as log₂-fold change of counts per million over control 4T1 tumours. ***P<0.001 by paired t-test. Tumour growth data represent estimated mean and s.e.m. from independent experiments, each with at least n=6 mice per group. * P<0.05 by repeated measurement analysis. (FIG. 6D) Association between aza treatment and immune cell infiltration estimates in 4T1 tumours from mice treated with either aza or PBS, as calculated by GSVA on PanCancer immune metagenes (Charoentong et al. 2017, Cell Rep 18:248-262) and visualized by their T value (at least 4 mice per treatment condition were sequenced). Statistically significant associations are depicted in dark grey (P<0.05). (FIG. 6E) Boxplot showing the expression of 226 retrotransposon subfamilies (bound by HIF1β in murine WT and Dnmt-TKO ESCs) in 4T1 tumours WT (Hif1b-WT) or KO (Hif1b-KO) for Hif1b implanted in mice treated with vehicle or aza (see methods, at least 6 tumours per treatment condition were sequenced). Expression was determined using RepEnrich. Difference in the distribution of retrotransposon expression is expressed as log₂-fold change of counts per million over vehicle-treated WT 4T1 tumours. ***P<0.001 by paired t-test. (FIG. 6F) Growth of tumours generated by grafting mice orthotopically with Hif1b-WT 4T1 cells (4T1Hif1b-scr) or Hif1b-KO 4T1 cells (4T1Hif1b-KO). Mice were treated with aza or vehicle (PBS) (see methods). Data represent mean and s.e.m. from independent experiments each with at least n=6 mice per group. *P<0.05 by t-test. (FIG. 6G) Quantification of CD8+ and granzyme b (Gzmb)+ cells, depicted as percentage of CD8+ cells, from tumours generated by grafting mice orthotopically with Hif1b-WT 4T1 cells (4T1Hif1b-scr) or Hif1b-KO 4T1 cells (4T1Hif1b-KO) and treated with aza or vehicle (PBS) (n=6 per group; see methods). *P<0.05 by t-test.

FIGS. 7A-7F. Aza treatment increases immunogenicity

(FIG. 7A) Immunogenicity estimates in TCGA tumours responsive or non-responsive to immunotherapy34. Shown are number of somatic mutations extracted from the TCGA database, mRNA expression of PDL1, PD1 and LAG3 as log 2 RPKM, cytolytic activity (CYT) defined as the log 2-average (geometric mean) of GZMA and PRF1 expression in RPKM, cell fraction for CD8+ T cells estimated with respect to the total cells in the sample as defined by quanTIseq (The Cancer Immunome Atlas, TCIA58), and retrotransposon expression as log 10 counts per million. ***P<0.001; **P<0.01 by t-test. (FIG. 7B) Immunogenicity estimates in spontaneous (PyMT) or cell line grafted (4T1, B16, CT26 and MC38) murine tumour models. Shown are mutational burden expressed as the number of mutations per Mb of expressed coding DNA sequence (CDS) (* PyMT having no mutator phenotype61), mRNA expression (transcripts per million, TPM) of Pd11 and Pd1 immune checkpoint molecules, activated CD8 T-cell enrichment estimated through gene set variance analysis (GSVA) of immune metagenes58 and retrotransposon expression (average counts per million+s.e.m.). (FIGS. 7C-7E) Quantification of the number of blood vessels (CD31 staining), percentage of hypoxia (pimonidazole staining) and retrotransposon expression (as log 2-fold change of counts per million over IgG-treated tumours) in 4T1 tumours from mice injected with DC101 or control IgG (with at least 4 mice per treatment condition, see methods). (FIG. 7F) Quantification of CD8+ and granzyme b (Gzmb)+ cells, depicted as percentages of CD45+ cells and CD8+ cells, from vehicle- or aza-treated 4T1 tumours bearing mice and representative immunofluorescence images (scale 50 μm). **P<0.01 by t-test.

FIG. 8. Volcano plots for DESeq test between response and non-response patient groups

Boxed dots are retrotransposons under P<0.1 and with an absolute value of log 2 fold change >2.5. Within each patient cohort, right boxed dots are retrotransposons highly expressed in the response group and left boxed dots are retrotransposons highly expressed in the non-response group. The numbers of retrotransposons in each box is indicated in the top corners; these numbers were also used to draw FIG. 9.

FIG. 9. Venn plots show the differently expressed retrotransposons shared between cohorts

The retrotransposons highly expressed in the response group and non-response group are shown on the left panel and right panel, respectively. The shared 30 retrotransposons in the left panel were used as biomarkers (and sometimes referred to as “Sig30” hereinafter). Details on these 30 retrotransposons is listed in Table 3. When omitting retrotransposons located on the Y chromosome, the numbers of retrotransposons with higher expression in responders vs. non-responders are 212 (instead of 214), 29 (instead of 30) and 119 (instead of 120). The 29 retrotransposons common to the Leuven and Hugo cohorts are referred to as “Sig29” hereinafter (same as Sig30 from which the Y chromosome-located retrotransposon is removed). The biomarkers constituting the Sig30/Sig29 biomarker panels are derivable from Table 3; and see further).

FIG. 10. RPKM density plot of the 30 biomarkers (Sig30) in Leuven and Hugo cohorts

Data with zero-read or RPKM>1 were excluded.

FIG. 11. ROC curves (left) and the number of correct prediction (right) on different N-cutoff in Leuven and Hugo cohorts. The highest prediction correctness is reached when N-cutoff is 3, 4 or 5. Results are basically unchanged when repeating with the Sig29 retrotransposon signature.

FIG. 12. Overall survival analysis on modelling cohorts

The patients were predicted as either responsive (high expressers, expressing N≥3 retrotransposons) or non-responsive (low expressers, expressing N<3 retrotransposons) to immune checkpoint therapy using the 30-retrotransposon-signature (Sig30). The p-values were tested using “survdiff” function of R-survival package. Number of high and low expressers in Leuven cohort: 5 and 14, respectively. Number of high and low expressers in Hugo cohort: 11 and 16, respectively.

FIG. 13. Overall survival analysis on Riaz cohort

The patients were predicted as either responsive (high expressers, expressing N≥3 retrotransposons) or non-responsive (low expressers, expressing N<3 retrotransposons) to immune checkpoint therapy using the 30-retrotransposon-signature. The p-value was tested using “survdiff” function of R-survival package. Number of high and low expressers in Riaz cohort: 11 and 14, respectively.

FIG. 14. Biomarkers expression changes after treatment in Riaz cohort

Log 2-transferred fold changes of each paired pre-treatment/on-treatment samples were used. Wilcoxon signed rank tests were used for the significance. **-p<0.01, ***-p<0.001.

FIG. 15. Venn plots show the differently expressed retrotransposons in three cohorts

Only retrotransposons with P<0.1, log 2 fold change>2.5 and highly expressed in the response group were shown. This enabled delineation of 19 (or 17) and 9 (or 8) differentially expressed transcripts (Sig19 (or Sig17), and Sig9 (or Sig29), respectively) shared between the Leuven and Riaz cohorts, and between the Hugo and Riaz cohorts, respectively. The Sig19 (or Sig17) and Sig9 (or Sig8) biomarker panels are derivable from Table 3 (and see further). The 54 (or 50) differentially expressed transcripts shared between any two of three cohorts were regarded as an extended biomarkers, and constitute the Sig54 (or Sig50) signature or panel (see Table 3). Numbers between brackets relate to the changes in number of retrotransposons upon omission of the Y chromosome-located retrotransposons.

FIGS. 16A-16B. Overall survival analysis based on the signatures detected from two or three cohorts

(A) (FIG. 16A) For all the four tests, the biomarker expression cutoffs are RPKM=0.25. The N-cutoffs were detected in the modelling cohorts in similar methods as FIG. 11. For the OS analysis but not for the signature discovery, all the patients from three cohorts were merged, and patients were predicted as either responsive (high expressers: expressing N≥3 retrotransposons, except for Hugo+Riaz where N=1) or non-responsive (low expressers, expressing N<3 retrotransposons, except for Hugo+Riaz) to immune checkpoint therapy using the 30-retrotransposon-signature (panel I, top left; panel “signature from Leuven+Hugo”; “Sig30”), using the 19-retrotransposon-signature (panel II, top right; panel “signature from Leuven+Riaz”; “Sig19”), using the 9-retrotransposon-signature (panel III, bottom left; panel “signature from Hugo+Riaz”; “Sig9”), or using the 54-retrotransposon-signature (panel IV, bottom right; panel “signature from Leuven+Hugo+Riaz”; “Sig54”). The p-values were tested using “survdiff” function of R-survival package. Details on these different retrotransposons can be retrieved from Table 3. Number of high and low expressers in cohorts analyzed with Sig30: 27 and 44, respectively. Number of high and low expressers in cohorts analyzed with Sig19: 17 and 54, respectively. Number of high and low expressers in cohorts analyzed with Sig9: 32 and 39, respectively. Number of high and low expressers in cohorts analyzed with Sig54: 36 and 35, respectively.

(FIG. 16B) Same as (FIG. 16A), but omitting the Y chromosome-located retrotransposons. Number of high and low expressers in cohorts analyzed with Sig29: 27 and 44, respectively. Number of high and low expressers in cohorts analyzed with Sig17: 32 and 39, respectively. Number of high and low expressers in cohorts analyzed with Sig8: 26 and 45, respectively. Number of high and low expressers in cohorts analyzed with Sig50: 30 and 41, respectively.

FIG. 17. ROC curves (left) and the number of correct prediction (right) on different N-cutoff for the extended 54 biomarkers in merged three cohorts

The highest prediction correctness is reached when N-cutoff is 3 or 4. Results are basically unchanged when repeating with the Sig50 retrotransposon signature (omission of Y chromosome located retrotransposons).

FIGS. 18A-18B. ROC curves (left) and the number of for the biomarkers in merged sample cohorts

(FIG. 18A) Leuven and Riaz cohorts merged, with omission of the Y chromosome located retrotransposons.

(FIG. 18B) Hugo and Riaz cohorts merged, with omission of the Y chromosome located retrotransposons.

FIG. 19. Volcano plots for DESeq test between response and non-response patient groups. Black dots represent the differentially expressed transcripts under P<0.1 and the absolute value of log 2 fold change >2.5. Black dots representing differentially expressed transcripts highly expressed in the response group are in the right-hand parts of the panels (positive log 2 (fold change)) and black dots representing differentially expressed transcripts highly expressed in the non-response group are in the left-hand parts of the panels (negative log 2 (fold change)). The respective numbers of differentially expressed transcripts are indicated in the right and left corners of each panel.

FIG. 20. Venn plots show the differently expressed transcripts shared between patient cohorts. The differentially expressed transcripts highly expressed in the response group and non-response group are shown on the left panel and right panel, respectively. The 24 shared differentially expressed transcripts in the left panel were used as the biomarkers constituting the Sig24 biomarker panel (derivable from Table 5; and see further).

FIG. 21. RPKM density plot of the 24 biomarkers in Leuven and Hugo cohorts. Data with zero-read or RPKM>1 were excluded.

FIG. 22. ROC curves (left) and the number of correct prediction (right) on different N-cutoff in Leuven and Hugo cohorts. The highest prediction correctness is reached when N-cutoff is 2.

FIG. 23. Overall survival analysis on modeling cohorts. The patients were predicted as either responding (Pred.+) or non-responding (Pred.−) using the Sig24 signature. The p-values were tested using “survdiff” function of R-survival package.

FIG. 24. Overall survival analysis of independent cohort (Riaz). The patients were predicted as either responding (Pred.+) or non-responding (Pred.−) using the Sig24 signature. The p-value was tested using “survdiff” function of R-survival package.

FIG. 25. Biomarkers expression changes after treatment in Riaz cohort. Log 2-transferred fold changes in expression of the markers of the Sig24 signature for each paired pre-treatment/on-treatment samples. Wilcoxon signed rank tests were used for the significance. ***-p<0.001.

FIG. 26. Venn plots show the differently expressed transcripts in three cohorts. Only differential transcripts with P<0.1, log 2 fold change>2.5 and highly expressed in the response group were shown. This enabled delineation of 9 and 4 differentially expressed transcripts (Sig9b and Sig4, respectively) shared between the Leuven and Riaz cohorts, and between the Hugo and Riaz cohorts, respectively. The Sig9b and Sig4 biomarker panels are derivable from Table 5 (and see further). The 33 differentially expressed transcripts shared between any two of three cohorts were regarded as an extended biomarkers, and constitute the Sig33 signature or panel (see Table 5).

FIG. 27. Overall survival analysis based on the signatures detected from two or three cohorts. For all the four tests, the biomarker expression cutoffs are RPKM=0.5. The N-cutoffs were detected in the modeling cohorts in similar methods as FIG. 23. For the overall survival (OS) analysis, the patients from all three cohorts were merged, and patients were predicted as either responding (Pred.+) or non-responding (Pred.−) using the Sig24 signature (panel I, top left), the Sig9b signature (panel II, top right), the Sig4 signature (panel III, bottom left), and the Sig33 signature (panel IV, bottom right). The p-values were tested using “survdiff” function of R-survival package.

FIG. 28. ROC curves (left) and the number of correct prediction (right) on different N-cutoff for the extended 33 biomarkers (Sig33) in merged three cohorts. The highest prediction correctness is reached when N-cutoff is 3.

### DETAILED DESCRIPTION TO THE INVENTION

The work leading to the current invention initially focused on the effect of DNA methylation on genome wide binding of hypoxia-inducible transcription factors (HIF). As such a direct link was established between methylation of CpG dinucleotides in HIF-binding sites and repellence of HIF-binding. Upon comparing HIF binding patterns between hypoxic wild-type murine embryonic stem cells and their DNA methyltransferase-lacking mutant counterparts, a surprising enrichment specific to the mutant cells was established of HIF binding sites in retrotransposons. This enrichment was moreover functional as correlating with retrotransposon expression, thus indicating repression of retrotransposon expression under hypoxic conditions, mediated by epigenetic silencing/methylation. Subsequently, a similar enrichment of HIF-binding sites in retrotransposons was established in human cancer cells. In further work, and with the help of de novo transcript assembly, a series of retrotransposons were identified for which the expression correlates with outcome of immunotherapy; these retrotransposons thus have diagnostic and theranostic potential. This series of retrotransposons is limited in number, and around 70% of the identified retrotransposons was not previously annotated.

Therefore, the invention relates in general to methods of tumor analysis or of tumor profiling, such methods comprising the step of detecting, determining or measuring in a sample obtained from a subject having the tumor, the expression level of at least one retrotransposon, or a change in the expression level of at least one retrotransposon compared to the expression level of the same retrotransposon in a control sample or compared to a standard value, wherein the at least one retrotransposon is selected from the (group of) retrotransposons (consisting of) HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5. Table 3 and Table 5 provide the information of the location of the retrotransposons on the indicated chromosome. In particular, the change in expression level is an increase in expression level compared to the expression level a control sample or compared to a standard value.

In another aspect, the invention relates to methods of determining or predicting, prior to start or early after start of immunotherapy or of an immunogenic therapy, the outcome of the immunotherapy or the immunogenic therapy, or of determining or predicting susceptibility to the immunotherapy or immunogenic therapy of a tumor in a subject, such methods comprising the step of detecting, determining or measuring the expression level of at least one retrotransposon, or of detecting, determining or measuring a change in the expression level of at least one retrotransposon, in a sample obtained from the subject, wherein the at least one retrotransposon is selected from the (group of) retrotransposons (consisting of) HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein an increased expression level of the at least one retrotransposon in the sample compared to the expression level of the same retrotransposon in a control sample or compared to a standard value, is indicative of a positive outcome of the immunotherapy or the immunogenic therapy, or is indicative of susceptibility of the tumor to the immunotherapy or immunogenic therapy.

Another aspect of the current invention relates to methods of determining or predicting the response to immunotherapy or immunogenic therapy of a tumor in a subject, comprising the step of detecting, determining or measuring the expression level of at least one retrotransposon, or of detecting, determining or measuring a change in the expression level of at least one retrotransposon, in a sample obtained from the subject, wherein the retrotransposon is selected from the (group of) retrotransposons (consisting of) HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein a decrease in the expression level of the at least one retrotransposon in the sample compared to the expression level of the same at least one retrotransposon in a sample obtained from the subject prior to immunotherapy or immunogenic therapy or in a sample obtained at or taken at an earlier time-point during immunotherapy or immunogenic therapy, is indicative of a positive response to the immunotherapy or immunogenic therapy.

Responses to therapy are explained in more detail in the section “Treatment/therapeutically effective amount” hereinafter.

A further aspect of the invention relates to methods of determining or predicting the presence of neo-epitopes in a tumor in a subject, comprising the step of c change in the expression level of at least one retrotransposon, in a sample obtained from the subject, wherein the retrotransposon is selected from the (group of) retrotransposons (consisting of) HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein an increased expression level of the at least one retrotransposon in the sample compared to the expression level of the same at least one retrotransposon in a control sample or compared to a standard value, is indicative of the presence of neo-epitopes in the tumor; and wherein a decrease in the expression level of the at least one retrotransposon in the sample compared to the expression level of the same at least one retrotransposon in a sample obtained from the subject prior to immunotherapy or immunogenic therapy or in a sample obtained at or taken at an earlier time-point during immunotherapy or immunogenic therapy, is indicative of a decreasing presence of neo-epitopes in the tumor.

Neo-epitopes are (parts of) peptides carrying tumor-specific mutations. As these appear in tumours, these are non-self epitopes (Brennick et al. 2017, Immunotherapy 9:361-371). Release of neo-epitopes from a tumor may in some circumstances be enhanced upon immunogenic death of tumor cells (such as by immunogenic therapy). Neo-epitopes or neo-antigens do not need to be of peptidic nature as innate immune responses can be raised to nucleic acids perceived as foreign.

In particular to all of the above methods, the retrotransposons are human retrotransposons, and the subject is a human subject or patient.

The contents of Tables 3 and 5 are explained hereafter in some more detail. As retrotransposon elements of a single family/group/class can occur on multiple genomic locations (e.g. MIRb is occurring on chromosomes 16 and 2 in Table 3), the chromosome of origin is included in the retrotransposon annotation (“chr n”, wherein “n” is the chromosome number, chromosome X, or chromosome Y). The L1PB4 retrotransposon motif is listed in Table 3 two times, both occurring on chromosome 9; therefore, an own further annotation “_1” or “_2” was added to distinguish both; L1PB4_2 is also listed in Table 5. Particularly relevant for defining the retrotransposons, such as for diagnostic or theranostic purposes, are the chromosome allocation (column “Chr” referring to chromosome number, as already included in the annotation) and the location of the retrotransposon on the allocated chromosome (start and end point; and forward (+) or reverse (−) strand where known). Retrieving the actual nucleic acid sequence from the indicated allocation on the indicated chromosome is known to the skilled person, and the actual nucleic acid sequence can be retrieved e.g. by using a genome browser (e.g. https://genome.ucsc.edu/or https://www.ncbi.nlm.nih.gov/genome/) and by relying on the reference human genome used to delineate the retrotransposons of the invention as listed in Tables 3 and 5 (see Examples 2.12 and 2.13 for these details). Tables 3 and 5 are defining the retrotransposons in terms of the retrotransposon family/group/class to which they belong (column “retrotransposon”). For example Kojima 2018 (Mobile DNA 9:2) is referred to for an overview of retrotransposon nomenclature. This reference allows a skilled person to categorize all of the retrotransposons listed in Tables 3 and 5 in terms of family, group or class. Notably, alternative annotations may sometimes be used, e.g. L1 PB3 instead of L1PB3, MamGyp instead of MamGypsy. Tables 3 and 5 further define the retrotransposons of the invention in terms of their Ensembl identificator if available (which can be queried e.g. via the Biomart function of Ensembl: http://www.ensembl.org/biomart/). Further description of the retrotransposons included in Tables 3 and 5 include their differential expression in responders to immune checkpoint therapy compared to non-responders to immune checkpoint therapy (column under the header “DE in”; see Examples 2.12 and 2.13). Some of the retrotransposons as listed in Table 3 are gender-specific and include HERVE_a-int (chrY), HERVK14C-int (chrY), HERV17-int (chrY), L1ME2 (chrY); Table 5 does not list retrotransposons located on chrY. These form a subset of retrotransposon biomarkers as described herein that are only relevant to subjects carrying the Y chromosome. The remainder (50) of the retrotransposon biomarkers as defined in Table 3 are gender-neutral/not gender-specific: HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11). About 70% of the retrotransposons listed in Table 3 where not annotated before. Table 5 lists 33 biomarkers of which 21 also are listed in Table 3-additional information is provided in terms of predictive accuracy and expression levels (expressed as RPKM). The 12 additional biomarkers of Table 5 relative to Table 3 are IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10). About 78% of the retrotransposons listed in Table 5 where not annotated before.

In contrast to DNA transposons which move as DNA, retrotransposons duplicate via RNA intermediates that are reverse transcribed. Retrotransposons comprise two subclasses: the long terminal repeat (LTR) and the non-LTR retrotransposons. The length of LTRs in LTR retrotransposons ranges from ˜100 bp to over 5 kb in size; the sequences are repeated directly at the 5′ and 3′ ends of LTR retrotransposons and retroviruses. Sub-classifications of LTR retrotransposons include Ty1-copia-like (Pseudoviridae), Ty3-gypsy-like (Metaviridae), and BEL-Pao-like retrotransposons. Several types of non-LTR retrotransposons are distinguished: autonomous long interspersed nuclear elements (LINEs, or L1), non-autonomous short interspersed nuclear elements (SINEs), and SVA elements. SINEs need at least the LINE machinery for retrotransposition (propagation via RNA intermediate). LINEs and SINEs make up approximately 17% and 11%, respectively, of the human genome. Most of the LINEs can no longer retrotranspose. The most common primate SINE is Alu, approximately 350 bp long, is comprising the Alul restriction enzyme site, and constitutes approximately 11% of the human genome. SVA elements comprise a SINE region, a variable number of tandem repeats (VNTR-region) and an Alu-like region. Retroviruses are usually classified separately but can share features with LTR retrotransposons. Compared to Ty1-copia and Ty3-gypsy retrotransposons, retroviruses have an Envelope protein (ENV). A retrovirus can transform into an LTR retrotransposon upon inactivation or deletion of domains that enable extracellular mobility. Infection of such retrovirus with subsequent insertion in a germ cell line genome may lead to vertical transmission. Such retrovirus then becomes an Endogenous Retrovirus (ERV); about 8% of the human genome and about 10% of the mouse genome consists of ERVs. More information on retrotransposons can be found in e.g. Cordeaux & Batzer 2009 (Nat Rev Genet 10:691-703), Criscione et al. 2014 (BMC Genomics 15:583), and Kojima 2018 (Mobile DNA 9:2).

In one embodiment, any of the above methods of (a) tumor analysis or tumor profiling, or (b) of determining or predicting, prior to or early after start of immunotherapy or of immunogenic therapy, the outcome of the immunotherapy or immunogenic therapy, or of determining or predicting susceptibility to the immunotherapy or immunogenic therapy of a tumor in a subject, (c) of determining or predicting response to immunotherapy or immunogenic therapy of a tumor in a subject, or (d) of determining or predicting the presence of neo-epitopes in a tumor in a subject, may entail/encompass/comprise detecting, determining or measuring the expression level, or of detecting, determining or measuring a change in the expression level of at least one (1) retrotransposon, of more than 1 retrotransposon, such as detecting, determining or measuring the expression level, or of detecting, determining or measuring a change in the expression level of 2 retrotransposons, of at least 2 retrotransposons, of 3 retrotransposons, of at least 3 retrotransposons, of 4 retrotransposons, of at least 4 retrotransposons, of 5 retrotransposons, of at least 5 retrotransposons, of 6 retrotransposons, of at least 6 retrotransposons, of 7 retrotransposons, of at least 7 retrotransposons, of 8 retrotransposons, of at least 8 retrotransposons, of 9 retrotransposons, of at least 9 retrotransposons, of 10 retrotransposons, of at least 10 retrotransposons, of 11 retrotransposons, of at least 11 retrotransposons, of 12 retrotransposons, of at least 12 retrotransposons, of 13 retrotransposons, of at least 13 retrotransposons, of 14 retrotransposons, of at least 14 retrotransposons, of 15 retrotransposons, of at least 15 retrotransposons, of 16 retrotransposons, of at least 16 retrotransposons, of 17 retrotransposons, of at least 17 retrotransposons, of 18 retrotransposons, of at least 18 retrotransposons, of 19 retrotransposons, of at least 19 retrotransposons, of 20 retrotransposons, of at least 20 retrotransposons, of 21 retrotransposons, of at least 21 retrotransposons, of 22 retrotransposons, of at least 22 retrotransposons, of 23 retrotransposons, of at least 23 retrotransposons, of 24 retrotransposons, of at least 24 retrotransposons, of 25 retrotransposons, of at least 25 retrotransposons, of 26 retrotransposons, of at least 26 retrotransposons, of 27 retrotransposons, of at least 27 retrotransposons, of 28 retrotransposons, of at least 28 retrotransposons, of 29 retrotransposons, of at least 29 retrotransposons, of 30 retrotransposons, of at least 30 retrotransposons, of 31 retrotransposons, of at least 31 retrotransposons, of 32 retrotransposons, of at least 32 retrotransposons, of 33 retrotransposons, of at least 33 retrotransposons, of 34 retrotransposons, of at least 34 retrotransposons, of 35 retrotransposons, of at least 35 retrotransposons, of 36 retrotransposons, of at least 36 retrotransposons, of 37 retrotransposons, of at least 37 retrotransposons, of 38 retrotransposons, of at least 38 retrotransposons, of 39 retrotransposons, of at least 39 retrotransposons, of 40 retrotransposons, of at least 40 retrotransposons, of 41 retrotransposons, of at least 41 retrotransposons, of 42 retrotransposons, of at least 42 retrotransposons, of 43 retrotransposons, of at least 43 retrotransposons, of 44 retrotransposons, of at least 44 retrotransposons, of 45 retrotransposons, of at least 45 retrotransposons, of 46 retrotransposons, of at least 46 retrotransposons, of 47 retrotransposons, of at least 47 retrotransposons, of 48 retrotransposons, of at least 48 retrotransposons, of 49 retrotransposons, of at least 49 retrotransposons, of 50 retrotransposons, of at least 50 retrotransposons, of 51 retrotransposons, of at least 51 retrotransposons, of 52 retrotransposons, of at least 52 retrotransposons, of 53 retrotransposons, of at least 53 retrotransposons, of 54 retrotransposons, of at least 54 retrotransposons, of 55 retrotransposons, of at least 55 retrotransposons, of 56 retrotransposons, of at least 56 retrotransposons, of 57 retrotransposons, of at least 57 retrotransposons, of 58 retrotransposons, of at least 58 retrotransposons, of 59 retrotransposons, of at least 59 retrotransposons, of 60 retrotransposons, of at least 60 retrotransposons, of 61 retrotransposons, of at least 61 retrotransposons, of 62 retrotransposons, wherein the first and any additional retrotransposon or retrotransposons (being different from the first selected retrotransposon) is or are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5. Optionally hereto, such methods can be extended to detecting, determining or measuring the expression levels, or of detecting, determining or measuring a change in expression levels of 1 retrotransposon, of at least 1 retrotransposon, of 2 retrotransposons, of at least 2 retrotransposons, of 3 retrotransposons, of at least 3 retrotransposons, or of 4 retrotransposons selected from the retrotransposons HERVE_a-int (chrY), HERVK14C-int (chrY), HERV17-int (chrY), and L1ME2 (chrY), all as defined in Table 3 or wherein all retrotransposons are defined in Table 3.

Purely exemplary subsets or signatures of retrotransposons include those as compiled in the Examples 2.12 and 2.13 herein. The selection of these subsets or signatures was entirely driven by the subset of patient samples analyzed and is actually underlying the identification of the retrotransposons the expression of which is the central feature of the current invention. As explained in Examples 2.12 and 2.13, each of these subsets or signatures has diagnostic or theranostic power relative to the pool of all patients (wherein all patient subsets or cohorts are combined). This underscores the fact that multiple different signatures not comprising all of the retrotransposons
as defined in Table 3 (i.e. all of HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), eventually combined with the retrotransposons selected from the retrotransposons HERVE_a-int (chrY), HERVK14C-int (chrY), HERV17-int (chrY), and L1ME2 (chrY)), and/or
as defined in Table 5 (i.e. all of MSTD/AluSq2 (chr5), MLT1E2/MLT2B3 (chr5), L2b/FLAM_A (chr11), IncRNA1 (CU104787.1; chr22), L1MD1/L1M1 (chr3), L1PREC2 (chr3), MIRb/AluSz (chr16), MIRb (chrX), L1MC2 (chr4), MLT2C1 (chr5), L1ME4a/L2a (chr20), LTR12C (chrX), HERV9-int/AluY (chr12), L1PA17/MLT2E (chr16), AmnSINE1 (chrX), IncRNA2 (chrX), MLT1C (chr5), THE1C (chr4), L1MA6/MER4B (chr10), LTR1A2 (chr18), IncRNA3 (chr13), MSTA/MSTA-int (chr13), MER61A-int/MER61A (chr13), THE1b/AluYe5 (chr18), L1PB4_2 (chr9), MLT1G3 (chr13), L1MC3 (chr4), THE1A/L1PA16 (chr7), L1MC5 (chr10), AluSx1 (chr10), L1M5/AluSc (chr7), MIRb (chr18), and LTR12C (chr5)),

can be envisaged, and that such signatures containing a plurality of retrotransposons are certainly not restricted to those specifically outlined herein: the Sig30, Sig19, and Sig9 signatures, or, when omitting the retrotransposons located on the Y-chromosome, the Sig29, Sig17, and Sig8 signatures; or the Sig33, Sig24, Sig9b, and Sig4 signatures.

The Sig30 retrotransposon expression signature can be derived from Table 3 as the subset being differentially expressed in the Leuven and Hugo patient cohorts (see Example 2.12): HERV9-int/AluY (chr12), MSTA/MSTA-int (chr13), MER61-int/MER61A (chr13), L1PB3 (chr13), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), L1ME4a/L2a (chr20), LTR67B (chr20), L1MCa (chr21), L4_A_Mam (chr22), MLT1A1 (chr22), ERVL-B4-int (chr3), L1PREC2 (chr3), L1MD1/L1M1 (chr3), THE1D (chr4), MLT2C1 (chr5), L1PREC2 (chr5), L1MB8 (chr5), MLT1E2/MLT2B3 (chr5), L1MB4 (chr5), MSTD/AluSq2 (chr5), L1MA6/MER4B (chr10), L2 (chrX), L1ME2 (chrY), and L2b/FLAM_A (chr11). The Sig29 retrotransposon expression signature is lacking the retrotransposon L1ME2 (chrY) compared to the Sig30 retrotransposon expression signature.

The Sig19 retrotransposon expression signature can be derived from Table 3 as the subset being differentially expressed in the Leuven and Riaz patient cohorts (see Example 2.12): HERV9-int/AluY (chr12), L1M4 (chr13), MLT1G3 (chr13), AluSx3 (chr14), L1MC4a (chr14), MIRb (chr2), PRIMA41-int (chr22), THE1D (chr4), THE1B (chr4), L1MC3 (chr4), L1M4b (chr1), MLT1J1-int (chr6), L1M5/AluSc (chr7), THE1A/L1PA16 (chr7), L1PB4_1 (chr9), L1PB4_2 (chr9), HERVE_a-int (chrY), HERVK14C-int (chrY), and L1MC5 (chr10). The Sig17 retrotransposon expression signature is lacking the retrotransposons HERVE_a-int (chrY) and HERVK14C-int (chrY) compared to the Sig19 retrotransposon expression signature.

The Sig9 retrotransposon expression signature can be derived from Table 3 as the subset being differentially expressed in the Hugo and Riaz patient cohorts (see Example 2.12): HERV9-int/AluY (chr12), MER57E1 (chr13), L1ME3Cz (chr14), LTR16C (chr14), THE1C (chr2), L2a (chr3), THE1D (chr4), L1MB2 (chr5), and HERV17-int (chrY). The Sig8 retrotransposon expression signature is lacking the retrotransposon HERV17-int (chrY) compared to the Sig9 retrotransposon expression signature.

The Sig33 retrotransposon expression signature can be derived from Table 5 and consists of MSTD/AluSq2 (chr5), MLT1E2/MLT2B3 (chr5), L2b/FLAM_A (chr11), IncRNA1 (CU104787.1; chr22), L1MD1/L1M1 (chr3), L1PREC2 (chr3), MIRb/AluSz (chr16), MIRb (chrX), L1MC2 (chr4), MLT2C1 (chr5), L1ME4a/L2a (chr20), LTR12C (chrX), HERV9-int/AluY (chr12), L1PA17/MLT2E (chr16), AmnSINE1 (chrX), IncRNA2 (chrX), MLT1C (chr5), THE1C (chr4), L1MA6/MER4B (chr10), LTR1A2 (chr18), IncRNA3 (chr13), MSTA/MSTA-int (chr13), MER61A-int/MER61A (chr13), THE1b/AluYe5 (chr18), L1PB4_2 (chr9), MLT1G3 (chr13), L1MC3 (chr4), THE1A/L1PA16 (chr7), L1MC5 (chr10), AluSx1 (chr10), L1M5/AluSc (chr7), MIRb (chr18), and LTR12C (chr5).

The Sig24 retrotransposon expression signature can be derived from Table 5 as the subset being differentially expressed in the Leuven and Hugo patient cohorts (see Example 2.13): MSTD/AluSq2 (chr5), MLT1E2/MLT2B3 (chr5), L2b/FLAM_A (chr11), IncRNA1 (CU104787.1; chr22), L1MD1/L1M1 (chr3), L1PREC2 (chr3), MIRb/AluSz (chr16), MIRb (chrX), L1MC2 (chr4), MLT2C1 (chr5), L1ME4a/L2a (chr20), LTR12C (chrX), HERV9-int/AluY (chr12), L1PA17/MLT2E (chr16), AmnSINE1 (chrX), IncRNA2 (chrX), MLT1C (chr5), THE1C (chr4), L1MA6/MER4B (chr10), LTR1A2 (chr18), IncRNA3 (chr13), MSTA/MSTA-int (chr13), MER61A-int/MER61A (chr13), and THE1b/AluYe5 (chr18).

The Sig9b retrotransposon expression signature can be derived from Table 5 as the subset being differentially expressed in the Leuven and Riaz patient cohorts (see Example 2.13): MIRb (chrX), HERV9-int/AluY (chr 12), L1PB4_2 (chr9), MLT1G3 (chr13), L1MC3 (chr4), THE1A/L1PA16 (chr7), L1MC5 (chr10), AluSx1 (chr10), and L1M5/AluSc (chr7)

The Sig4 retrotransposon expression signature can be derived from Table 5 as the subset being differentially expressed in the Hugo and Riaz patient cohorts (see Example 2.13): MIRb (chr18), MIRb (chrX), HERV9-int/AluY (chr12), and LTR12C (chr5).

In a further embodiment, any of the above methods of (a) tumor analysis or tumor profiling, or (b) of determining or predicting, prior to or early after start of immunotherapy or of immunogenic therapy, the outcome of the immunotherapy or immunogenic therapy, or of determining or predicting susceptibility to the immunotherapy or immunogenic therapy of a tumor in a subject, (c) of determining or predicting response to immunotherapy or immunogenic therapy of a tumor in a subject, or (d) of determining or predicting the presence of neo-epitopes in a tumor in a subject, may entail detecting, determining or measuring the expression level, or of detecting, determining or measuring a change in the expression level of at least one retrotransposon (or of a first selected retrotransposon) wherein said at least one retrotransposon (or said first selected retrotransposon) is HERV9-int/AluY (chr12), THE1D (chr4), or MIRb (chrX), as defined in Table 3 or Table 5.

Furthermore, corroborating the lack of restriction imposed by the exemplary subsets or signatures of retrotransposons (see above), as explained in Examples 2.12 and 2.13, and as apparent from FIGS. 11, 12, 17, 18, 22, 26 and 28, the expression level of a selected set of retrotransposons can be analysed, and only a change in the expression level of at least one of selected retrotransposons can be sufficient as being indicative of a positive response of a tumor to immunotherapy or immunogenic therapy [by means of detecting increased expression, relative to a proper control, of a retrotransposon prior to or shortly after start of immunotherapy or of immunogenic therapy; or by means of detecting decreased expressed of a retrotransposon after start of immunotherapy or of immunogenic therapy relative to the expression level of the retrotransposon before start of, or relative to earlier during immunotherapy or of immunogenic therapy], or of the presence of neo-epitopes in a tumor. For instance, when analysing expression of the retrotransposons in either one of the Sig54, Sig33, Sig30, Sig29 or Sig19 signatures, increased expression of at least 3 retrotransposons of these signatures prior to or shortly after onset of immunotherapy or immunogenic therapy is sufficient for being indicative of a positive response of a tumor to immunotherapy or immunogenic therapy (for the Sig30 signature, increased expression of 2, 3 or 4 retrotransposons is sufficient, see FIG. 11). The same holds true for the Sig17, Sig9, Sig9b, Sig8 and Sig4 signatures in which increased expression of at least 1 retrotransposon of these signatures prior to or shortly after onset of immunotherapy or immunogenic therapy is sufficient for being indicative of a positive response of a tumor to immunotherapy or immunogenic therapy. In the same manner, for the Sig24 signature, increased expression of at least 2 retrotransposons is sufficient. This illustrates that a change in expression of 1 retrotransposon out of a set of 4 to 18 retrotransposons is sufficient. Table 5 provides further information on the predictive accuracy of the individual biomarkers. Logically, the size of a biomarker set assembled with markers having the highest predictive accuracy is plausibly going to be smaller than the size of a biomarker set comprising markers with lower predictive accuracy. Notwithstanding this, the Sig4 biomarker set comprises 1 marker with 63% predictive accuracy, 2 markers with 61% predictive accuracy, and 1 marker with 60% predictive accuracy, and yet, detection of increased expression of 1 of the biomarkers of the Sig4 signature is sufficient.

Based hereon, in a further embodiment, any of the above methods of (a) tumor analysis or tumor profiling, or (b) of determining or predicting, prior to or early after start of immunotherapy or of immunogenic therapy, the outcome of the immunotherapy or immunogenic therapy, or of determining or predicting susceptibility to the immunotherapy or immunogenic therapy of a tumor in a subject, (c) of determining or predicting response to immunotherapy or immunogenic therapy of a tumor in a subject, or (d) of determining or predicting the presence of neo-epitopes in a tumor in a subject, may entail/encompass/comprise detecting/determining/assessing/assaying/measuring the expression level of more than 1 retrotransposon, such as detecting/determining/assessing/assaying/measuring the expression level of 4 retrotransposons, of at least 4 retrotransposons, of 5 retrotransposons, of at least 5 retrotransposons, of 6 retrotransposons, of at least 6 retrotransposons, of 7 retrotransposons, of at least 7 retrotransposons, of 8 retrotransposons, of at least 8 retrotransposons, of 9 retrotransposons, of at least 9 retrotransposons, of 10 retrotransposons, of at least 10 retrotransposons, of 11 retrotransposons, of at least 11 retrotransposons, of 12 retrotransposons, of at least 12 retrotransposons, of 13 retrotransposons, of at least 13 retrotransposons, of 14 retrotransposons, of at least 14 retrotransposons, of 15 retrotransposons, of at least 15 retrotransposons, of 16 retrotransposons, of at least 16 retrotransposons, of 17 retrotransposons, of at least 17 retrotransposons, of 18 retrotransposons, of at least 18 retrotransposons, of 19 retrotransposons, of at least 19 retrotransposons, of 20 retrotransposons, of at least 20 retrotransposons, of 21 retrotransposons, of at least 21 retrotransposons, of 22 retrotransposons, of at least 22 retrotransposons, of 23 retrotransposons, of at least 23 retrotransposons, of 24 retrotransposons, of at least 24 retrotransposons, of 25 retrotransposons, of at least 25 retrotransposons, of 26 retrotransposons, of at least 26 retrotransposons, of 27 retrotransposons, of at least 27 retrotransposons, of 28 retrotransposons, of at least 28 retrotransposons, of 29 retrotransposons, of at least 29 retrotransposons, of 30 retrotransposons, of at least 30 retrotransposons, of 31 retrotransposons, of at least 31 retrotransposons, of 32 retrotransposons, of at least 32 retrotransposons, of 33 retrotransposons, of at least 33 retrotransposons, of 34 retrotransposons, of at least 34 retrotransposons, of 35 retrotransposons, of at least 35 retrotransposons, of 36 retrotransposons, of at least 36 retrotransposons, of 37 retrotransposons, of at least 37 retrotransposons, of 38 retrotransposons, of at least 38 retrotransposons, of 39 retrotransposons, of at least 39 retrotransposons, of 40 retrotransposons, of at least 40 retrotransposons, of 41 retrotransposons, of at least 41 retrotransposons, of 42 retrotransposons, of at least 42 retrotransposons, of 43 retrotransposons, of at least 43 retrotransposons, of 44 retrotransposons, of at least 44 retrotransposons, of 45 retrotransposons, of at least 45 retrotransposons, of 46 retrotransposons, of at least 46 retrotransposons, of 47 retrotransposons, of at least 47 retrotransposons, of 48 retrotransposons, of at least 48 retrotransposons, of 49 retrotransposons, of at least 49 retrotransposons, of 50 retrotransposons, of at least 50 retrotransposons, of 51 retrotransposons, of at least 51 retrotransposons, of 52 retrotransposons, of at least 52 retrotransposons, of 53 retrotransposons, of at least 53 retrotransposons, of 54 retrotransposons, of at least 54 retrotransposons, of 55 retrotransposons, of at least 55 retrotransposons, of 56 retrotransposons, of at least 56 retrotransposons, of 57 retrotransposons, of at least 57 retrotransposons, of 58 retrotransposons, of at least 58 retrotransposons, of 59 retrotransposons, of at least 59 retrotransposons, of 60 retrotransposons, of at least 60 retrotransposons, of 61 retrotransposons, of at least 61 retrotransposons, of 62 retrotransposons; and detecting/determining/assessing/assaying/measuring a change in expression of 1 retrotransposon, of at least 1 retrotransposon, of 2 retrotransposons, of at least 2 retrotransposons, of 3 retrotransposons, of at least 3 retrotransposons, of 4 retrotransposons, or of at least 4 retrotransposons of the set of retrotransposons for which the expression level has been determined. In particular, in these methods, a change in expression is detected/determined/assessed/assayed/measured for at least 1 retrotransposon in a set of 4 retrotransposons, for at least 1 retrotransposon in a set of 5 retrotransposons, for at least 1 retrotransposon in a set of 6 retrotransposons, for at least 1 retrotransposon in a set of 7 retrotransposons, for at least 1 retrotransposon in a set of 8 retrotransposons, for at least 1 retrotransposon in a set of 9 retrotransposons, for at least 1 retrotransposon in a set of 10 retrotransposons, for at least 1 retrotransposon in a set of 11 retrotransposons, for at least 1 retrotransposon in a set of 12 retrotransposons, for at least 1 retrotransposon in a set of 13 retrotransposons, for at least 1 retrotransposon in a set of 14 retrotransposons, for at least 1 retrotransposon in a set of 15 retrotransposons, for at least 1 retrotransposon in a set of 16 retrotransposons, for at least 1 retrotransposon in a set of 17 retrotransposons, or for at least 1 retrotransposon in a set of 18 retrotransposons. Herein the (set of) retrotransposons for which the expression level is detected/determined/assessed/assayed/measured are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5. Optionally these retrotransposons can further be selected from the retrotransposons HERVEa-int (chrY), HERVK14C-int (chrY), HERV17-int (chrY), and L1ME2 (chrY), all as defined in Table 3 or wherein all retrotransposons are defined in Table 3. Optionally, at least one of the selected retrotransposons is selected from HERV9-int/AluY (chr12), THE1D (chr4), or MIRb (chrX), as defined in Table 3 or Table 5. The relative change in expression level is outlined hereinabove.

The control sample referred to in the above methods is a sample of a healthy subject or is a mixture of samples of one or more healthy subjects. Instead of a control sample, a standard value of expression of an individual retrotransposon can be used for purposes of detecting changes in retrotransposon expression compared to such standard value. A standard value of retrotransposon expression may for instance be derived or averaged from cell-, tissue-, or organ samples of a plurality of subjects not having a tumor but wherein the cell-, tissue-, or organ samples used for determining the standard value are of the same type as the cell-, tissue-, or organ samples taken from a tumor which is to be analyzed for detecting changes in retrotransposon expression relative to the standard value of retrotransposon expression. Alternatively, the control sample is a tumor sample, or one of a series of tumor samples, of the same subject having the tumor but taken at an earlier time-point compared to the tumor sample to be newly analyzed (detection of changes in retrotransposon expression of a later tumor sample compared to an earlier tumor sample of the same subject). Such earlier time or time-point may be before or early after start of any therapy (such as immune checkpoint therapy, immunotherapy or immunogenic therapy; “early after” herein is meant a period of time during which the therapy has not yet significantly affected the disease targeted by the therapy), or at any earlier time or time-point after start of any therapy (such as immune checkpoint therapy, immunotherapy or immunogenic therapy) but preceding collection of the sample to be newly analyzed. In some instances, the expression of a retrotransposon of the invention in a control sample, or the standard value for expression of a retrotransposon of the invention, can be “zero”, or below the detection limit.

Increase or decrease of expression of a retrotransposon of the invention thus is relative to a control sample or standard value as described above. In case of a “zero” value for the control sample of standard value, a change of expression of an individual retrotransposon from “zero” to a value equal to or higher than the detection limit is considered as increased expression of that individual retrotransposon. In case of a value for the control sample of standard value equal to or higher than the detection limit, a change of expression of an individual retrotransposon is considered as increased expression of that individual retrotransposon upon increase in the number of analyte strands (see further) of 5% or more, of 10% or more, of 15% or more, of 20% or more, or 25% or more, of 30% or more, of 35% or more, of 40% or more, of 45% or more, of 50% or more, or 55% or more, of 60% or more, of 65% or more, of 70% or more, of 75% or more, of 80% or more, of 85% or more, of 90% or more, of 95% or more, of 100% or more, of up to 10%, up to 20%, of up to 30%, of up to 40%, of up to 50%, of up to 60%, of up to 70%, of up to 80%, of up to 90%, or of up to 100%. In case of a 100% increase in analyte strand number of an individual retrotransposon, the expression of that individual transposon has doubled, or increased 2-fold. The increase in analyte strand number of an individual retrotransposon can further be a 1.1-fold increase, a 1.2-fold increase, a 1.3-fold increase, a 1.4-fold increase, a 1.5-fold increase, a 1.6-fold increase, a 1.7-fold increase, a 1.8-fold increase, a 1.9-fold increase, a 2-fold increase, a 2.1-fold increase, a 2.2-fold increase, a 2.3-fold increase, a 2.4-fold increase, a 2.5-fold increase, a 2.6-fold increase, a 2.7-fold increase, a 2.8-fold increase, a 2.9-fold increase, a 3-fold increase, a higher than 3-fold increase, a 3.5-fold increase, a 4-fold increase, a higher than 4-fold increase, an increase of between 3-fold and 4-fold, a 4.5-fold increase, a 5-fold increase, a higher than 5-fold increase, an increase of between 2-fold and 5-fold, an increase of between 3-fold and 5-fold, an increase of between 3-fold and 5-fold, a 6-fold increase, a higher than 6-fold increase, an increase of between 2-fold and 6-fold, an increase of between 3-fold and 6-fold, an increase of between 4-fold and 6-fold, a 7-fold increase, a higher than 7-fold increase, an 8-fold increase, a higher than 8-fold increase, a 9-fold increase, a higher than 9-fold increase, a 10-fold increase, an up to 10-fold increase, a higher than 10-fold increase, an increase of between 2-fold and 10-fold, an increase of between 3-fold and 10-fold, an increase of between 4-fold and 10-fold, an increase of between 5-fold an 10-fold, an increase of between 6-fold and 10-fold, an increase of between 7-fold and 10-fold, an increase of between 8-fold and 10-fold, a substantially higher than 10-fold increase, an increase of between 10-fold and 15-fold, an up to 15-fold increase, an increase of between 10-fold and 20-fold, un up to 20-fold increase, a substantially higher than 20-fold increase such as an up to 25-fold increase, an up to 30-fold increase, an up to 40-fold increase, or an up to 50-fold increase.

Considered as decreased expression of an individual retrotransposon is a decrease in the number of analyte strands (see further) of 5% or more, of 10% or more, of 15% or more, of 20% or more, or 25% or more, of 30% or more, of 35% or more, of 40% or more, of 45% or more, of 50% or more, or 55% or more, of 60% or more, of 65% or more, of 70% or more, of 75% or more, of 80% or more, of 85% or more, of 90% or more, of 95% or more, of 100%, of up to 10%, up to 20%, of up to 30%, of up to 40%, of up to 50%, of up to 60%, of up to 70%, of up to 80%, of up to 90%, or of up to 100%. Further considered as decreased expression of an individual retrotransposon is a decrease in the number of analyte strands from equal to or higher than the detection limit to below the detection limit.

Any of the above described methods of (a) tumor analysis or tumor profiling, or (b) of determining or predicting, prior to or early after start of immunotherapy or immunogenic therapy, the outcome of the immunotherapy or immunogenic therapy, or of determining or predicting susceptibility to the immunotherapy or immunogenic therapy of a tumor in a subject, (c) of determining or predicting response to immunotherapy or immunogenic therapy of a tumor in a subject, or (d) of determining or predicting the presence of neo-epitopes in a tumor in a subject, may be supplemented with one or more steps of determining/assessing/assaying/detecting/measuring the status of further diagnostic markers or biomarkers. Such has in part been performed in Examples 2.12 and 2.13 herein, and included other diagnostic markers assessed were nucleotide substitution number (whole exome sequencing, WES), number of indels (insertions or deletions; WES), immune cytolytic activity, T cell-inflamed gene expression signature, IFN-γ related gene expression signature, type I and type II interferon-related gene expression, the immunopredictive score, and expression of immune checkpoint genes.

As indicated by Cristescu et al. 2018 (Science 362:197) for instance, expression of ligand 1 of the immune checkpoint gene PD-1 (PD-L1) is a clinically validated biomarker for response to the PD-1-inhibitor pembrolizumab, just as is high microsatellite instability (the latter regardless of tumor type). The same authors elaborated on the applicability of tumor mutational burden (TMB) and the T cell-inflamed gene expression profile (GEP) as biomarkers for predicting response to immune checkpoint inhibition therapy, and concluded that both sets independently predict response, but nevertheless provide complementary information enabling subgrouping of tumours which may further guide precision cancer immunotherapy. This study highlights the importance of tumor analysis or of tumor profiling based on different sets of marker types (such as related to the tumor cell status, status of immune compartment of the tumor, and status of the tumoral microenvironment), even if the individual sets themselves already have some independent predictive value. Thus, another biomarker that can be combined with the herein described retrotransposon expression markers is immune cytolytic activity, such as developed by Rooney et al. 2015 (Cell 160:48-61) and which relies on determination of transcript levels of two cytolytic effectors, granzyme A (GZMA) and perforin (PRF1). Other possible markers complementary or supplementary to the herein described retrotransposon expression markers are interferon-related gene expression signatures. Such genes are e.g. genes known to be induced by type I interferons, type II interferons (see, e.g., Hall et al. 2012, Proc Natl Acad Sci USA 109:17609-17614; and see Example 12) or by interferon gamma (IFN-γ). A limited IFN-γ signature contains the genes IFNG, STAT1, CCR5, CXCL9, CXCL10, CXCL11, IDO1, PRF1, GZMA, and MHCII HLA-DRA (note that the cytolytic markers PRF1 and GZMA are included herein). An extended IFN-γ signature contains further cytolytic markers, chemokine and chemokine receptors, T cell markers, markers of NK cell activity, antigen presentation genes and immunomodulatory factors: granzyme B (GZMB), granzyme K (GZBK), CXCR6, CCL5, CD3D, CD3E, CD2, CXCL13, CXCL10, IL2RG, NKG7, HLA-E, CIITA, LAG3, IDO1, SLAMF6, TAGAP, STAT1 (Ayers et al. 2017, J Clin Invest 127:2930-2940).

High microsatellite instability (MSI) has been recognized by the FDA as a relevant biomarker for predicting response to anti-PD-1 therapy (see above). Historical markers of MSI include the markers of the revised Bethesda panel (Boland et al., 1998; Dietmaier et al., 1997), including the markers BAT25, BAT26, D5S346, D175250, D2S123, BAT40, D175787, D18S58, D18569, and TGFβ-RII. Based on whole genome sequencing analysis, many more MSI marker were identified in WO 2013/153130, including indel mutations in homopolymer sequences occurring in 5′UTR, 3′UTR, and exon regions of several genes (see Tables 1 and 2 of WO 2013/153130). Determination of MSI status can be combined with the herein described detection of changes in expression of the retrotransposon markers of the invention. MSI can be considered in part to contribute to the overall tumor mutation load or burden (TMB). Tumor mutational burden can also be determined by sequencing genes known to be subject to mutation in tumours. Table 5 of US 2019/0018926 provides an extensive list of “mutational burden genes”. In general, US 2019/0018926 relates to methodologies for generating an immune-oncology profile of a given tumor sample. Detecting the tumor mutation burden and/or generating an immune-oncology profile of tumor (such as by the methods of US 2019/0018926) can be combined with the herein described detection of changes in expression of the retrotransposon markers of the invention.

As indicated herein, hypoxia and retrotransposon expression are correlated. As such, increased expression of hypoxia marker genes is a source of further possible markers complementary or supplementary to the herein described retrotransposon expression markers. Such hypoxia marker genes can be one or more of the genes BNIP3, EGLN3, CA9, or ALDOA as used herein; or can be one or more of the genes described by Sorensen et al. 2015 (Radiother Oncol 116:346-351): ADM (adrenomedullin), ALDOA (Aldolase, Fructose-Bisphosphate A), ANKRD37 (Ankyrin Repeat Domain 37), BNIP3 (BCL2 Interacting Protein 3), BNIP3L (BCL2 Interacting Protein 3-Like), EGLN3 (Egl-9 Family Hypoxia Inducible Factor 3), FAM162A (Family With Sequence Similarity 162 Member A), KCTD11 (Potassium Channel Tetramerization Domain Containing 11), LOX (Lysyl Oxidase), NDRG1 (N-Myc Downstream Regulated 1), P4HA1 (Prolyl 4-Hydroxylase Subunit Alpha 1), P4HA2 (Prolyl 4-Hydroxylase Subunit Alpha 2), PDK1 (Pyruvate Dehydrogenase Kinase 1), PFKB3 (6-Phosphofructo-2-Kinase/Fructose-2,6-Biphosphatase 3), SLC2A1 (Solute Carrier Family 2 Member 1, also known as GLUT1). These authors also mention OPN (osteopontin) and LDH (lactate dehydrogenase) as hypoxia markers. An alternative way to determine the hypoxia status of a tumor, and thus alternative markers therefore, is by determining the hypermethylation status of one or more promoters of tumor suppressor genes (TSGs) HIC1, KDM6A, NF2, KDMSC, IGFBP2, ARNT2, PTEN, MGMT, ATM, MLH1, BRCA1, SEMA3B, TIMP3, THBD, and CLDN3. Increased hypermethylation in TSG promoters is indicative of a hypoxic tumor (see WO 2016/142295A1). Other markers providing information about a tumor or its environment that can complement or supplement the herein described retrotransposon expression marker include the detection of the expression of immunomodulatory genes. Examples of immune modulatory molecules include, but are not limited to, one or more of 2B4 (CD244), A2aR, B7H3 (CD276), B7H4 (VTCN1), B7H6, B7RP1, BTLA (CD272), butyrophilins, CD103, CD122, CD137 (4-1BB), CD137L, CD160, CD2, CD200R, CD226, CD26, CD27, CD28, CD30, CD39, CD40, CD48, CD70, CD73, CD80 (B7.1), CD86 (B7.2), CEACAM1, CGEN-15049, CTLA-4, DR3, GALS, GITR, GITRL, HVEM, ICOS, ICOSL (B7H2), IDOL, IDO2, ILT-2 (LILRB1), ILT-4 (LILRB2), KIR, KLRG1, LAG3, LAIR1 (CD305), LIGHT (TNFSF14), MARCO, NKG2A, NKG2D, OX-40, OX-40L, PD-1, PDL-1 (B7-H1, CD 274), PDL-2 (B7-DC, CD 273), PS, SIRPalpha, CD47, SLAM, TGFR, TIGIT, TIM1, TIM3 (HAVCR2), TIM4, or VISTA. In one specific setup, the expression of immune checkpoint genes is used to build the immune-predictive score (IMPRES; Auslander et al. 2018, Nature Med 24:1545-1549) relying on immune checkpoint inhibitors (ADORA2A, BTLA, VISTA, CD200, CD200R1, PDL-1, CD276, CD80, CD86, CEACAM1, CTLA4, GAL3, TIM-3, IDO1, KIR3DL1, LAG3, LAIR1, PD-1, PD-1LG2, PVR, PVRL2, TIGIT, VTCN1) and immune checkpoint activators (CD266, CD27, CD28, CD40, CD40L, CD70LG, DR3, HAVCR1, ICOS, ICOSL, IL2RB, NAIL, SLAM, TIM2, HVEM, TNFRSF18, TNFRSF4, TNFRSF9, TNFSF14, TNFSF18, OX40L, CD137L) which are paired (see Auslander et al. 2018 for details). Again, detection of changes in expression of the retrotransposons of the current invention can be combined with determining such immune-predictive score. Detection of expression of or of changes in expression of the retrotransposons of the current invention can further be combined with detecting expression of one or more immune checkpoint inhibitors and/or with detecting expression of one or more immune checkpoint activators. Immune checkpoint inhibitor genes include ADORA2A, BTLA, VISTA, CD200, CD200R1, PDL-1, CD276, CD80, CD86, CEACAM1, CTLA4, GAL3, TIM-3, IDO1, KIR3DL1, LAG3, LAIR1, PD-1, PD-1LG2, PVR, PVRL2, TIGIT, and VTCN1. Immune checkpoint activator genes include CD266, CD27, CD28, CD40, CD40L, CD70LG, DR3, HAVCR1, ICOS, ICOSL, IL2RB, NAIL, SLAM, TIM2, HVEM, TNFRSF18, TNFRSF4, TNFRSF9, TNFSF14, TNFSF18, OX40L, and CD137L.

Further markers providing information about a tumor or its environment that can complement or supplement the herein described retrotransposon expression marker include detecting the status of an innate anti-PD-1 resistance gene expression signature (IPRES) such as described by Hugo et al. 2016 (Cell 165:35-44). The IPRES scoring relying on enrichment of an innate anti-PD-1 resistance gene expression signature including genes involved in mesenchymal transition, angiogenesis, hypoxia, and wound healing (see Hugo et al. 2016, Cell 165:35-44 for details).

Detecting the tumor immune cell composition in conjunction with detection of changes in expression of the retrotransposon expression markers as described herein may also provide additional information about the tumor status. Examples of immune cells to be detected by methods described herein include, but are not limited to, CD4+ memory T-cells, CD4+ naive T-cells, CD4+ T-cells, central memory T (Tcm) cells, effector memory T (Tem) cells, CD4+ Tcm, CD4+ Tem, CD8+ T-cells, CD8+ naive T-cells, CD8+)Tcm, CD8+ Tem, regulatory T cells (Tregs), T helper (Th) 1 cells, Th2 cells, gamma delta T (Tgd) cells, natural killer (NK) cells, natural killer T (NKT) cells, B-cells, naive B-cells, memory B-cells, class-switched memory B-cells, pro B-cells, and plasma cells. In some instances, the sequencing data is used to determine expression of non-immune cells including, but not limited to, stromal cells, stem cells, or tumor cells. As indicator of the presence and/or amount of CD4+ T-cells, the expression of one or more of the following genes can be determined (US 2019/0018926, Table 1A): ALS2CL, ANKRD55, ZNF483, TRAV13-1, ST6GALNAC1, SEMA3A, TRBV5-4, DNAH8, IL2RA, TRBV11-2, TRAV8-2, KRT72, EPPK1, FAM153B, TRAV12-2, TRAV8-6, TRBV6-5, TRAV10, IGKV5-2, IGLV6-57, TRAV12-1, CTLA4, TSHZ2, FOXP3, IGHV4-28, TRAV2, SORCS3, TRAYS, MDS2, NTN4, IGLV10-54, DACT1, TRBV5-5, THEMS, HPCAL4, and/or CD4. As indicator of the presence and/or amount of CD8+ T-cells, the expression of one or more of the following genes can be determined (US 2019/0018926, Table 1B): FLT4, TRBV4-2, TRBV6-4, SPRY2, S100B, TNIP3, CD248, ROBO1, CD8B, TRBV2, CYP4F22, PZP, LAG3, KLRC4-KLRK1, CRTAM, SHANK1, ANAPC1P1, NRCAM, JAKMIP1, KLRC2, KLRC3, CD8A, TRAV4, FBLN2. As indicator of the presence and/or amount of monocytes, the expression of one or more of the following genes can be determined (US 2019/0018926, Table 1C): DES, HLX, FPR3, FCGR1B, LOXHD1, EPHB2, LPL, LIPN, AQP9, MILR1, RETN, GPNMB, CYP2S1, PDK4, LILRA6, SEPT10, PLA2G4A, FOLR2, FOLR3, C1QB, SLC6A12, SLC22A16, DOCK1, NRG1, RXFP2, RIN2, ARHGEF10L, LPAR1, CES1, FPR2. As indicator of the presence and/or amount of natural killer (NK) cells, the expression of one or more of the following genes can be determined (US 2019/0018926, Table 1D): IGFBP7, LDB2, GUCY1A3, KLRF1, DTHD1, AKR1C3, FASLG, KLRC1, XCL1, DAB2, FAT4, CD160, BNC2, CXCR1, SIGLEC17P, SH2D1B, DGKK, ZMAT4, LGALS9B, NMUR1, LGALS9C, MLC1, LIM2, NCR1, CCNJL, PCDH1. As indicator of the presence and/or amount of B-cells, the expression of one or more of the following genes can be determined (US 2019/0018926, Table 1E): UGT8, IGKV1OR2-108, IGHE, SCN3A, IGLV2-8, IGKV1D-16, MYO5B, ENAM, RP11-148021.2, IGLC7, IGHV1-2, IGKJ5, SOX5, TNFRSF13B, IGKV2D-29, IGKV1-17, IGLV2-18, IGHV2-70, CHL1, IGKV3D-20, IGLV8-61, IGKV6-21.

Based on the above, any of the hereinabove described methods of (a) tumor analysis or tumor profiling, or (b) of determining or predicting, prior to or early after start of immunotherapy or of immunogenic therapy, the outcome of the immunotherapy or immunogenic therapy or of determining or predicting susceptibility to the immunotherapy or immunogenic therapy of a tumor in a subject, (c) of determining or predicting response to immunotherapy or immunogenic therapy of a tumor in a subject, or (d) of determining or predicting the presence of neo-epitopes in a tumor in a subject, may be supplemented with one or more steps of determining/assessing/assaying/detecting/measuring the status of one or more further diagnostic markers or biomarkers. Such further diagnostic markers include immune checkpoint gene expression, markers of tumor mutational burden (such as substitutions, indels, microsatellite instability (MSI), providing substitution markers, indel markers and MSI-markers, respectively), T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES). As is clear from the above, detection of some of the individual additional biomarkers can be shared by different biomarker signatures.

Further aspects of the invention relate to using immunotherapy or immunogenic therapy in patients that are most likely to respond to the immunotherapy or immunogenic therapy. As outlined herein, such decision can be made based on increased expression of one or more of the retrotransposons identified herein. A further aspect of the invention thus relates to immunotherapeutic or immunogenic agents for use in (a method of) treating a tumor, for use in (a method of) inhibiting tumor progression or tumor relapse, or for use in (a method of) inhibiting tumor metastasis; or relates to use of an immunotherapeutic or immunogenic agents for (use in formulating a medicament for) treating a tumor, for (use in formulating a medicament for) inhibiting tumor progression or tumor relapse, or for (use in formulating a medicament for) inhibiting tumor metastasis; comprising:
detecting, determining or measuring an increased expression level of at least one retrotransposon in a sample obtained from the subject having the tumor, wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), wherein all retrotransposons are as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein the increased expression level is relative to the expression level of the same retrotransposon in a control sample or compared to a standard value; and
administering a therapeutically effective amount of the immunotherapeutic or immunogenic agent to the subject if an increased expression level of at least one retrotransposon is detected.

In particular, and referring to all multiple options outlined above in relation to the methods of the invention which equally apply here, the expression level of at least 4 retrotransposons may be analysed and an increased expression level of at least 1 of the at least 4 retrotransposons may be detected relative to the expression level of the same retrotransposons in a control sample or compared to a standard value, wherein the increased expression level of the at least 1 retrotransposon is indicative for administering a therapeutically effective amount of the immunotherapeutic or immunogenic agent to the subject.

Alternatively, the invention relates to immunotherapeutic or immunogenic agents for use in (a method of) treating a tumor, for use in (a method of) inhibiting tumor progression or tumor relapse, or for use in (a method of) inhibiting tumor metastasis; or relates to use of an immunotherapeutic or immunogenic agents for (use in formulating a medicament for) treating a tumor, for (use in formulating a medicament for) inhibiting tumor progression or tumor relapse, or for (use in formulating a medicament for) inhibiting tumor metastasis; comprising:
detecting, determining or measuring the expression level of at least one retrotransposon in a sample obtained from the subject having the tumor, wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), wherein all retrotransposons are as defined in Table 3 or Table 5, and/or further selected from the (group of) retrotransposons (consisting of) IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5;
detecting an increased expression level of at least one retrotransposon in the sample compared to the expression level of the same retrotransposon in a control sample or compared to a standard value; and
administering a therapeutically effective amount of the immunotherapeutic or immunogenic agent to the subject if an increased expression level of at least one retrotransposon is detected.

In particular, and referring to all multiple options outlined above in relation to the methods of the invention which equally apply here, the expression level of at least 4 retrotransposons may be analysed and an increased expression level of at least 1 of the at least 4 retrotransposons may be detected relative to the expression level of the same retrotransposons in a control sample or compared to a standard value, wherein the increased expression level of the at least 1 retrotransposon is indicative for administering a therapeutically effective amount of the immunotherapeutic or immunogenic agent to the subject.

In one embodiment, the immunotherapeutic or immunogenic agents are for use in (a method of) treating a tumor, for use in (a method of) inhibiting tumor progression or tumor relapse, or for use in (a method of) inhibiting tumor metastasis; or relates to use of an immunotherapeutic or immunogenic agents for (use in formulating a medicament for) treating a tumor, for (use in formulating a medicament for) inhibiting tumor progression or tumor relapse, or for (use in formulating a medicament for) inhibiting tumor metastasis; comprising the steps as described above, but further including detecting/determining/measuring the expression level of or an increase in the expression level of at least one retrotransposon selected from the retrotransposons HERVE_a-int (chrY), HERVK14C-int (chrY), HERV17-int (chrY), and L1ME2 (chrY), wherein all retrotransposons are defined in Table 3.

In a further embodiment, the immunotherapeutic or immunogenic agents are for use in (a method of) treating a tumor, for use in (a method of) inhibiting tumor progression or tumor relapse, or for use in (a method of) inhibiting tumor metastasis; or relates to use of an immunotherapeutic or immunogenic agents for (use in formulating a medicament for) treating a tumor, for (use in formulating a medicament for) inhibiting tumor progression or tumor relapse, or for (use in formulating a medicament for) inhibiting tumor metastasis; comprising the steps as described above, may entail detecting determining/measuring the expression level of or a change in the expression level of at least one retrotransposon selected from (the group consisting of) HERV9-int/AluY (chr12), THE1D (chr4), or MIRb (chrX), as defined in Table 3 or Table 5.

In a further embodiment, the tumor is melanoma.

In a further aspect, the invention relates to use of a panel of retrotransposons in any of the above described methods of the invention, wherein the panel is comprising 2 to 62 retrotransposons. In particular these retrotransposons are selected from Table 3 and/or Table 5. Alternatively, the invention relates to a panel of retrotransposons for use in any of the above described methods of the invention, wherein the panel is comprising 2 to 62 retrotransposons. In particular these retrotransposons are selected from Table 3 and/or Table 5. More details about the possible number of retrotransposons in the panel can be found in the relevant embodiments to the methods of the invention.

In a further aspect, the invention is relating to kits for use in any of the above described methods of the invention, wherein such kits are comprising the tools to detect the expression level of at least one retrotransposon, such as of 2 to 62 retrotransposons. In particular these retrotransposons are selected from Table 3 and/or Table 5. More details about the possible number of retrotransposons can be found in the relevant embodiments to the methods of the invention. In one embodiment, such kits further include the tools for detecting/determining/assessing/assaying/measuring the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES). In one specific embodiment, such kits are including the tools for detecting the status of at most 1000 markers, at most 950 markers, at most 900 markers, at most 850 markers, at most 800 markers, at most 750 markers, at most 700 markers, at most 650 markers, at most 600 markers, at most 550 markers, at most 500 markers, at most 450 markers, at most 400 markers, at most 350 markers, at most 300 markers, at most 250 markers, or at most 225, 200, 175, 150, 125, 111, 110, 105, 100, 95, 90, 85, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or at most 5 markers; in any case including at least one selected retrotransposon marker from the retrotransposon markers identified herein. Alternatively, such kits are including the tools for detecting the status of 2 to 10 markers, of 2 to 20 markers, of 2 to 50 markers, of 2 to 30 markers, of 2 to 50 markers, of 2 to 60 markers, of 2 to 70 markers, of 2 to 80 markers, of 2 to 90 markers, of 2 to 100 markers, of 2 to 150 markers, of 2 to 200 markers, of 2 to 300 markers, of 2 to 400 markers, of 2 to 500 markers, of 2 to 600 markers, of 2 to 700 markers, of 2 to 800 markers, of 2 to 900 markers, or of 2 to 1000 markers; in any case including at least one selected retrotransposon marker from the retrotransposon markers identified herein.

In particular, the tools of a kit of the invention comprise, besides optionally e.g. reagents, enzymes, reaction vessels and kit inserts, oligonucleotides capable of detecting the status of an envisaged biomarker. In particular, the oligonucleotides comprise a sequence specifically hybridizing to said biomarker or in the immediate vicinity of said biomarker. In particular, the oligonucleotide is comprising least one modified or non-naturally occurring nucleotide. Further in particular, the oligonucleotide may be part of a primer and probe set, of which set at least one primer or probe is comprising a sequence specifically hybridizing to the envisaged biomarker or in the immediate vicinity of said biomarker. Such kits can alternatively comprise a multi-membered set of oligonucleotides, wherein each member of the set comprises at least one modified or non-naturally occurring nucleotide and a sequence specifically hybridizing to one of the biomarkers or in the immediate vicinity of said biomarker. Such kits can alternatively comprise a plurality of separate primer and probe sets, wherein each set is comprising a primer or probe comprising of which at least one of the primer or probe is comprising a modified or non-naturally occurring nucleotide, and wherein each set comprises a primer or probe of which at least one of the primer or probe is comprising a sequence specifically hybridizing to one of the biomarkers or in the immediate vicinity of said biomarker. A non-naturally occurring nucleotide may be a nucleotide that is chemically different from a nucleotide present in a living cell (such as a labelled nucleotide), or may be a chemically naturally occurring nucleotide but which is mutated relative to the natural target nucleic acid on which the oligonucleotide is specifically hybridizing.

Tumor, Cancer, Neoplasm

The terms tumor and cancer are sometimes used interchangeably but can be distinguished from each other. A tumor refers to “a mass” which can be benign (more or less harmless) or malignant (cancerous).

A cancer is a threatening type of tumor. A tumor is sometimes referred to as a neoplasm: an abnormal cell growth, usually faster compared to growth of normal cells. Benign tumors or neoplasms are nonmalignant/non-cancerous, are usually localized and usually do not spread/metastasize to other locations. Because of their size, they can affect neighboring organs and may therefore need removal and/or treatment. A cancer, malignant tumor or malignant neoplasm is cancerous in nature, can metastasize, and sometimes re-occurs at the site from which it was removed (relapse). The initial site where a cancer starts to develop gives rise to the primary cancer. When cancer cells break away from the primary cancer (“seed”), they can move (via blood or lymph fluid) to another site even remote from the initial site. If the other site allows settlement and growth of these moving cancer cells, a new cancer, called secondary cancer, can emerge (“soil”). The process leading to secondary cancer is also termed metastasis, and secondary cancers are also termed metastases. For instance, liver cancer can arise as primary cancer, but can also be a secondary cancer originating from a primary breast cancer, bowel cancer or lung cancer; some types of cancer show an organ-specific pattern of metastasis. Most cancer deaths are in fact caused by metastases, rather than by primary tumors (Chambers et al. 2002, Nature Rev Cancer 2:563-572).

Sample, Biological Sample

A biological sample, or shortly sample, as referred to herein is any sample taken from a mammal having a tumor that can serve as source of retrotransposon detection. Such biological samples include tumor samples (such as obtained upon tumor biopsy), a bodily fluid sample or tumor exosomes from a mammal having a tumor. The biological sample thus in general is a biological sample suspected to comprise/of comprising tumor retrotransposon material or a biological sample comprising tumor retrotransposon material. The tumor retrotransposon material can be RNA and/or DNA. Except for tumor samples, (a pool of) corresponding biological samples of healthy mammals can be used as control or reference.

Tumors are known to shed fragments of genomic DNA known as circulating tumor DNA or ctDNA (which is part of the circulating free DNA or cfDNA). For example, a bodily fluid sample can comprise, without limitation, bodily fluid, whole blood, serum, plasma, synovial fluid, lymphatic fluid, ascites fluid, interstitial or extracellular fluid, the fluid in spaces between cells, including gingival crevicular fluid, cerebrospinal fluid, saliva, mucous, sputum, phlegm, smegma, seminal fluid, ejaculate, sweat, tears, urine, fluid from nasal brushings, colonic washing fluid, fluid from a pap smear, vaginal fluid, vaginal flushing fluid, fluid from a hydrocele, pleural fluid, bronchoalveolar lavage fluid, discharge fluid from the nipple, aspiration fluid from a part of the body, colostrum, breast milk, ventricular fluid, any other bodily fluids. A bodily fluid can include saliva, blood, or serum. Bodily fluids of a subject can comprise ctDNA when the subject is having a tumor or cancer.

Tumors are known to produce exosomes (small membrane vesicles or microvesicles of endocytic origin). Compared to normal cells, the release of such exosomes by tumor cells is often elevated, which results in elevated levels of tumor-derived exosomes in the peripheral circulation and in bodily fluids such as serum or plasma, ascites, urine, and pleural effusions. This has led to the proposal to use such exosomes in diagnosis of cancer or for cancer biomarker analysis (e.g. Taylor & Gercel-Taylor 2008, Gynecol Oncol 110:13-21, and references cited therein). Even brain tumors such as glioblastoma produce exosomes that can be isolated from serum (Skog et al. 2008, Nature Cell Biol 10:1470-1476). Urine was reported to harbor exosomes of e.g. prostate cancer; ascites to harbor exosomes of e.g. colorectal cancer; and pleural effusions to harbor exosomes of e.g. mesothelioma, lung cancer, breast cancer, and ovarian cancer (van der Pol et al. 2012, Pharmacol Rev 64:676-705 and references cited therein). Tumor exosomes were demonstrated to contain retrotransposon elements, more in particular retrotransposon RNA and, where reverse transcriptase is present in tumor exosomes, also retrotransposon (c)DNA (Balaj et al. 2011, Nature Comm 2:180). One way of enriching tumor exosomes prior to their analysis is ultracentrifugation of serum to form a pellet (e.g. Balaj et al. 2011, Nature Comm 2:180; Skog et al. 2008, Nature Cell Biol 10:1470-1476). When a specific target is present on the exosomes, then cell sorting technology can be used; epithelial tumors were for instance shown to produce exosomes containing epithelial cell adhesion molecule (EpCAM) and were purified from serum by magnetic activated cell sorting using anti-EpCAM coupled to magnetic beads (e.g. Taylor & Gercel-Taylor 2008, Gynecol Oncol 110:13-21).

Immunotherapy and Immunogenic Therapy

Immunotherapy in general is defined as a treatment that uses the body's own immune system to help fight a disease, more specifically cancer in the context of the current invention. Immunotherapeutic treatment as used herein refers to the reactivation and/or stimulation and/or reconstitution of the immune response of a mammal towards a condition such as a tumor, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumor or anti-neoplasm immune response; adaptive immune response to the tumor, cancer or neoplasm). Immunotherapeutic agents of particular interest include immune checkpoint inhibitors (such as anti-PD-1, anti-PD-L1 or anti-CTLA-4 antibodies), bispecific antibodies bridging a cancer cell and an immune cell, dendritic cell vaccines, Immunotherapy is a promising new area of cancer therapeutics and several immunotherapies are being evaluated preclinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumor progression. An overview of clinical developments in the field of immune checkpoint therapy is given by Fan et al. 2019 (Oncology Reports 41:3-14). Monoclonal antibodies targeting and inhibiting PD-1 include pembrolizumab, nivolumab, and cemiplimab. Monoclonal antibodies targeting and inhibiting PD-L1 include atezolizumab, avelumab, and durvalumab. Monoclonal antibodies targeting and inhibiting CTLA-4 include ipilimumab. Combinatorial cancer treatments that include chemotherapies can achieve higher rates of disease control by impinging on distinct elements of tumor biology to obtain synergistic antitumor effects. It is now accepted that certain chemotherapies can increase tumor immunity by inducing immunogenic cell death and by promoting escape in cancer immunoediting, such therapies are therefore called immunogenic therapies as they provoke an immunogenic response. Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187). Other forms of immunotherapy include chimeric antigen receptor (CAR) T-cell therapy in which allogenic T-cells are adapted to recognize a tumoral neo-antigen and oncolytic viruses preferentially infecting and killing cancer cells. Treatment with RNA, e.g. encoding MLKL, is a further means of provoking an immunogenic response (Van Hoecke et al. 2018, Nat Commun 9:3417), as well as vaccination with neo-epitopes (Brennick et al. 2017, Immunotherapy 9:361-371).

Gene Expression Level

The term “level of expression” or “expression level” generally refers to the amount of an expressed biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) are also regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs, long non-coding RNA, microRNA or miRNA).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who do not have the disease or disorder (e.g., cancer), an internal control (e.g., a housekeeping biomarker), a median expression level of the biomarker in samples from a group/population of patients, or relative to an expression level of the biomarker in samples taken before onset of a certain therapy.

The term “detection” includes any means of detecting, including direct and indirect detection. The term “biomarker” as used herein refers to an indicator molecule or set of molecules (e.g., predictive, diagnostic, and/or prognostic indicator), which can be detected in a sample. The biomarker may be a predictive biomarker and serve as an indicator of the likelihood of sensitivity or benefit of a patient having a particular disease or disorder (e.g., a proliferative cell disorder (e.g., cancer)) to treatment. Biomarkers include, but are not limited to, polynucleotides (e.g., DNA and/or RNA (e.g., mRNA)), polynucleotide copy number alterations (e.g., DNA copy numbers), polypeptides, polypeptide and polynucleotide modifications (e.g., post-translational modifications, nucleotide substitutions, nucleotide insertions or deletions (indels)), carbohydrates, and/or glycolipid-based molecular markers. In some embodiments, a biomarker is a gene. The “amount” or “level” of a biomarker, as used herein, is a detectable level in a biological sample. These can be measured by methods known to one skilled in the art and also disclosed herein.

Any gene detection or gene expression detection method is starting from an analyte nucleic acid (i.e. the nucleic acid of interest (which does not necessarily need to be the whole nucleic acid of interest, parts of such nucleic acids can suffice for determining expression) and of which the amount is to be determined) and may be defined as comprising one or more of, for instance,
a step of isolating RNA from a biological sample (wherein a fraction of the isolated RNA is the analyte strand).
a step of reverse transcribing the RNA obtained from the biological sample into DNA;
a step of amplifying the isolated DNA; and/or
a step of quantifying the isolated RNA, the DNA obtained after reverse transcription, or the amplified DNA.

In case an amplified DNA is quantified, this quantification step can be performed concurrent with the amplification of the DNA, or is performed after the amplification of the DNA.

The quantification of gene expression or the determination of gene expression levels may be based on at least one of an amplification reaction, a sequencing reaction, a melting reaction, a hybridization reaction or a reverse hybridization reaction.

Detection and Quantification of Gene Expression

The invention covers methods for detecting the presence of nucleic acids corresponding to one or more retrotransposon(s) as defined herein in a biological sample and/or methods for determining or detecting the expression level of one or more retrotransposon(s) as defined herein, wherein said methods comprise the step of detecting the presence of a retrotransposon of interest nucleic acid or expression level of a retrotransposon of interest. In any of these methods the detection can comprise a step such as a nucleic acid amplification reaction, a nucleic acid sequencing reaction, a melting reaction, a hybridization reaction to a nucleic acid, or a reverse hybridization reaction to a nucleic acid, or a combination of such steps.

Often one or more artificial, man-made, non-naturally occurring oligonucleotide is used in such method. In particular, such oligonucleotides can comprise besides ribonucleic acid monomers or deoxyribonucleic acid monomers: one or more modified nucleotide bases, one or more modified nucleotide sugars, one or more labelled nucleotides, one or more peptide nucleic acid monomers, one or more locked nucleic acid monomers, the backbone of such oligonucleotide can be modified, and/or non-glycosidic bonds may link two adjacent nucleotides. Such oligonucleotides may further comprise a modification for attachment to a solid support, e.g., an amine-, thiol-, 3-′propanolamine or acrydite-modification of the oligonucleotide, or may comprise the addition of a homopolymeric tail (for instance an oligo(dT)-tail added enzymatically via a terminal transferase enzyme or added synthetically) to the oligonucleotide. If said homopolymeric tail is positioned at the 3′-terminus of the oligonucleotide or if any other 3′-terminal modification preventing enzymatic extension is incorporated in the oligonucleotide, the priming capacity of the oligonucleotide can be decreased or abolished. Such oligonucleotides may also comprise a hairpin structure at either end. Terminal extension of such oligonucleotide may be useful for, e.g., specifically hybridizing with another nucleic acid molecule (e.g. when functioning as capture probe), and/or for facilitating attachment of said oligonucleotide to a solid support, and/or for modification of said tailed oligonucleotide by an enzyme, ribozyme or DNAzyme. Such oligonucleotides may be modified in order to detect (the levels of) a target nucleotide sequence and/or to facilitate in any way such detection. Such modifications include labelling with a single label, with two different labels (for instance two fluorophores or one fluorophore and one quencher), the attachment of a different ‘universal’ tail to two probes or primers hybridizing adjacent or in close proximity to each other with the target nucleotide sequence, the incorporation of a target-specific sequence in a hairpin oligonucleotide (for instance Molecular Beacon-type primer), the tailing of such a hairpin oligonucleotide with a ‘universal’ tail (for instance Sunrise-type probe and Amplifluor™-type primer). A special type of hairpin oligonucleotide incorporates in the hairpin a sequence capable of hybridizing to part of the newly amplified target DNA. Amplification of the hairpin is prevented by the incorporation of a blocking nonamplifiable monomer (such as hexethylene glycol). A fluorescent signal is generated after opening of the hairpin due to hybridization of the hairpin loop with the amplified target DNA. This type of hairpin oligonucleotide is known as scorpion primers (Whitcombe et al. 1999, Nat Biotechnol 17:804-807). Another special type of oligonucleotide is a padlock oligonucleotide (or circularizable, open circle, or C-oligonucleotide) that are used in RCA (rolling circle amplification). Such oligonucleotides may also comprise a 3′-terminal mismatching nucleotide and/or, optionally, a 3′-proximal mismatching nucleotide, which can be particularly useful for performing polymorphism-specific PCR and LCR (ligase chain reaction) or any modification of PCR or LCR. Such oligonucleotide may can comprise or consist of at least and/or comprise or consist of up to 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or more contiguous nucleotides.

The analyte nucleic acid, in particular the analyte nucleic acid of a retrotransposon of interest can be any type of nucleic acid, which will be dependent on the manipulation steps (such as isolation and/or purification and/or duplication, multiplication or amplification) applied to the nucleic acid of the gene of interest in the biological sample; as such it can be DNA, RNA, cDNA, may comprise modified nucleotides, or may be hybrids of DNA and/or RNA and/or modified nucleotides, and can be single- or double-stranded or may be a triplex-forming nucleic acid.

The artificial, man-made, non-naturally occurring oligonucleotide(s) as applied in the above detection methods can be probe(s) or a primer(s), or a combination of both.

A probe capable of specifically hybridizing with a target nucleic acid is an oligonucleotide mainly hybridizing to one specific nucleic acid sequence in a mixture of many different nucleic acid sequences. Specific hybridization is meant to result, upon detection of the specifically formed hybrids, in a signal-to-noise ratio (wherein the signal represents specific hybridization and the noise represents unspecific hybridization) sufficiently high to enable unambiguous detection of said specific hybrids. In a specific case specific hybridization allows discrimination of up to a single nucleotide mismatch between the probe and the target nucleic acids. Conditions allowing specific hybridization generally are stringent but can obviously be varied depending on the complexity (size, GC-content, overall identity, etc.) of the probe(s) and/or target nucleic acid molecules. Specificity of a probe in hybridizing with a nucleic acid can be improved by introducing modified nucleotides in said probe.

A primer capable of directing specific amplification of a target nucleic acid is the at least one oligonucleotide in a nucleic acid amplification reaction mixture that is required to obtain specific amplification of a target nucleic acid. Nucleic acid amplification can be linear or exponential and can result in an amplified single nucleic acid of a single- or double-stranded nucleic acid or can result in both strands of a double-stranded nucleic acid. Specificity of a primer in directing amplification of a nucleic acid can be improved by introducing modified nucleotides in said primer. The fact that a primer does not have to match exactly with the corresponding template or target sequence to warrant specific amplification of said template or target sequence is amply documented in literature (for instance: Kwok et al. 1990, Nucl Acids Res 18:999-1005. Primers as short as 8 nucleotides in length have been applied successfully in directing specific amplification of a target nucleic acid molecule (e.g. Majzoub et al. 1983, J Biol Chem 258:14061-14064).

A nucleotide is meant to include any naturally occurring nucleotide as well as any modified nucleotide wherein said modification can occur in the structure of the nucleotide base (modification relative to A, T, G, C, or U) and/or in the structure of the nucleotide sugar (modification relative to ribose or deoxyribose). Any of the modifications can be introduced in a nucleic acid or oligonucleotide to increase/decrease stability and/or reactivity of the nucleic acid or oligonucleotide and/or for other purposes such as labelling of the nucleic acid or oligonucleotide. Modified nucleotides include phophorothioates, alkylphophorothioates, methylphosphonate, phosphoramidate, peptide nucleic acid monomers and locked nucleic acid monomers, cyclic nucleotides, and labelled nucleotides (i.e. nucleotides conjugated to a label which can be isotopic (<32>P, <35>S, etc.) or non-isotopic (biotin, digoxigenin, phosphorescent labels, fluorescent labels, fluorescence quenching moiety, etc.)). Other modifications are described higher (see description on oligonucleotides).

Nucleotide acid amplification is meant to include all methods resulting in multiplication of the number of a target nucleic acid. Nucleotide sequence amplification methods include the polymerase chain reaction (PCR; DNA amplification), strand displacement amplification (SDA; DNA amplification), transcription-based amplification system (TAS; RNA amplification), self-sustained sequence replication (3SR; RNA amplification), nucleic acid sequence-based amplification (NASBA; RNA amplification), transcription-mediated amplification (TMA; RNA amplification), Qbeta-replicase-mediated amplification and run-off transcription. During amplification, the amplified products can be conveniently labeled either using labeled primers or by incorporating labeled nucleotides.

The most widely spread nucleotide sequence amplification technique is PCR. The target DNA is exponentially amplified. Many methods rely on PCR including AFLP (amplified fragment length polymorphism), IRS-PCR (interspersed repetitive sequence PCR), iPCR (inverse PCR), RAPD (rapid amplification of polymorphic DNA), RT-PCR (reverse transcription PCR) and real-time PCR. RT-PCR can be performed with a single thermostable enzyme having both reverse transcriptase and DNA polymerase activity (Myers et al. 1991, Biochem 30:7661-7666). Alternatively, a single tube-reaction with two enzymes (reverse transcriptase and thermostable DNA polymerase) is possible (Cusi et al. 1994, Biotechniques 17:1034-1036).

Solid phases, solid matrices or solid supports on which molecules, e.g., nucleic acids, analyte nucleic acids and/or oligonucleotides as described hereinabove, may be bound (or captured, absorbed, adsorbed, linked, coated, immobilized; covalently or non-covalently) comprise beads or the wells or cups of microtiter plates, or may be in other forms, such as solid or hollow rods or pipettes, particles, e.g., from 0.1 μm to 5 mm in diameter (e.g. “latex” particles, protein particles, or any other synthetic or natural particulate material), microspheres or beads (e.g. protein A beads, magnetic beads). A solid phase may be of a plastic or polymeric material such as nitrocellulose, polyvinyl chloride, polystyrene, polyamide, polyvinylidene fluoride or other synthetic polymers. Other solid phases include membranes, sheets, strips, films and coatings of any porous, fibrous or bibulous material such as nylon, polyvinyl chloride or another synthetic polymer, a natural polymer (or a derivative thereof) such as cellulose (or a derivative thereof such as cellulose acetate or nitrocellulose). Fibers or slides of glass, fused silica or quartz are other examples of solid supports. Paper, e.g., diazotized paper may also be applied as solid phase. Clearly, molecules such as nucleic acids, analyte nucleic acids and/or oligonucleotides as described hereinabove, may be bound, captured, absorbed, adsorbed, linked or coated to any solid phase suitable for use in hybridization assay (irrespective of the format, for instance capture assay, reverse hybridization assay, or dynamic allele-specific hybridization (DASH)). Said molecules, such as nucleic acids, analyte nucleic acids and/or oligonucleotides as described hereinabove, can be present on a solid phase in defined zones such as spots or lines. Such solid phases may be incorporated in a component such as a cartridge of e.g. an assay device. Any of the solid phases described above can be developed, e.g. automatically developed in an assay device.

Quantification of amplified DNA can be performed concurrent with or during the amplification. Techniques include real-time PCR or (semi-)quantitative polymerase chain reaction (qPCR). One common method includes measurement of a non-sequence specific fluorescent dye (e.g. SYBR Green) intercalating in any double-stranded DNA. Quantification of multiple amplicons with different melting points can be followed simultaneously by means of following or analyzing the melting reaction (melting curve analysis or melt curve analysis; which can be performed at high resolution, see, e.g. Wittwer et al. 2003, Clin Chem 843-860; an alternative method is denaturing gel gradient electrophoresis, DGGE; both methods were compared in e.g. Tindall et al. 2009, Hum Mutat 30:857-859).

Another common method includes measurement of sequence-specific labelled probe bound to its complementary sequence; such probe also carries a quencher and the label is only measurable upon exonucleolytic release from the probe (hydrolysis probes such as TaqMan probes) or upon hybridization with the target sequence (hairpin probes such as molecular beacons which carry an internally quenched fluorophore whose fluorescence is restored upon unfolding the hairpin). This latter method allows for multiplexing by e.g. using mixtures of probes each tagged with a different label e.g. fluorescing at a different wavelength.

Exciton-controlled hybridization-sensitive fluorescent oligonucleotide (ECHO) probes also allow for multiplexing. The hybridization-sensitive fluorescence emission of ECHO probes and the further modification of probes have made possible multicolor RNA imaging in living cells and facile detection of gene polymorphisms (Okamoto 2011, Chem Soc Rev, 40:5815-5828).

Other methods of quantifying expression include SAGE (Serial Analysis of Gene Expression) and MPSS (Massively Parallel Signature Sequencing), each involving reverse-transcription of RNA.

With “assaying” or “determining” or “detecting” and the like (e.g. assessing, measuring) is meant that a biological sample, suspected of comprising a target nucleic acid (such as a nucleic acid of interest as described herein), is processed as to generate a readable signal in case the target nucleic acid is actually present in the biological sample. Such processing may include, as described above, a step of producing an analyte nucleic acid. Simple detection of a produced readable signal indicates the presence of a target or analyte nucleic acid in the biological sample. When in addition the amplitude of the produced readable signal is determined, this allows for quantification of levels of a target or analyte nucleic acid as present in a biological sample.

In particular, the readable signal may be a signal-to-noise ratio (wherein the signal represents specific detection and the noise represents unspecific detection) of an assay optimized to yield signal-to-noise ratios sufficiently high to enable unambiguous detection and/or quantification of the target nucleic acid. The noise signal, or background signal, can be determined e.g. on biological samples not comprising the target or analyte nucleic acid of interest, e.g. control samples, or comprising the required reference level of the target or analyte nucleic acid of interest, e.g. reference samples. Such noise or background signal may also serve as comparator value for determining an increase or decrease of the level of a target or analyte nucleic acid in the biological sample, e.g. in a biological sample taken from a subject suffering from a disease or disorder, further e.g. before start of a treatment and during treatment.

The readable signal may be produced with all required components in solution or may be produced with some of the required components in solution and some bound to a solid support. Said signals include, e.g., fluorescent signals, (chemi)luminescent signals, phosphorescence signals, radiation signals, light or color signals, optical density signals, hybridization signals, mass spectrometric signals, spectrometric signals, chromatographic signals, electric signals, electronic signals, electrophoretic signals, real-time PCR signals, PCR signals, LCR signals, Invader-assay signals, sequencing signals (by any method such as Sanger dideoxy sequencing, pyrosequencing, 454 sequencing, single-base extension sequencing, sequencing by ligation, sequencing by synthesis, “next-generation” sequencing (NGS)(van Dijk et al. 2014, Trends Genet 30:418-426)), melting curve signals etc. An assay may be run automatically or semi-automatically in an assay device. In view of its relatively low costs compared to e.g. very costly cancer therapies, NGS is finding its way to routine clinical care (Ratner 2018, Nature Biotechnol 36:484).

Specific hybridization of an oligonucleotide (whether or not comprising one or more modified nucleotides) to its target sequence is to be understood to occur under stringent conditions as generally known in the art (e.g. Sambrook et al. 1989. Molecular Cloning. A laboratory manual. CSHL Press). However, depending to the hybridization solution (SSC, SSPE, etc.), oligonucleotides should be hybridized at their appropriate temperature in order to attain sufficient specificity. In order to allow hybridization to occur, the target nucleic acid molecules are generally thermally, chemically (e.g. by NaOH) or electrochemically denatured to melt a double strand into two single strands and/or to remove hairpins or other secondary structures from single stranded nucleic acids. The stringency of hybridization is influenced by conditions such as temperature, salt concentration and hybridization buffer composition. High stringency conditions for hybridization include high temperature and/or low salt concentration (salts include NaCl and Na3-citrate) and/or the inclusion of formamide in the hybridization buffer and/or lowering the concentration of compounds such as SDS (detergent) in the hybridization buffer and/or exclusion of compounds such as dextran sulfate or polyethylene glycol (promoting molecular crowding) from the hybridization buffer. Conventional hybridization conditions are described in e.g. Sambrook et al. 1989 (Molecular Cloning. A laboratory manual. CSHL Press) but the skilled craftsman will appreciate that numerous different hybridization conditions can be designed in function of the known or the expected homology and/or length of the nucleic acid sequence. Generally, for hybridizations with DNA oligonucleotides without formamide, a temperature of 68DEG C., and for hybridization with formamide, 50% (v/v), a temperature of 42DEG C. is recommended. For hybridizations with oligonucleotides, the optimal conditions (formamide concentration and/or temperature) depend on the length and base composition of the probe and must be determined individually. In general, optimal hybridization for oligonucleotides of about 10 to 50 bases in length occurs approximately 5 DEG C below the melting temperature for a given duplex. Incubation at temperatures below the optimum may allow mismatched sequences to hybridize and can therefor result in reduced specificity. When using RNA oligonucleotides with formamide (50% v/v) it is recommend to use a hybridization temperature of 68DEG C. for detection of target RNA and of 50DEG C. for detection of target DNA. Alternatively, a high SDS hybridization solution can be utilized (Church et al. 1984, Proc Natl Acad Sci USA 81:1991-1995). The specificity of hybridization can furthermore be ensured through the presence of a crosslinking moiety on the oliogonucleotide (e.g. Huan et al. 2000, Biotechniques 28: 254-255; WO00/14281). Said crosslinking moiety enables covalent linking of the oligonucleotide with the target nucleotide sequence and hence allows stringent washing conditions. Such a crosslinking oliogonucleotide can furthermore comprise another label suitable for detection/quantification of the oligonucleotide hybridized to the target.

As outlined in the Examples, RPKM is often used as measure for expression. FPKM (Fragments Per Kilobase Million) is very similar to RPKM RPKM (Reads Per Kilobase Million); whereas RPKM was designed for single-end RNA-seq (every read corresponded to a single sequenced fragment), FPKM was designed for paired-end RNA-seq. With paired-end RNA-seq, two reads can correspond to a single fragment, or, if one read in the pair did not map, one read can correspond to a single fragment. The only difference between RPKM and FPKM is that FPKM takes into account that two reads can map to one fragment (and so it doesn't count this fragment twice). When using RNA-seq, reporting or results often is in RPKM (Reads Per Kilobase Million) or FPKM (Fragments Per Kilobase Million). Whatever metric used (another alternative for example is TPM (Transcripts Per Kilobase Million)), such metric is attempting to normalize for sequencing depth and gene length and provide a measure for quantifying transcript levels/gene expression/expression units.

Determination of DNA Methylation

Assays for DNA methylation analysis have been reviewed by e.g. Laird 2010 (Nat Rev Genet 11:191-203). The main principles of possible sample pre-treatment involve enzyme digestion (relying on restriction enzymes sensitive or insensitive to methylated nucleotides), affinity enrichment (involving e.g. chromatin immunoprecipitation, antibodies specific for 5MeC, methyl-binding proteins), sodium bisulfite treatment (converting an epigenetic difference into a genetic difference) followed by analytical steps (locus-specific analysis, gel-based analysis, array-based analysis, next-generation sequencing-based analysis) optionally combined in a comprehensible matrix of assays. Laird 2010 is providing a plethora of bioinformatic resources useful in DNA methylation analysis which can be applied by the skilled person as guiding principles, when wishing to analyze the methylation status of up to about 100 CpGs in a sample, with assays such as MethyLight, EpiTYPER, MSP, COBRA, Pyrosequencing, Southern blot and Sanger BS appearing to be the most suitable assays. This guidance does, however, not take into account that assays with higher coverage can be adapted towards lower coverage. For example, design of custom DNA methylation profiling assays covering up to 96 or up to 384 individual regions is possible e.g. by using the VeraCode® technology provided by IIlumina® (compared to the 450K DNA methylation array covering approximately 480000 individual CpGs). Another such adaptation for instance is enrichment of genome fractions comprising methylation regions of interest which is possible by e.g. hybridization with bait sequences. Such enrichment may occur before bisulfite conversion (e.g. customized version of the SureSelect Human Methyl-Seq from Agilent) or after bisulfite conversion (e.g. customized version of the SeqCap Epi CpGiant Enrichment Kit from Roche). Such targeted enrichment can be considered as a further modification/simplification of RRBS (Reduced Representation Bisulfite Sequencing).

The MethyLight assay is a high-throughput quantitative or semi-quantitative methylation assay that utilizes fluorescence-based real-time PCR (e.g., TagMan®) that requires no further manipulations after the PCR step (Eads et al. 2000, Nucleic Acids Res 28:e32). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs at the level of the amplification process, at the level of the probe detection process, or at both levels. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites or with oligonucleotides covering potential methylation sites.

The EpiTYPER assay involves many steps including gene-specific amplification of bisulfite-converted genomic DNA, in vitro transcription of the amplified DNA, uranil-specific cleavage of transcribed RNA, and MALDI-TOF analysis of the RNA fragments. The EpiTYPER software finally distinguishes between methylated and non-methylated cytosine in the genomic DNA.

Methylation-specific PCR (MSP) refers to the methylation assay as described by Herman et al. 1996 (Proc Natl Acad Sci USA 93:9821-9826), and by U.S. Pat. No. 5,786,146. MSP (methylation-specific PCR) allows for assessing the methylation status of virtually any group of CpG sites within a CpG island, independent of the use of methylation-sensitive restriction enzymes. Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG. Variations of MSP include Methylation-sensitive Single Nucleotide Primer Extension (Ms-SNuPE; Gonzalgo & Jones 1997, Nucleic Acids Res 25:2529-2531). Another variation, however including restriction enzyme digestion instead of bisulfite modification as sample pretreatment, is Methylation-Sensitive Arbitrarily-Primed Polymerase Chain Reaction (MS AP-PCR; Gonzalgo et al. 1997, Cancer Research 57:594-599).

Combined Bisulfite Restriction Analysis (COBRA) refers to the methylation assay described by Xiong & Laird 1997 (Nucleic Acids Res 25:2532-2534). COBRA analysis is a quantitative methylation assay useful for determining DNA methylation levels at specific loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by bisulfite treatment. PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Sanger BS is the original way of analysis of bisulfite-treated DNA: gel electrophoresis-based Sanger sequencing of cloned PCR products from single loci (Frommer et al. 1992, Proc Natl Acad Sci USA 89:1827-1831). A technique such as pyrosequencing is similar to Sanger BS and obviates the need of gel electrophoresis; it, however, requires other specialized equipment (e.g. Pyromark instrument). Sequencing approaches are still applied, especially with the emergence of next-generation sequencing (NGS) platforms. Southern blot analysis of DNA methylation depends on methyl-sensitive restriction enzymes (e.g. Moore 2001, Methods Mol Biol 181:193-201).

Other assays to determine CpG methylation include the HeavyMethyl (HM) assay (Cottrell et al. 2004, Nucleic Acids Res 32, e10; WO2004113567), Methylated CpG Island Amplification (MCA; Toyota et al. 1999, Cancer Res 59:2307-12; WO 00/26401), Reduced Representation Bisulfite Sequencing (RRBS; e.g. Meissner et al. 2005, Nucleic Acids Res 33: 5868-5877), Quantitative Allele-specific Real-time Target and Signal amplification (QuARTS; e.g. WO2012067830), and assays described in Laird et al. 2010 (Nat Rev Genet 11:191-203) and in Kurdyukov & Bullock 2016 (Biology 5(1), pii: E3).

Bisulfite reagents convert unmethylated cytosine moieties in DNA into uracil moieties. Drawbacks of such bisulfite reagents are DNA degradation (although perhaps only relevant for long DNA molecules) and lack of complete conversion. Other methods to convert unmethylated cytosine to uracil include TET-assisted bisulfite sequencing (TAB-Seq; involving ten-eleven translocation (TET) enzyme; Yu et al. 2012, Cell 149:1368-1380) and oxidative bisulfite sequencing (oxBS; involving potassium perruthenate; Booth et al. 2012, Science 336:934-937).

An alternative method relies on conversion of 5-methyl-cytosine (5mC) and 5-hydroxy-methyl-cytosine (5hmC) to dihydrouracil (DHU), leaving unmethylated cytosines unaffected. Such method is known as ten-eleven translocation (TET)-assisted pyridine borane sequencing or TAPS. First, 5mC and 5hmC are oxidized by TET enzymes, resulting in conversion to 5-carboxyl-cytosine (5caC). 5caC moieties are then reduced by pyridine borane or 2-picoline borane, resulting in conversion to DHU. Upon duplication or amplification, DHU is converted to thymine (methylated cytosine to thymine conversion) in the duplicated or amplified DNA or RNA. Selective conversion of 5mC (and not 5hmC) to DHU is possible by protecting 5hmC from TET-oxidation by means of adding a glucose to 5hmC (to produce 5gmC) by means of a beta-glucosyltransferase (method referred to as TAPSβ); selective conversion of 5hmC (and not 5mC) is possible by oxidizing 5hmC by means of potassium perruthenate to produce 5-formyl-cytosine (5fmC) and subsequent borane reduction to convert 5fmC to DHU (method referred to as chemical-assisted pyridine borane sequencing or CAPS) (Liu et al. 2019, Nat Biotechnol 37:424-429).

Treatment/Therapeutically Effective Amount

“Treatment”/“treating” refers to any rate of reduction, delaying or retardation of the progress of the disease or disorder, or a single symptom thereof, compared to the progress or expected progress of the disease or disorder, or singe symptom thereof, when left untreated. This implies that a therapeutic modality on its own may not result in a complete or partial response (or may even not result in any response), but may, in particular when combined with other therapeutic modalities, contribute to a complete or partial response (e.g. by rendering the disease or disorder more sensitive to therapy). More desirable, the treatment results in no/zero progress of the disease or disorder, or singe symptom thereof (i.e. “inhibition” or “inhibition of progression”), or even in any rate of regression of the already developed disease or disorder, or singe symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival, duration of response, and/or quality of life. The term “effective amount” refers to the dosing regimen of the agent (e.g. antagonist as described herein) or composition comprising the agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the mammal or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a mammal in need thereof, i.e., harboring a tumor, cancer or neoplasm in need of treatment. In general a (therapeutically) effective amount of (a) therapeutic compound(s) is administered to the mammal in need thereof in order to obtain the described clinical response(s). “Administering” means any mode of contacting that results in interaction between an agent (e.g. a therapeutic compound) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

Computer/Computer System

A computer or computer system as mentioned herein may utilize one or more subsystems. A computer or computer system may be a single computer apparatus comprising the one or more subsystems (e.g. internal components), or may be multiple computers or multiple computer apparatuses each being a subsystem, and optionally, each comprising one or more own subsystems. Desktops, laptops, mainframe servers, tablets, mobile phones etc. all are computers or computer systems. The subsystems are usually interconnected and include a (central) processor (single-core processor, multi-core processor on a same integrated chip, or multiple processing units on a single circuit board or networked) capable of executing instructions, an input/output (I/O) controller, and a storage device (external, internal, peripheral, cloud, any medium readable by a computer or computer system). Input devices include keyboards, scanners, a computer mouse, camera, microphone, etc. In particular, the input device is a data collection or data generating device (which by itself may comprise a computer or computer system), such as a polynucleotide sequencing device (whether automated or not). Collected or generated data are fed to a computer or computer system designed to analyze the collected or generated data; this may be an ordinary computer system on which data analyzing software is installed (on a storage device) or which is capable of accessing data analyzing software (e.g. installed in or transmitted from a network) and whereby the processor of the computer system is instructed by the data analysis software on how to process the collected or generated data fed to the computer system, and how to display these via a display adapter to an output device. Output devices are further subsystems and comprise printers, monitors, computer readable medium. Input and output devices are usually connected to a computer or computer system via input/output ports to one another or via a network.

The specific combination of hardware and software allows implementation of e.g. analysis of data generated by a polynucleotide sequencing device or expression analysis device. Different software packages (proprietary or open source) can be run on a computer or computer system to achieve the desired degree of data analysis. Output of one computerized data analysis can be the input of a subsequent computerized data analysis step, hence creating an analysis pipeline. Software components can be written in different codes (e.g. Java, C, C++, Swith, Perl, Python) as long as the computer processor is able to execute the functions of the software component.

The methods of the invention may be computer-implemented methods, or methods that are assisted or supported by a computer or by a computer system. For instance, information reflecting the analysis, determination, detection, presence or absence of DNA methylation, or of determining, detecting, assaying, assessing or analyzing biomarker expression or biomarker expression levels obtained from a sample is received by at least one first processor, and/or information reflecting the analysis, determination, detection, presence or absence of DNA methylation, or of determining, detecting, assaying, assessing or analyzing biomarker expression or biomarker expression levels obtained from a sample is provided in user readable format by at least one/another processor. The same or a further processor may be calculating a relative DNA methylation (such as relative to a control or standard), or a relative biomarker expression or biomarker expression level (such as relative to a control or standard) from the information received. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. Such computer-implemented methods (or such methods that are assisted or supported by a computer) may be provided as a kit or as part of a kit. The bioinformatics software required to perform (part of) the computer-implemented methods, i.e. a computer program product, may also be part of a kit, or may be provided as an individual product. A computer product may also consist of a computer readable medium which is storing any of the instructions, computer program, or bioinformatics software enabling a computer system to perform at least one of the analysis of the herein described methods and/or to perform at least one calculation (of DNA methylation or of biomarker expression or biomarker expression level) as described herein.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims The content of the documents cited herein are incorporated by reference.

The invention is further described in the paragraphs following hereafter.
1. A method of tumor analysis, the method comprising the step of detecting in a sample obtained from a subject having the tumor, a change in the expression level of at least one retrotransposon relative to the expression level of the same retrotransposon in a control sample or compared to a standard value, wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5.
2. A method of determining prior to or early after start of immunotherapy or of an immunogenic therapy the outcome of the immunotherapy or the immunogenic therapy, or of determining susceptibility to the immunotherapy or the immunogenic therapy of a tumor in a subject, comprising the step of detecting a change in the expression level of at least one retrotransposon in a sample obtained from the subject, and wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein an increased expression level of the retrotransposon in the sample relative to the expression level of the same retrotransposon in a control sample or compared to a standard value, is indicative of a positive outcome of the immunotherapy or the immunogenic therapy or is indicative of susceptibility of the tumor to the immunotherapy or the immunogenic therapy.
3. The method according to paragraph 2 wherein the expression level of at least 4 retrotransposons is determined and wherein an increase in expression level of at least 1 of the at least 4 retrotransposons is detected relative to the expression level of the same retrotransposons in a control sample or compared to a standard value, and wherein the increased expression level of the at least 1 retrotransposon is indicative of a positive outcome of the immunotherapy or the immunogenic therapy or is indicative of susceptibility of the tumor to the immunotherapy or the immunogenic therapy.
4. A method of determining response to immunotherapy or to immunogenic therapy of a tumor in a subject, comprising the step of detecting a change in the expression level of at least one retrotransposon in a sample obtained from the subject, and wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-1(chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein a decrease in the expression level of the retrotransposon in the sample relative to the expression level of the same retrotransposon in a sample obtained from the subject prior to immunotherapy or immunogenic therapy or in a sample obtained at an earlier time-point during immunotherapy or immunogenic therapy, is indicative of a positive response of the immunotherapy or the immunogenic therapy.
5. The method according to paragraph 4 wherein the expression level of at least 4 retrotransposons is determined and wherein a decrease in expression level of at least 1 of the at least 4 retrotransposons is detected relative to the expression levels of the same retrotransposons in a sample obtained from the subject prior to immunotherapy or immunogenic therapy or in a sample obtained at an earlier time-point during immunotherapy or immunogenic therapy, wherein said decrease in expression level of the at least 1 retrotransposon is indicative of a positive response of the immunotherapy or the immunogenic therapy.
6. A method of determining prior to or early after start of immunotherapy or of an immunogenic therapy the outcome of the immunotherapy or the immunogenic therapy, or of determining susceptibility to the immunotherapy or the immunogenic therapy of a tumor in a subject, comprising the step of detecting the expression level of at least 4 retrotransposons in a sample obtained from the subject, and wherein the retrotransposons are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; wherein an increased expression level of at least 1 of the at least 4 retrotransposons is detected relative to the expression level of the same retrotransposons in a control sample or compared to a standard value, and wherein the increased expression level of the at least 1 retrotransposon is indicative of a positive outcome of the immunotherapy or the immunogenic therapy or is indicative of susceptibility of the tumor to the immunotherapy or the immunogenic therapy.
7. A method of determining response to immunotherapy or to immunogenic therapy of a tumor in a subject, comprising the step of detecting the expression level of at least 4 retrotransposons in a sample obtained from the subject, and wherein the retrotransposons are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein a decrease in expression level of at least 1 of the at least 4 retrotransposons is detected relative to the expression levels of the same retrotransposons in a sample obtained from the subject prior to immunotherapy or immunogenic therapy or in a sample obtained at an earlier time-point during immunotherapy or immunogenic therapy, wherein said decrease in expression level of the at least 1 retrotransposon is indicative of a positive response of the immunotherapy or the immunogenic therapy.
8. The method according to any of paragraphs 1, 2 or 4 wherein the at least one retrotransposon is selected from the retrotransposons HERV9-int/AluY (chr12), THE1D (chr4), or MIRb (chrX), as defined in Table 3 or Table 5.
9. The method according to any of paragraphs 1 to 8 wherein the retrotransposons are further selected from the retrotransposons HERVE_a-int (chrY), HERVK14C-int (chrY), HERV17-int (chrY), and L1ME2 (chrY), wherein all retrotransposons are defined in Table 3.
10. The method according to paragraph 3, 5, 6 or 7 wherein one of the at least 4 retrotransposons is selected from the retrotransposons HERV9-int/AluY (chr12), THE1D (chr4), or MIRb (chrX), as defined in Table 3 or Table 5.
11. The method according to any of paragraphs 1 to 10 further including detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES).
12. The method according to paragraph 11 wherein the markers of tumor mutational burden are chosen from substitution markers, indel markers, and microsatellite instability markers.
13. The method according to any of paragraphs 1 to 12 wherein the tumor is melanoma.
14. An immunotherapeutic or immunogenic agent for use in treating a tumor, for use in inhibiting tumor progression or tumor relapse, or for use in inhibiting tumor metastasis, comprising:
   detecting an increased expression level of at least one retrotransposon in a sample obtained from the subject having the tumor, wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein the increased expression level is relative to the expression level of the same retrotransposon in a control sample or compared to a standard value;
   administering a therapeutically effective amount of the immunotherapeutic or immunogenic agent to the subject if an increased expression level of at least one retrotransposon is detected.
15. An immunotherapeutic or immunogenic agent for use in treating a tumor, for use in inhibiting tumor progression or tumor relapse, or for use in inhibiting tumor metastasis, comprising:
   detecting the expression level of at least one retrotransposon in a sample obtained from the subject having the tumor, wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5;
   detecting an increased expression level of at least one retrotransposon in the sample compared to the expression level of the same retrotransposon in a control sample or compared to a standard value;
   administering a therapeutically effective amount of the immunotherapeutic or immunogenic agent to the subject if an increased expression level of at least one retrotransposon is detected.
16. The immunotherapeutic or immunogenic agent for use according to paragraph 14 or 15 wherein the expression level of at least 4 retrotransposons is determined and wherein an increase in expression level of at least 1 of the at least 4 retrotransposons is detected relative to the expression level of the same retrotransposons in a control sample or compared to a standard value.
17. An immunotherapeutic or immunogenic agent for use in treating a tumor, for use in inhibiting tumor progression or tumor relapse, or for use in inhibiting tumor metastasis, comprising:
   detecting the expression level of at least 4 retrotransposons in a sample obtained from the subject having the tumor, wherein the retrotransposons are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein wherein an increase in expression level of at least 1 of the at least 4 retrotransposons is detected relative to the expression level of the same retrotransposons in a control sample or compared to a standard value;
   administering a therapeutically effective amount of the immunotherapeutic or immunogenic agent to the subject if an increased expression level of the at least 1 retrotransposon is detected.
18. An immunotherapeutic or immunogenic agent for use in treating a tumor, for use in inhibiting tumor progression or tumor relapse, or for use in inhibiting tumor metastasis, comprising:
   detecting the expression level of at least 4 retrotransposons in a sample obtained from the subject having the tumor, wherein the retrotransposons are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5;
   detecting an increased expression level of at least 1 of the at least 4 retrotransposons in the sample compared to the expression level of the same retrotransposon in a control sample or compared to a standard value;
   administering a therapeutically effective amount of the immunotherapeutic or immunogenic agent to the subject if an increased expression level of the at least one retrotransposon is detected.
19. An immunotherapeutic or immunogenic agent for use according to any one of paragraphs 14 to 18 wherein the retrotransposons are further selected from the retrotransposons HERVE_a-int (chrY), HERVK14C-int (chrY), HERV17-int (chrY), and L1ME2 (chrY), wherein all retrotransposons are defined in Table 3.
20. An immunotherapeutic or immunogenic agent for use according to paragraph 14 or 18 wherein the at least one retrotransposons is selected from the retrotransposons HERV9-int/AluY (chr12), THE1D (chr4), or MIRb (chrX), as defined in Table 3 or Table 5.
21. An immunotherapeutic or immunogenic agent for use according to paragraph 16 to 18 wherein one of the at least 4 retrotransposons is selected from the retrotransposons HERV9-int/AluY (chr12), THE1D (chr4), or MIRb (chrX), as defined in Table 3 or Table 5.
22. The immunotherapeutic or immunogenic agent for use according to any of paragraphs 14 to 21 wherein the tumor is melanoma.
23. Use of a panel of retrotransposons in a method according to any of paragraphs 1 to 13, wherein the panel is comprising 2 to 62 retrotransposons selected from Table 3 or Table 5.
24. A panel of retrotransposons for use in a method according to any of paragraphs 1 to 13, wherein the panel is comprising 2 to 62 retrotransposons selected from Table 3 or Table 5.
25. A kit for use in a method according to any of paragraphs 1 to 13, wherein the kit is comprising the tools to detect the expression level of at least one retrotransposon selected from Table 3 or Table 5.
26. The kit according to paragraph 25 which is comprising the tools to detect the expression level of 62 retrotransposons selected from Table 3 or Table 5.
27. The kit according to paragraph 25 or 26 further including the tools for detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES).
28. The kit according to any of paragraphs 25 to 27 which is including the tools for detecting the status of at most 500 markers.
29. The method according to any one of paragraphs 1 to 13 wherein at least one analysis step is performed by a computer system or via a computer program product.
30. A computer product comprising a computer readable medium storing instructions for operating a computer system to perform at least one analysis step of a method according to any one of paragraphs 1 to 13.

### EXAMPLES

### 1. Methods

### 1.1. Materials

All materials were molecular biology grade. Unless noted otherwise, all were from Sigma (Diegem, Belgium).

### 1.2. Cell Lines

MCF7, RCC4, SK-MEL-28, A549, 4T1, MC38 and CT26 cell lines were obtained from the American Type Culture Collection and their identity was not further authenticated. These are not listed in the database of commonly misidentified cell lines maintained by ICLAC. MCF7 HIF1B-knockout cells were previously described (Ahmed et al. 2013, Toxicol Sci 138:89-103). MCF7, RCC4, A549, MC38 and 4T1 cells were cultured at 37° C. in Dulbecco's modified Eagle medium (DMEM) with 10% fetal bovine serum (FBS), 5 mL of 100 U/mL Penicillin-Streptomycin (Pen Strep, Life Technologies) and 5 mL of L-Glutamine 200 mM. SK-MEL-28 and CT26 cell lines were cultured at 37° C. in Roswell Park Memorial Institute 1640 Medium (RPMI) with 10% FBS 1% Penicillin-Streptomycin and 1% L-Glutamine.

Murine embryonic stem cells (mESCs) that were triple-knockout for Dnmt1, Dnmt3a and Dnmt3b (Dnmt-TKO), triple-knockout for Tet1, Tet2 and Tet3 (Tet-TKO) and their appropriate wild-type (WT) control mESCs were obtained from Dr. Masaki Okano and Dr. Guoliang Xu, respectively (Hu et al. 2014, Cell Stem Cell 14:512-522; Sakaue et al. 2010, Curr Biol 20:1452-1457). mESCs that were knockout (KO) for Hif1b and their WT control mESCs were previously described (Maltepe et al. 1997, Nature 386:403). Murine Dnmt-WT, Tet-TKO, Tet-WT, Hif1b-WT and Hif1b-KO ESCs were cultured feeder-free in fibroblast-conditioned medium (DMEM with 4,500 mg/L glucose, 2 mM L-glutamine, 1 mM sodium pyruvate, 15% FBS, 1% Penicillin-Streptomycin, 0.1 mM of non-essential amino acids, 0.1 mM β-mercaptoethanol) on 0.1% gelatin coated plates. mESCs from the 159 background (murine ES 159 cells) used for the recombinase-mediated cassette exchange reaction were provided by Prof. Dirk Schubeler (Friedrich Miescher Institute for Biomedical Research, Basel, Switserland) and grown in ESC medium (DMEM with 4,500 mg/L glucose, 2 mM L-glutamine, 1 mM sodium pyruvate, 15% FBS, 1% Pen Strep, 0.1 mM of non-essential amino acids, 0.1 mM β-mercaptoethanol, 10³ U LIF ESGRO (Millipore)) containing 25 μg/mL hygromycin (50 μl of 5 mg/mL stock per 10 mL medium) for at least 10 days.

All cell cultures were confirmed to be Mycoplasma-free every month.

1.3. Cell Line Treatment Conditions

Cell cultures were grown under atmospheric oxygen concentrations in the presence of 5% CO₂, or rendered hypoxic by incubating them under 0.5% oxygen (5% CO₂ and 94.5% N₂). For chromatin-immunoprecipitation coupled to high-throughput (ChIP-seq) experiments, hypoxia was induced during 16 hours, whereas 24 hours of exposure were used when assessing effects of hypoxia on gene or protein expression level. Where indicated, cells were pre-treated with 5-aza-2′-deoxycytidine (aza, 1 μM) for 3 days by adding the correct volume to fresh culture medium while using an equal volume of the carrier (DMSO) as control. This was followed by another day of exposure to aza in hypoxia or normoxia, bringing the total aza exposure time for experiments to 4 days. 2 mM of DMOG (dimethyloxalylglycine, Sigma) was added to culture medium for 24 hours where indicated. Cells were always plated at a density tailored to reach 80-95% confluence at the end of the treatment. Fresh medium was added to the cells just prior to hypoxia. To prove that the extent to which cells were exposed to hypoxia was similar across experiments, we assessed that induction of hypoxia marker genes (BNIP3, EGLN3, CA9, ALDOA) but not HIF1A occurred in each experiment. For experiments involving exposure to aza, we assessed the expression of cancer testis antigens as a positive control.

1.4. DNA Extraction

After exposure to the aforementioned stimuli, cultured cells were washed on ice with ice-cold phosphate-buffer saline (PBS), detached using cell scrapers and collected by centrifugation (400×G, 4° C.). Nucleic acids were subsequently extracted using the Wizard Genomic DNA Purification (Promega, Leiden, The Netherlands) kit according to instructions, dissolved in 200 μL PBS with RNAse A (200 units, NEB, Ipswich, Mass., USA), incubated for 10 minutes at 37° C. After proteinase K addition (200 units) and incubation for 30 minutes at 56° C., DNA was purified using the QIAQuick blood and tissue kit, eluted in 100 μL of a 10 mM Tris, 1 mM EDTA solution (pH 8) and stored at −80° C. until further processing.

1.5. LC-ESI-MS/MS of DNA to Measure 5mC

DNA was extracted and processed for LC-ESI-MS/MS to determine 5mC concentrations exactly as described previously (Thienpont et al. 2016, Nature 537:63-68). To measure the cytosine and 5-methylcytosine content of DNA samples, three technical replicates were run for each sample. More specifically, 0.5 to 2 μg DNA in 25 μL H₂O were digested as follows: an aqueous solution (7.5 μL) of 480 μM ZnSO₄, containing 42 units Nuclease 51, 5 units antarctic phosphatase, and specific amounts of labeled internal standards were added and the mixture was incubated at 37° C. for 3 h in a Thermomixer comfort (Eppendorf). After addition of 7.5 μL of a 520 μM [Na]2-EDTA solution containing 0.2 units snake venom phosphodiesterase I, the sample was incubated for another 3 h at 37° C. The total volume was 40 μL. The sample was then kept at −20° C. until the day of analysis. Samples were then filtered by using an AcroPrep Advance 96 filter plate 0.2 e ISupor (Pall Life Sciences) and then analyzed by LC-ESI-MS/MS. LC-ESI-MS/MS analysis was performed using Ultimate 3000 UPLC (Thermo Scientific, Bremen, Germany) equipped with an Acquity UHPLC HSS T3 column (100×2.1 mm, 1.8 μm particle size) in line connected to a Q-Exactive mass spectrometer (Thermo Fisher Scientific). DNA samples were digested to give a nucleoside mixture and spiked with specific amounts of the corresponding isotopically labeled standards before LC-MS/MS analysis.

A linear gradient was carried out using solvent A (0.05% formic acid) and solvent B (0.05% formic acid, acetonitrile). Practically, samples were loaded at 0.5% solvent B and from 2 to 10 min a ramp to 80% solvent B was carried out and maintained until 12 min. From 12 min to 12.1 min the gradient returned to 0.5% B and this was maintained until 15 min. The flow rate was kept constant at 250 uL/min and the column temperature was set at 40° C. throughout the run.

The mass spectrometer operated in targeted MS2. Normalised collision energy was set to 10. The mass spectrometer ran in positive polarity, the source voltage was 5.0 kV, and the capillary temperature was set at 350° C. Additional sheat gas flow was put at 60 and auxiliary gas flow rate at 10. Auxiliary gas heater temperature was put at 350° C. AGC target was set at 5e4 ions with a maximum ion injection time of 100 ms acquired at a resolution of 17.500. For the data analyses, the peak areas were integrated using the Thermo XCalibur Quan Browser software (Thermo Scientific).

1.5.1. Determination and Comparison of Nucleoside Concentrations

The resulting cytosine and 5-methylcytosine peak areas were normalized using the isotopically labeled standards, and expressed relative to the total cytosine content (i.e. C+5mC). Concentrations were depicted as averages of independent biological replicates, and compared between control and treated conditions, using a paired Student's t-test. No statistical methods were used to predetermine sample size.

1.6. Hypoxia Marker Gene Induction

RNA Extraction, cDNA Synthesis and qPCR

For RNA extractions, cell culture medium was removed, TRIzol (Life Technologies) added and processed according to manufacturer's guidelines. Reverse transcription and qPCR were performed using 2×TaqMan® Fast Universal PCR Master Mix (Life Technologies), TaqMan probes and primers (IDT, Leuven, Belgium) whose sequence is available upon request. Thermal cycling and fluorescence detection were done using a LightCycler 480 Real-Time PCR System (Roche). Taqman assay amplification efficiencies were verified using serial cDNA dilutions, and estimated to be >95%.

We analyzed mRNA levels of genes encoding the E1B 19K/Bcl-2-binding protein Nip3 (BNIP3) and fructose-bisphosphate aldolase (ALDOA), 2 established hypoxia marker genes (Sermeus et al. 2008, Mol Cancer 7:27). Hypoxia markers expression was normalized by using the average of two endogenous controls whose sequence is available upon request. It was moreover excluded that the increase in HIF1a protein concentrations was secondary to a transcriptional upregulation, by assessing HIF1A mRNA expression in parallel. mRNA concentrations were expressed relative to normoxic controls. Differences in mRNA concentration were assessed using a Student's t-test on at least 3 independent biological replicates.

1.7. Western Blot

To assess HIF1a protein stabilization, proteins were extracted from cultured cells as follows: cells were placed on ice, washed twice with ice-cold PBS and lysed in protein extraction buffer (50 mM Tris HCl, 150 mM NaCl, 1% Triton X-100, 0.5% Na-deoxycholate, 0.1% SDS and 1× protease inhibitor cocktail (Roche)). Protein concentrations were determined using a bicinchoninic acid protein assay (BCA, Thermo Scientific) following the manufacturer's protocol. An estimated 60 μg of protein was loaded per well on a NuPAGE Novex 3-8% Tris-Acetate Protein gel (Life Technologies), separated by electrophoresis and blotted on polyvinylidene fluoride membranes. Membranes were activated with methanol and washed with Tris-buffered saline (TBS; 50 mM Tris HCl, 150 mM NaCl) with 0.1% Tween 20, and incubated with rabbit α-tubulin (2144S, Cell Signaling), rabbit β-actin (4967, Cell Signaling), rabbit HIF-1β/ARNT (D28F3) XP® (5537, Cell Signaling) at 1:1,000 dilution and rabbit HIF-1α (C-Term) Polyclonal Antibody (Cayman Chemical Item 10006421) 1:3,000. Incubation with the secondary antibodies and detection were performed according to routine laboratory practices. Western blotting was done on 3 independent biological replicates.

1.8. Analysis of HIF1β Target Genes Using ChIP-Seq

20-25×10\hat{ }6 cells were incubated in hypoxic conditions for 16 hours. Cultured cells were subsequently immediately fixed by adding 1% formaldehyde (16% formaldehyde (w/v), methanol-free, Thermo Scientific) directly in the medium and incubating for 8 min on a flat-bed shaker at room temperature. Fixed cells were incubated with 150 mM of glycine for 5 min to revert the cross-links, washed twice with ice-cold PBS 0.5% Triton-X100, scraped and collected by centrifugation (1000 g, 5 min at 4° C.). The pellet was resuspended in 1,400 μL of RIPA buffer (50 mM Tris-HCl pH 8, 150 mM NaCl, 2 mM EDTA pH 8, 1% Triton-X100, 0.5% sodium deoxycholate, 1% SDS, 1% protease inhibitors) and transferred to a new Eppendorf tube. The lysate was homogenized by passing through an insulin syringe, and incubated on ice for 10 min. The chromatin was sonicated for 3 min by using a Branson 250 Digital Sonifier with 0.7 sec ‘On’ and 1.3 sec ‘Off’ pulses at 40% power amplitude, yielding predominantly fragment sizes between 100 and 500 bp. The sample was kept ice-cold at all times during the sonication. Next, samples were centrifuged (10 min at 16,000 g at 4° C.) and supernatant transferred in a new Eppendorf tube. Protein concentration was assessed using a BCA assay. Fifty μL of sheared chromatin was used as “input” and 1.4 μg of primary ARNT/HIF1β monoclonal antibody (NB100C124, Novus) per 1 mg of protein was added to the remainder of the chromatin and incubated overnight at 4° C. in a rotator. Next, Pierce Protein A/G Magnetic Beads (Life Technologies) were added to the samples in a volume that is 4× the volume of the primary antibody and incubated at 4° C. for at least 5 hours. A/G Magnetic Beads were collected and washed 5 times with washing buffer (50 mM Tris-HCl, 200 mM LiCl, 2 mM EDTA, pH 8, 1% Triton, 0.5% Sodium deoxycholate, 0.1% SDS, 1% protease inhibitors), and twice with TE buffer. The A/G magnetic beads were resuspended in 50 μL of TE buffer, and 1.5 μL of RNAse A (200 units, NEB, Ipswich, Mass., USA) were added to the A/G beads samples and to the input, incubated for 30 min at 37° C. After addition of 1.5 μL of Proteinase K (200 units, NEB) and overnight incubation at 65° C. on a stirrer, the beads were removed from the solution using a magnet and DNA was purified using 1.8× volume of Agencourt AMPure XP (Beckman Coulter) according to the manufacturer's instructions. DNA was eluted in 20 μL of TE buffer. The input DNA was quantified on NanoDrop. Next, 1 μg of the input and all the immunoprecipitated DNA was converted into sequencing libraries using the NEBNext DNA library prep master mix set (NEB) following manufacturer's instructions.

A single end of these libraries was sequenced for 50 bases on a HiSeq 2000, mapped using Bowtie and extended for the average insert size (250 bases). ChIP peaks were called by Model-based Analysis for ChIP-Seq (MACS)(Feng et al. 2011, Curr Protoc Bioinformatics Chapter 2, Unit 2 14), with standard settings and using read counts from an input sample as baseline.

HIF1β binding peak positions in the human cell lines MCF7 (both vehicle- and aza-treated), RCC4, A549 and SK-MEL-28 were defined by using the stringent threshold P<10\hat{ }-15. A threshold equal to P<10\hat{ }-5 was used to define HIF1β binding peaks in murine Dnmt-WT and Dnmt-TKO ESCs.

To compare HIF1β binding peaks between human cell lines (MCF7, RCC4, A549 and SK-MEL-28), HIF1β binding peaks were called as present if the average coverage at the 200 bps centered on the summit was >4-fold bigger than the local background, and as absent if it was <2.5-fold smaller than the local background, with local background being defined as the read depth across regions 1.5-5 kb up- and downstream of the peak. Intermediate coverage was annotated as unclassified. To compare HIF1β binding peaks between murine Dnmt-WT and -TKO ESCs, HIF1β binding peak was called as present if the average coverage at the 200 bp centered on the summit was >4-fold bigger than the background, and as absent if it was <4-fold smaller than the background.

To compare efficiency between experiments, scatter plots of reads counts at peak regions of HIF1β binding regions were generated per cell line in a pairwise fashion. Each pairwise correlation was characterized by a high Pearson coefficient (R0.65 for MCF7 cells, R0.63 for MCF7 aza-exposed cells, R0.63 for RCC4 cells, and R=0.44, 0.92 and 0.65 for SK-MEL cells). Pairwise correlation coefficients for murine Dnmt-WT ESCs (n=4 replicates) and murine Dnmt-TKO ESCs (n=3 replicates) were similarly strongly correlated (Dnmt-WT: R0.67 and Dnmt-TKO ESCs: R0.95).

1.9. Annotation of Genomic Features

Human sequences were mapped to genome build hg19 and murine sequences to genome build mm10. Putative HIF binding sites were detected genome-wide by screening the whole genome for RCGTG motifs using the regular expression search tool dreg (www.bioinformatics.nl/cgi-bin/emboss/help/dreg). The frequency per bp of RCGTG motifs inside HIF1β binding peaks and in the rest of the genome was calculated, and enrichment of RCGTG motifs at HIF1β binding peaks quantified by overlapping RCGTG positions in the genome with the HIF1β binding peak positions as defined by MACS.

The distances of HIF1β peaks to the nearest RCGTG motif (cumulative frequency), transcription start site and open chromatin (frequency) were calculated by overlapping each genomic feature with HIF1β peak positions using BedTools in R (Alexa & Rahnenfuhrer 2010, R Package version 2.12.0). Protein-coding genes were annotated as per in Ensembl version 92. Promoter regions were annotated as being 2 kb upstream or 500 bp downstream of the start site of each gene. Chromatin state annotation of MCF7 and murine ESCs was as described (Taberlay et al. 2014, Genome Res 24:1421-1432; Bogu et al. 2015, Mol Cell Biol 36:809-819). HIF1β binding peaks were annotated with these features and overlapped with the repeat genome using BedTools.

1.10. Genome Distribution of 5mC: BS-Seq, SeqCapEpi BS-Seq and mDIP-Seq

5mC DNA IP-seq (mDIP-seq), bisulfite sequencing (BS-seq) and SeqCapEpi BS-seq were applied exactly as described previously (Thienpont et al. 2016, Nature 537:63-68). To quantify DNA methylation inside HIF1β binding peaks, SeqCapEpi BS-seq probes with >40× coverage were overlapped with HIF1β binding peaks as defined by MACS. Methylation levels at the probes overlapping and non-overlapping (rest of the genome) HIF1β binding peaks were calculated using Seqmonk.

ChIP-Bisulfite-sequencing (ChIP-BS-seq) was done as ChIP-seq, except that methylated adaptors (NEB) were ligated, and DNA libraries were bisulfite converted using the EZ DNA Methylation-Lightning™ kit (Zymo) prior to library amplification using HiFi Uracil+(KAPA). Libraries were mapped using Bismark as described (Thienpont et al. 2016, Nature 537:63-68).

1.10.1. BS-Seq and SeqCapEpi BS-Seq

To confirm the unmethylated status of the DNA bound by HIF using an independent method, DNA libraries were prepared using methylated adapters and the NEBNext DNA library prep master mix set following manufacturer recommendations. Libraries were bisulfite-converted using the Imprint DNA modification kit (Sigma) as recommended, and PCR amplified for 12 cycles using barcoded primers (NEB) and the KAPA HiFi HS Uracil+ ready mix (Sopachem, Eke, Belgium) according to manufacturer's instructions. Fragments were selected from these libraries using the SeqCap Epi CpGiant Enrichment Kit (Roche) following the manufacturer's instructions, sequenced from both ends for 100 bases on a HiSeq 2000.

For analyzing these sequences, sequencing reads were trimmed for adapters using TrimGalore and mapped on a bisulfite-converted human genome (GRCh37) using BisMark.

1.10.2. Quantification of DNA Methylation Inside HIF16 Binding Peaks

SeqCapEpi probes with >40× coverage were overlapped with HIF1β binding peaks as defined by MACS. Methylation level at the probes overlapping and not overlapping (rest of the genome) HIF1β binding peaks were calculated using Seqmonk.

1.10.3. DIP-Seq

Library preparations and DNA immunoprecipitations were as described (Taiwo et al. 2012, Nature Protocols 7:617-636), using established antibody targeting 5mC (clone 33D3, Eurogentec, Liege, Belgium). A single end of these libraries was sequenced for 50 bases on a HiSeq 2000, mapped using Bowtie and extended for the average insert size (150 bases).

1.10.4. ChIP-Bisulfite-Sequencing (ChIP-BS-Seq)

ChIP-BS-seq was done as ChIP-seq, except that methylated adaptors (NEB) were ligated, and DNA libraries were bisulfite converted using the EZ DNA Methylation-Lightning™ kit (Zymo) prior to library amplification using HiFi Uracil+(KAPA). Libraries were mapped as described for BS-seq.

1.11. RNA-Seq

To assess the impact of the HIF binding at gene promoters on their expression, strand-specific RNA-seq was performed in human cell lines and murine Dnmt-WT and Dnmt-TKO ESCs. Briefly, total RNA was extracted using TRIzol (Invitrogen), and remaining DNA contaminants in 17-20 μg of RNA were removed using Turbo DNase (Ambion) according to the manufacturer's instructions. RNA was repurified using the RNeasy Mini Kit (Qiagen). For total RNA-seq, ribosomal RNA present was depleted from 5 μg of total RNA using the RiboMinus Eukaryote System (Life technologies). cDNA synthesis was performed using the SuperScriptR III Reverse Transcriptase kit (Invitrogen). 3 μg of random Primers (Invitrogen), 8 μL of 5× First-Strand Buffer and 10 μL of RNA mix were incubated at 94° C. for 3 min and then at 4° C. for 1 min. Next, 2 μL of 10 mM dNTP Mix (Invitrogen), 4 μL of 0.1 M DTT, 2 μL of SUPERase⋅ In RNase Inhibitor 20U/μL (Ambion), 2 μL of SuperScript III RT (200 units/4) and 8 μL of Actinomycin D (1 μg/μL) were added and the mix was incubated 5 min at 25° C., 60 min at 50° C. and 15 min at 70° C. to heat inactivate the reaction. The cDNA was purified using 80 μL (2× volume) of Agencourt AMPure XP and eluted in 50 μL of the following mix: 5 μL of 10×NEBuffer 2, 1.5 μL of 10 mM dNTP mix (10 mM dATP, dCTP, dGTP, dUTP, Sigma), 0.1 μL of RNaseH (10 U/4, Ambion), 2.5 μL of DNA Polymerase I Klenow (10 U/4, NEB) and water until 50 μL. The eluted cDNA was incubated for 30 min at 16° C., purified by Agencourt AMPure XP and eluted in 30 μL of dA-Tailing mix (2 μL of Klenow Fragment, 3 μL of 10×NEBNext dA-Tailing Reaction Buffer and 25 μL of water). After 30 min incubation at 37° C., the DNA was purified by Agencourt AMPure XP, eluted in TE buffer and quantified on NanoDrop. Subsequent library preparation was done using the DNA library prep master mix set and sequencing was performed as described for ChIP-seq.

mRNA capture and stranded library preparation of RNA from MCF7 cells, mouse cell lines and tumours for the purpose of retrotransposon expression analysis was performed using the KAPA Stranded mRNA-Seq Kit (Illumina) according to the provided protocol.

1.12. RNA-Seq Analyses

RNA-seq data were expressed in transcript per million (TPM), 0.01 offset. Expression read counts of retrotransposons are calculated using the RepEnrich tool (https://github.com/nerettilab/RepEnrich), and normalized to the total mappable read depth. The repeat genome of the human reference genome hg19 was download from the RepEnrich website. Human retrotransposon classes (LINE, SINE, LTR) contain 16 families and 779 subfamilies. The repeat genome of the mouse genome mm10 was built using the repeat masker track from the UCSC genome browser. Mouse retrotransposon classes (LINE, SINE, LTR) contain 24 families and 906 subfamilies.

Differential gene expression was quantified by EdgeR, normalizing to the sum of the mapped expression counts.

Expression of cancer testis antigens was annotated according to all entries listed in the CTDatabase (www.cta.lncc.br/modelo.php).

1.13. Ribo-seq Ribo-seq data (Bai et al. 2016, Nat Commun 7:12310) were mapped to the reference genome (build hg19) and to the corresponding repeat genome. Only expressed genes (>1 read per million) were retained, and the ratio of polysome:monosome was calculated (Gao et al. 2015, Nat Methods 12:147-153).

1.14. Gene Ontology Analysis

Genes were associated to ontologies as annotated in BioMART (Ensembl GRCh37 release 84), and enrichment of ontologies was analysed using TopGo version 1.0 in R (Alexa & Rahnenfuhrer 2010, R Package version 2.12.0), using the classic algorithm, contrasting to all protein-coding genes.

1.15. Structural Modelling of DNA Methylation

The crystal structure of HIF2α:HIF1β in complex with DNA containing the RCGTG core sequence 5′-ACGTG-3′ (Wu et al. 2015, Nature 524: 303-308; PDB code 4ZPK) was used as a template for introducing and analyzing the structural consequences of methyl groups at position 5 of the cytosines using the programs PyMOL (Schrodinger, LLC) and Chimera (Pettersen et al. 2004, J Comput Chem 25:1605-1612).

1.16. Microscale Thermophoresis (MST) Binding Assay

MST measurements were performed in triplicate using the NanoTemper Monolith NT.115 instrument. The two protein complexes (HIF1α-HIF1β and HIF2α-HIF1β) were purified as described earlier (Wu et al. 2015, Nature 524:303-308). They were both labeled using Monolith NT Protein labeling kit RED NHS (Nano Temper technologies). Oligonucleotides were from IDT. In brief, 25 nM of each labeled protein were mixed in 16 serial dilutions of 1:1 with different DNA concentrations starting from a concentration of 25 μM. The experiment was carried out in 20 mM phosphate buffer, 75 mM NaCl, 5 mM DTT, 0.05% Tween-20, pH 7.4. Samples were incubated for 20 min on ice prior to loading 5 μL of each sample into the standard treated capillaries. MST measurements were carried out at 25° C. at 20% LED power and medium MST power. Data was normalized to % fraction bound and the values for the equilibrium dissociation constant (K_{D}) were calculated by fitting the curves in GraphPad Prism 7.

1.17. Generation of Murine ES Cells Containing a Methylated or Unmethylated Human HIF Binding Region

The DNA fragment (chr16:30,065,212-30,065,711) containing five CGTG motives was selected based on high HIF1β ChIP-enrichment in MCF7, RCC4 and SK-MEL-28 cells. Oligonucleotides were designed to amplify the target region (AGGTGCAATTGTTCCTCCGCCTCCCTTAC (SEQ ID NO:1) and AAGGGCAATTGCCGAGC 11111 CCTTTACGA (SEQ ID NO:2)), and used for PCR amplification of the target region using the Q5R Hot Start High-Fidelity 2× Master Mix (NEB), followed by evaluation of the PCR products by gel electrophoresis and purification with the Qiaquick PCR purification kit (28104, Qiagen). These PCR primers were evaluated for specificity in human (MCF7, RCC4, SK-MEL-28) but not in the mouse genomic DNA, and MfeI restriction sites were added to the ends of the primer pairs. The purified amplicon was digested with the appropriate enzyme and cloned into the L1-poly-L1 plasmid (provided by Prof. Dirk Schubeler, Friedrich Miescher Institute for Biomedical Research, Basel, Switzerland), containing a multiple cloning site flanked by two inverted L1 Lox sites. Correct insertion and sequence identity were verified by Sanger sequencing. This plasmid was in vitro methylated using M.Sssl (NEB) according to the manufacturer's instructions, and purified using isopropanol precipitation. Successful and complete in vitro methylation was confirmed by bisulfite-conversion (EZ DNA Methylation-Lightning Kit, D5031, Laborimpex), PCR amplification using the MegaMix Gold 2× Mastermix (Microzone) and Sanger sequencing. 10 μg of pIC-CRE plasmid and 25 μg of (un)methylated plasmid were electroporated in mouse ES 159 cells containing an L1-flanked thymidine kinase expression cassette (provided by Prof. Dirk Schubeler, Friedrich Miescher Institute for Biomedical Research, Basel, Switzerland). After electroporation, cells were plated and maintained in nonselective ES medium for 1 day, and from the second day onwards cultured in ES medium containing 10 μM ganciclovir. After 10 to 12 days, individual clones of the surviving cells were picked and transferred to ESC medium in 96-well plates, were then gradually expanded and, following DNA extraction, assessed for occurrence of successful insertion events using PCR (using the oligonucleotides defined in SEQ ID Nos:1 and 2) and gel electrophoresis.

To verify maintenance of the methylation levels of the cloned HIF binding site, genomic DNA was extracted from a positive clone. 500 ng of DNA was bisulfite-converted using the EZ DNA Methylation-Lightning Kit (D5031, Laborimpex) and amplified using the MegaMix Gold 2× mastermix and validated primer pairs for the target locus (Forward: GTTTGGGTTAGTGATAGGGTGT (SEQ ID NO:3), Reverse: AAACCCTCCCTTCTACTCCTTTCC (SEQ ID NO:4)). Per sample, PCR product sizes were verified by gel electrophoresis, and amplicons converted into sequencing libraries using the NEBNext DNA library prep master mix set (E6040L, Bioke). These were next sequenced to a depth exceeding 500×, and mapped and analyzed as described higher.

Positive colonies were expanded into 10 cm dishes and subjected to ChIP as described above. qPCR was performed with the SYBR GreenER qPCR SuperMix Universal (11762500, Life Technologies) on a Quantstudio 12K (Applied Biosystems), by using specific primers for the cloned locus (oligonucleotides TCGTTTCCGACTTTTCCATC (SEQ ID NO:5) and CAGCCAGAATGTTGGCAAT (SEQ ID NO:6)) and an independent murine genomic region for background quantification (oligonucleotides CACTTGCTGAATAATTGGGTGT (SEQ ID NO:7) and CTGTTGTCCAGTTTTCTTCACG (SEQ ID NO:8)). Enrichment was calculated as fold enrichment over background.

1.18. TCGA Samples and Data Analysis

From the TCGA server, we selected 4,494 tumours from 14 cancer types: 407 bladder urothelial carcinoma (BLCA), 689 breast cancer (BRCA), 303 cervical squamous cell carcinoma and endocervical adenocarcinoma (CESC), 237 colon adenocarcinoma (COAD), 100 head and neck squamous cell carcinoma (HNSC), 270 kidney renal papillary cell carcinoma (KIRP), 251 liver hepatocellular carcinoma (LIHC), 442 lung adenocarcinoma (LUAD), 366 lung squamous cell carcinoma (LUSC), 175 pancreatic adenocarcinoma (PAAD), 494 prostate adenocarcinoma (PRAD), 392 skin cutaneous melanoma (SKCM), 193 stomach adenocarcinoma (STAD) and 175 uterine corpus endometrial carcinoma (UCEC) for which RNA data were available. The corresponding RNA-seq read counts were downloaded. DNA methylation data from Infinium HumanMethylation450 BeadChip arrays available were downloaded for the same samples.

To identify which of these tumour samples were hypoxic or normoxic, we performed unsupervised hierarchical clustering based a modification (Ward.D of the clusth function in R's stats package; Alexa & Rahnenfuhrer 2010, R Package version 2.12.0) of the Ward error sum of squares hierarchical clustering method, on normalized log-transformed RNA-seq read counts for 15 genes that make up the hypoxia metagene signature (ALDOA, MIF, TUBB6, P4HA1, SLC2A1, PGAM1, ENO1, LDHA, CDKN3, TPI1, NDRG1, VEGFA, ACOT7, CDKN3 and ADM) (Buffa et al. 2010, Br J Cancer 102:428-435). In each case the top 2 subclusters identified were annotated as normoxic and hypoxic. Retrotransposon expression in TCGA samples was analyzed by applying RepEnrich as described higher, to the associated raw RNA-seq FASTQ files.

1.19. Hif1b-Knockout 4T1 Cells

Four gRNAs targeting two different exons in the Hif1b locus of the mouse genome and one non-targeting gRNA (scramble) were designed with the appropriate restriction sites for the receiver plasmid using the online Crispor tool (http://crispor.tefor.net). Oligonucleotides corresponding to gRNAs were synthesized by IDT, and forward and reverse oligonucleotides were annealed in the CutSmart buffer (B7240S, NEB) before cloning into the LentiGuide-Puro plasmid (Plasmid 52963, Addgene). Positive colonies were screened by PCR and validated by Sanger sequencing. LentiGuide-Puro plasmids containing GFP was used as positive control to evaluate the transfection- and transduction-efficacy.

A transformation mix containing viral particles, TE, CaCl₂, H₂O and LentiGuide-Puro plasmid was added to the HEK 293T cells when reaching 70% confluency. Four plasmids containing the different gRNAs for Hif1b and one plasmid containing the scramble gRNA were used, together with plasmids containing GFP as positive control. Medium was renewed after 14-16 hours and transfection efficiency was evaluated based on GFP expression. After 36 h, supernatant containing the concentrated virus was collected by ultracentrifugation. Virus was dissolved in clean PBS and stored at −150° C.

4T1 cells were transduced with a lentiviral vector expressing a doxycycline inducible Cas9 nuclease (Cat #CAS11229, Dharmacon) for a tight regulation of the Cas9 expression and gene editing. An infection rate of 30% was used to ensure that the majority of transduced cells harbour a single copy of the vector. These 4T1 cells were always kept in selection medium containing 10 μg/mL of blasticidin (ant-bl-05, Invivogen). When reaching 70% confluency, cells were transduced with one titer of virus. After 24h, the virus was removed and transduction efficacy evaluated based on GFP expression. After 48h, puromycin (P9620, Sigma-Aldrich) 1.5 μg/mL medium was added to the medium. Cells were kept in the presence of blasticidin and puromycin for the remaining experimental procedures. After 3-5 days, Cas9 expression was induced by adding doxorubicin (D2975000, Sigma-Aldrich) 0.5 μg/mL medium for 3 days. Cells were kept one day without doxorubicin before injection in the mice or further experimental procedures. 4T1 cells transduced with the four gRNAs targeting Hif1b were expanded and proteins were extracted to test the efficacy of the knockout by western blot. The most efficient gRNA was used to perform the further experiments (F: CACCGTGAAATAGAACGGCGGCGA (SEQ ID NO:9) and R: AAACTCGCCGCCGTTCTATTTCAC (SEQ ID NO:10); Non-targeting: CACCGCACTACCAGAGCTAACTCAG (SEQ ID NO:11) and Non-targeting: AAACCTGAGTTAGCTCTGGTAGTGC (SEQ ID NO:12)). Stability of knockout in 4T1 cells after two weeks was confirmed by western blot.

1.20. Mouse Tumour Model

All the experimental procedures were approved by the Institutional Animal Care and Research Advisory Committee of the KU Leuven. 1×10\hat{ }6 4T1 cells, Hif1b-knockout or wild-type (scramble), were injected orthotopically in the mammary gland of 10 weeks old Balb/c mice, and 1×10\hat{ }6 CT26 or MC38 cells were injected subcutaneously in 10 weeks old Balb/c or C57BL/6J mice, respectively. When the tumour was palpable (starting volume 100 mm³), the mice were injected intraperitoneally with 0.8 mg/kg of 5-aza-2′-deoxycytidine (aza) or PBS, 40 mg/kg DC101 antibody (BE0089, InVivoMab) or IgG (BE0060, InVivoMab) or 10 mg/kg anti-PD1 antibody (6E0146, InVivoMab) or IgG antibody (BE0089, InVivoMab) according to the following schedules: DC101 three times per week; anti-PD1 every other day, starting when the tumour size was around 200 mm³; aza was administered in 2 cycles with 2-days rest in between until the control tumours reached the endpoint. Tumour volumes were monitored every two to three days by a calliper, and mice were culled before tumour volumes exceeded 2,000 mm³. When over 20% of mice were culled, the experiment was terminated (all arms). In vivo experiments in 4T1, CT26 and MC38 treated with aza or anti-PD1 antibody were performed three times, with at least 6 mice per treatment group in each experiment.

1.21. Neo-Epitope Burden

To assess the neo-epitope burden, we mapped RNA sequencing data of isogenic 4T1, B16 and CT26 tumour models, removed duplicate reads from individual samples and merged per tumour model all samples into a single file. In this file, variants were called according to GATK best practices, using GATK3.4. Briefly, reads were split into exon segments and sequences overhanging the non-exonic regions were hard-clipped using split'n'trim. Next, local indel realignment and base recalibration was performed, followed by variant calling with GATK's HaplotypeCaller. After quality filtering for minimal Fisher strand values (30) and minimal read depth (10-fold), we removed SNPs reported in the Sanger Mouse project (rsIDdbSNPv137). Remaining variants were annotated by Annovar (version 2.17.0), and only variants in coding regions were retained. Finally, the neo-epitope burden was expressed as the number of non-SNP variants in coding sequences, normalized to the number of coding sequences that were expressed, the latter being defined as having a minimal read depth of 10.

1.22. Immunofluorescence

Different protocols were applied depending on the epitope of interest: hypoxia (pimonidazole) staining was combined with blood vessel (CD31) staining, as cytotoxic T-cell activity (Granzyme B) and infiltration (CD8a) were combined. General (CD45) and cytotoxic (CD8a) T-cell infiltration were also stained separately. Tumours were harvested, fixed in formaldehyde and embedded in paraffin using standard procedures. Slides were deparafinated and rehydrated in 2 xylene baths (5 min), followed by 5 times 3 min in EtOH baths at decreasing concentrations (100%, 96%, 70%, 50% and water) and a 3 min Tris-buffered saline (TBS; 50 mM Tris, 150 mM NaCl, pH 7.6) bath. Antigen retrieval was done using AgR (DAKO) at 100° C. for 20 min, followed by cooling for 20 min. Slides were washes in TBS for 5 min, endogenous peroxidase activity was quenched using H₂O₂ (0.3% in MeOH), followed by three 5 min washes in TBS. Slides were blocked using pre-immune goat serum (X0907, Dako) or pre-immune rabbit serum (for pimonidazole, X090210, Dako) 20% in TNB. Binding of primary antibodies: FITC-conjugated mouse anti-pimonidazole (HP2-100, Hydroxyprobe), rabbit anti-Gzmb (ab4059, abcam) and rat anti-CD45 (553076, BD Biosciences) all 1:100 in TNB was allowed to proceed overnight. Slides were washed 3 times in TNT (0.5% Triton-X100 in TBS) for 5 min, after which secondary antibodies: peroxidase-conjugated rabbit anti-FITC (PA1-26804, Pierce), Alexa fluor 488-conjugated goat anti-rabbit (A-11034, Thermo Fisher) and biotinylated goat anti-rat (559286, BD biosciences) all 1:100 in TNB with 10% pre-immune goat serum were allowed to bind for 1 hour. Slides were washed 3 times for 5 min in TNT, after which signal amplification was done by 30 min incubation with peroxidase-conjugated streptavidin 1:100 in TNB (for all besides pimonidazole) accompanied by nuclear staining with Hoechst (H3570, Thermo Fisher) 1:500 in TNB only for the single (CD45 or CD8a) stainings, washing (3 times 5 min in TNT) and 8 min incubation using Fluorescein Tyramide (for pimonidazole NEL701A001KT, perkin Elmer) or Cy3 (NEL704A001KT, Perkin Elmer) 1:50 in amplification diluent.

Slides stained for pimonidazole and Gzmb required co-staining for CD31 and CD8a respectively and were subjected to a second indirect staining for the latter epitopes. After 5 min of TNT and 5 min of TBS, slides were quenched again for peroxidase activity using H₂O₂ and blocked using pre-immune goat or rabbit (CD31) serum, prior to a second overnight round of primary antibody binding: rat anti-CD31 (557355, BD Biosciences) or rat anti-CD8a (14-0808-82, Thermo Fisher) 1:100 in TNB. The next day, 3 times 5 min washes with TNT were followed by a 1 hour incubation with biotinylated goat anti-rat (559286, BD biosciences) 1:100 in TNB, again 3 times 5 min washes with TNT, a 30-min incubation with peroxidase-conjugated streptavidine 1:100 in TNB accompanied by nuclear staining with Hoechst (H3570, Thermo Fisher) 1:500 in TNB, 3 times 5 min washes with TNT and signal amplification for 8 min using Cy3 (NEL704A001KT, Perkin Elmer) 1:50 in amplification diluent. Finally, slides were washed 3 times for 5 min with TNT and mounted with Prolong Gold (P36930, Life Technologies).

For immunofluorescence analysis on 4T1 wild-type tumours, slides were imaged on an infraMouse Leica DM5500 microscope. 4 sections from different treatment groups were stained per slide while 6 pictures from different tumour areas were used for processing with Image J. More specifically, nuclei were identified using the Hoechst signal, and signal intensities for Fluorescein (pimonidazole), Alexa fluor 488 (Gzmb) and Cy3 (CD45, CD8a and CD31) were used to detect Gzmb+, CD45+ and/or CD8a+ cells. Analyses were exclusively performed on slide regions showing a regular density and shape of nuclei, in order to avoid inclusion of acellular or necrotic areas. Gzmb+CD8a+ cells were counted directly, allowing the precise quantification of the number of active cytotoxic T cells per tumour. The number of CD45+ cells was used to normalize the number of CD8a+ cells, as such calculating the number of infiltrating cytotoxic T cells compared to the total immune infiltration. CD31-positive regions were quantified manually using Image J. The pimonidazole signal was used together with the Hoechst signal to quantify the percentage of hypoxia per tumour area in each picture and stratify tumours as hypoxic (pimo-high) or normoxic (pimo-low).

For immunohistofluorescence on Hif1b-knockout or -scramble 4T1 grafts, tumours were harvested and snap frozen in liquid nitrogen before temporary storage at −80° C. Thawed tumours were embedded in paraffin and sectioned using standard procedures (5 μm of thickness). In a Leica Autostainer (30 min), slides were deparafinated and rehydrated in 2 xylene baths for 5 min, followed by 5 min in ethanol baths at decreasing concentrations (100%, 96%, 70%, 50% and water). Slides were fixed in 10% neutral buffered formalin for 10 min and rinsed twice in double distilled water. Antigen retrieval proceeded in AR6 buffer (AR600, Perkin Elmer) at 100° C. for 23 min in a pressure cooker, followed by cooling in double distilled water for 20 min. Slides were washes in TBST (TBS with 0.5% Tween 20) for 3 min, and blocked using blocking buffer (pre-immune goat serum (X0907, Dako) 10%, 1% BSA (126575, Millipore) in TBS)) for 30 min. The primary antibody (rabbit anti-Gzmb) 1:1,000 in dilution buffer (1% BSA in TBS) was applied for 30 min at room temperature, followed by 3 washes of 2 min in TBST at room temperature. Slides were next incubated with the secondary antibody (EnVision+/HRP goat anti-rabbit (K4003, Dako)) for 10 min at room temperature, and washed 3 times for 2 min in TBST at room temperature. The OPAL 570 fluorophore (fp1488, PerkinElmer) 10% in amplification diluent (FP1498, PerkinElmer) was applied for 10 min at room temperature followed by 3 washes of 2 min in TBST at room temperature. Slides were stripped by heating in AR6 buffer just below boiling point and cooled down in double distilled water, followed by rinsing in TBST. These steps were repeated starting from blocking for the second staining with primary antibody rat anti-CD8a 1:300, secondary antibody goat anti-rat (MP-7444, Vector) and opal 690 (fp1497, PerkinElmer) and the third staining with rat anti-CD45 1:1,000, secondary antibody goat anti-rat and Opal 520 (fp1487, PerkinElmer). After the third staining, slides were incubated with spectral DAPI (fp1490, PerkinElmer) 10% in TBST for 5 min at room temperature, washed for 2 min in TBST at room temperature and mounted with ProLong Diamond Antifade Mountant (P36961 Invitrogen). Images were acquired on a Zeiss Axio Scan.Z1 using a ×20 objective and ZEN 2 software (Zeiss) with exposure times between 10-50 ms. Image processing was done using QuPath (version 0.1.2). Specifically, following visual inspection of the staining results, cells were first automatically detected using the DAPI channel (cell size constrained between 5 and 400 μm²). Next, a cell classifier was generated using QuPath. Specifically, for 1 slide out of all slides, 5 sets of cells were selected: one set that was positive for CD45, one set that was negative for CD45, and three sets of CD45+ cells positive for CD8, Gzmb and CD8, or Gzmb alone. Using these 5 sets of cells, a random trees classifier was generated. Cell classification was visually verified to have occurred correctly. Next, in each tumour section, a representative region was selected, containing at least 1,000 cells. On these cells, the random trees classifier was subsequently applied. This process was reiterated for all other tumour sections stained for the same set of markers. The resulting cell identities were then exported, and processed in R (Alexa & Rahnenfuhrer 2010, R Package version 2.12.0). For each tumour, average cell frequencies were generated, which were summarized using boxplots.

1.23. Published Data Sets

Published data sets were obtained from GEO under the following accession numbers: HIF1β, HIF1α, HIF2α and isotype ChIP-seq in MCF7: GSM700947, GSM700944, GSM700945, GSM700948 (Schodel et al. 2011, Blood 117:e207-217); WGBS of MCF7: GSM1328112 (Menafra et al. 2014, PLoS One 9:e99603), and Whole-genome bisulfite sequencing (WGBS) of murine WT ESCs: GSM1127953 (Habibi et al. 2013, Cell Stem Cell 13:360-369); CTCF, FOXA1 and GATA3 ChIP-seq in MCF7: GSM1003581, GSM1010727, wgEncodeEH002293; transcription factors in MCF7: GSE41561 (Griffon et al. 2015, Nucleic Acids Res 43:e27); Ribo-seq: GSE81469 (Bai et al. 2016, Nat Commun 7:12310); RNA-seq from the PyMT tumour model: GSE31223, GSE30866 (Hu et al. 2012, Proc Natl Acad Sci USA 109:3184-3189); NOMe-seq: GSE57498 (Taberlay et al. 2014, Genome Res, gr.163485.113).

### EXAMPLES

### 2. Results

### Example 2.1. DNA Methylation of HRE Sites Anti-Correlates with HIF Binding

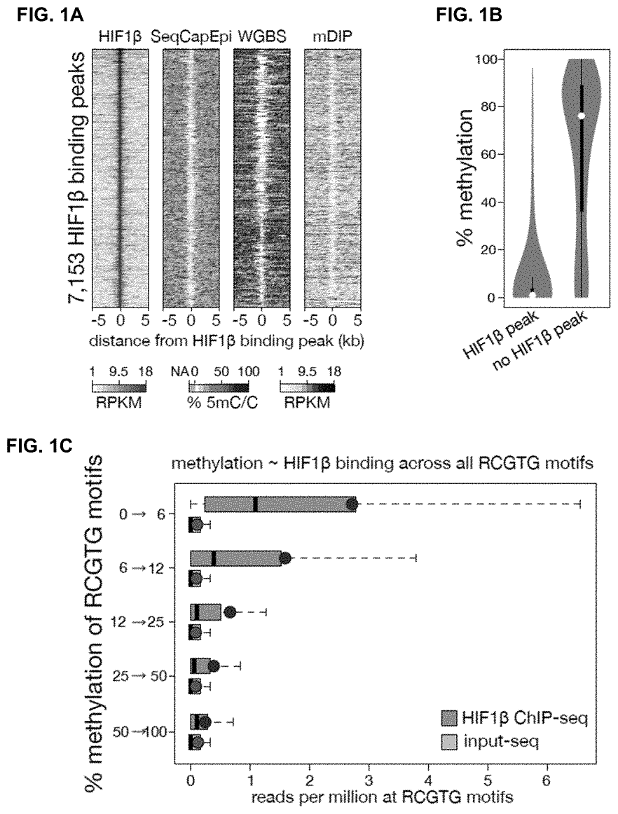

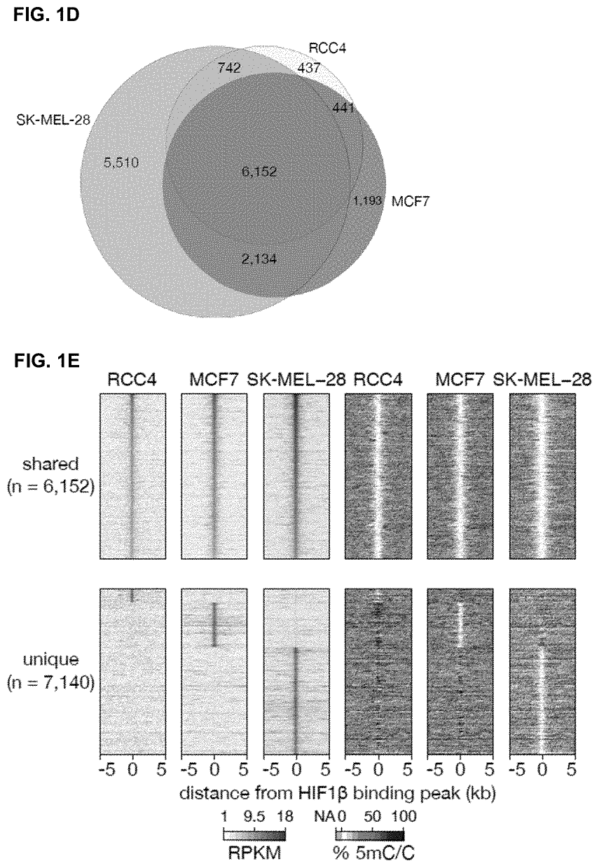

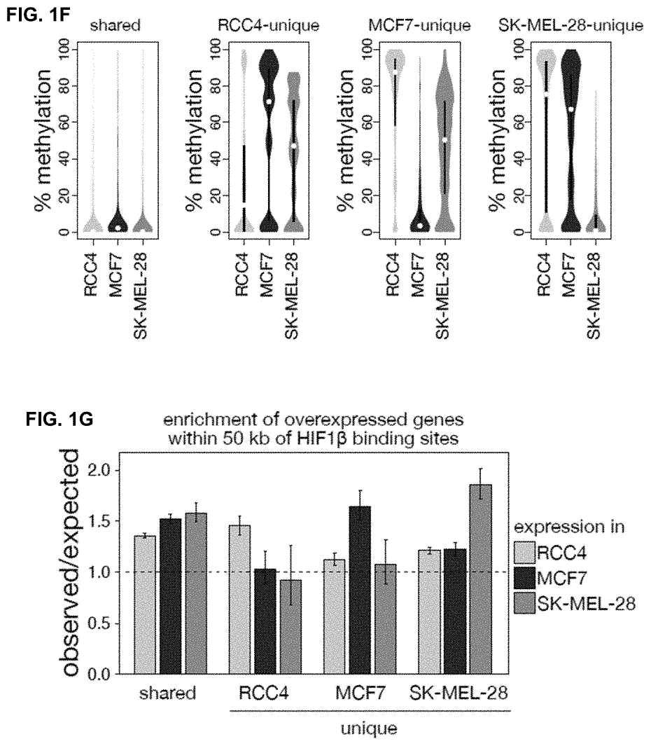

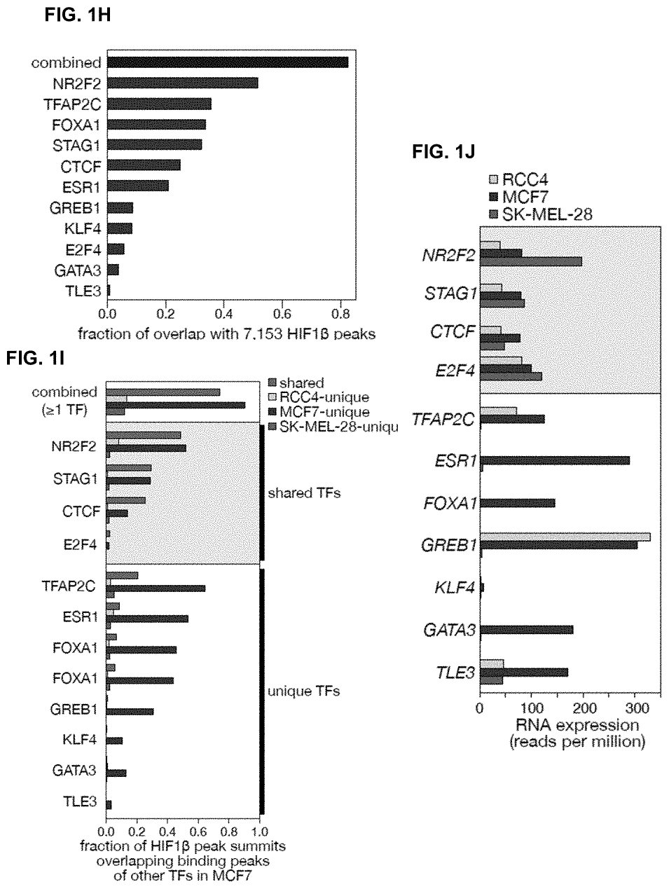

To investigate the role of DNA methylation in HIF binding, we stabilized HIFs in MCF7 breast cancer cells by culturing them under hypoxia (0.5% O₂ for 16 hours). We next performed chromatin-immunoprecipitation coupled to high-throughput sequencing (ChIP-seq) for HIF1β, which is the obligate dimerization partner of HIF1α, HIF2α and HIF3a. Model-based analysis for ChIP-seq (MACS) (Feng et al. 2011, Curr Protoc Bioinformatics Chapter 2, Unit 2 14) revealed 7,153 HIF1β binding peaks (FIG. 1A). These were high-quality, bona fide HIF binding regions: they were 4.6-fold enriched for the HRE motif (RCGTG), enriched near genes involved in the hypoxia response, >90% overlapping with peaks identified in another HIF1β ChIP-seq dataset on MCF7 cells (Schodel et al. 2011, Blood 117:e207-e217) and reproducibly detected in independent repeats.

To assess methylation in these 7,153 HIF1β binding peaks, we performed target enrichment-based bisulfite sequencing (BS-seq) on DNA extracted from normoxic MCF7 cells, in which HIF is inactive, obtaining >40× coverage for ˜86% of the HIF1β binding peaks identified by ChIP-seq. The methylation level at these peaks was invariably low (4.95±0.15%) compared to the average CpG methylation level detected in the genome (61.6±0.07%, Wilcoxon test P<2.2\hat{ }-16, FIG. 1B). Results were confirmed using another whole-genome BS-seq dataset (FIG. 1A) (Menafra et al. 2014, PLoS One 9:e99603). Also when quantifying methylation across all RCGTG motifs, including those located outside of HIF1β binding peaks, the inverse correlation between DNA methylation and HIF binding was confirmed (FIG. 1C). As BS-seq does not discriminate between 5-methyl and 5-hydroxymethylcytosine (Huang et al. 2010, PLoS One 5:e8888), we confirmed by DNA immunoprecipitation with an antibody recognizing only 5mC (5mC-DIP-seq) that HIF1β binding peaks were 6-fold depleted in 5mC-DIP-seq reads (FIG. 1A).

Moreover, methylation analysis of normoxic HIF1B-knockout MCF7 cells (Ahmed et al. 2013, Toxicol Sci 138:89-103) revealed identical methylation patterns, indicating that the unmethylated state of HIF1β binding sites is not due to baseline activities of HIF1β under normoxia. Importantly, identical results were obtained for murine embryonic stem cells (ESCs): the loci corresponding to the 4,794 HIF1β binding sites identified in wild-type ESCs were unmethylated in normoxia, and this both in wild-type and Hif1b-knockout ESCs (Maltepe et al. 1997, Nature 386:403). Since cells were intentionally exposed only briefly to hypoxia (16 hours), which fails to induce pronounced DNA methylation changes (Thienpont et al. 2016, Nature 537:63-68), these data suggest that regions to which HIF1β binds upon hypoxia are devoid of DNA methylation under normoxic conditions.

### Example 2.2. Cell-Type-Specific DNA Methylation of HREs Determines HIF Binding

Different cell types respond differently to hypoxia. To assess whether cell-type-specific DNA methylation could underlie this phenomenon, we profiled DNA methylation and HIF1β binding in 2 additional cell lines (RCC4 and SK-MEL-28). 20,613 HIF1β binding peak positions were detected across these cell lines. For each cell line, HIF1β binding was annotated as ‘present’ if the peak area showed >4-fold enrichment over the local read depth, and as ‘absent’ if it showed <2.5-fold enrichment; intermediate enrichment was annotated as unclassified. When comparing cell lines using these criteria, HIF1β binding was shared by all 3 cell lines at 6,152 sites, and unique for an individual cell line at 7,140 sites (437, 1,193 and 5,510 unique sites, respectively for RCC4, MCF7 and SK-MEL-28) (FIG. 1D).

Crucially, when assessing DNA methylation, HIF1β binding peaks unique to individual cell lines were unmethylated in cells where the binding site was active, while active HIF1β binding peaks shared between all cell lines were unmethylated in all cell lines (FIGS. 1E-1F). This strict correlation suggests that DNA methylation underlies the cell-type-specific response to hypoxia. Differences in DNA methylation and concomitant HIF binding appeared functional, as transcriptome profiling under normoxic and hypoxic conditions revealed that genes with a flanking HIF1β binding peak unique to one cell line, were more frequently increased in expression under hypoxia in that cell line (FIG. 1G).

### Example 2.3. DNA Methylation Determines HIF Binding Independently of Other Chromatin Marks

To analyse whether other epigenetic modifications similarly correlate with HIF binding, we analysed ENCODE data publicly available for MCF7 cells (ENCODE Project Consortium 2012, Nature 489:57-74) (no data are available for RCC4 and SK-MEL-28). Particularly, we investigated marks of heterochromatin (H3K9me3, H3K27me3), active promoters (H3K4me3, H3K9ac, H3K14ac), active enhancers (H3K4me1, H3K27ac), open chromatin (FAIRE) and active transcription (RNA Pol II). Although some histone marks were enriched in a subset of HIF1β binding peaks, none were consistently found at all active HIF1β binding peaks, especially when looking outside of CpG islands. The previously reported co-occupancy with RNA polymerase II or open chromatin was also not consistently found at all active HIF1β binding peaks (Schodel et al. 2011, Blood 117:e207-e217; Xia & Kung 2009, Genome Biol 10:R113). This was confirmed in linear regression analyses assessing how each of these marks individually predicts HIF1β binding in MCF7 cells. DNA methylation (R²=0.43) outperformed all other marks, with marks of active chromatin such as RNA polymerase II occupancy, H3K4me3, open chromatin and H3K27ac showing poor correlations (R² resp. 0.11, 0.11, 0.04 and 0.04). When combining all marks in one model, the total R² was 0.47, with DNA methylation contributing to 67.5% of the predictive power (partial R²=0.32). In line with this, omitting DNA methylation from the model reduced the total R² by more than half, to 0.21. We also assessed whether more general differences in chromatin states (using ChromHMM—Taberlay et al. 2014, Genome Res 24:1421-1432) underlie differential HIF binding. This revealed that while shared HIF1β binding sites were more frequent in promoters, sites unique to MCF7 were more frequent in enhancers, and sites inactive in MCF7 (but unique to RCC4 or SK-MEL-28) more frequent in MCF7-repressed chromatin. In line with enrichment at open chromatin, HIF1β binding thus appears exclusive to active enhancers and promoters while depleted in areas of repressed chromatin. Finally, NOMe-seq data from MCF7 cells revealed that, while open chromatin regions were generally unmethylated, a significant fraction of open chromatin (7-19%) in fact showed methylation, providing a potential rationale for the relatively small contribution of open chromatin in predicting HIF1β binding. Combined, these data show that poised HIF binding sites are in unmethylated regions that consist mostly of active, open chromatin, but are not consistently marked by other epigenetic modifications, in normoxia.

### Example 2.4. Other Transcription Factors (TFs) Determine the Methylation Landscape to Guide HIF Binding

Interestingly, many of the HIF1β binding peaks overlapped with binding sites for other TFs. Specifically, out of the 7,153 HIF1β binding peaks detected in MCF7 cells, 5,903 overlapped with the binding site of at least one TF (83%), out of a set of 11 TFs for which genome-wide binding site data were available in MCF7 cells (Griffon et al. 2015, Nucleic Acids Res 43:e27) (FIG. 1H). This could indicate that these TFs, being already active under normoxic conditions, drive demethylation of HIF1β binding regions (Feldmann et al. 2013, PLoS Genet 9:e1003994; Stadler et al. 2011, Nature 480:490-495), thus setting the stage for HIF binding upon hypoxia. To further support this notion, we also assessed binding of these 11 TFs at HIF1β binding peaks identified in RCC4 and SK-MEL-28 cells. Interestingly, TFs expressed by the 3 cell lines (e.g. CTCF or STAG1) co-localize in their binding with the shared HIF1β binding peaks. TFs only expressed in MCF7 cells (e.g. ESR1 or GATA3) overlap in their binding sites only with MCF7-specific HIF1β binding peaks. Finally, HIF1β binding peaks unique to RCC4 or SK-MEL-28 did not overlap with the binding sites of these 11 TFs in MCF7 (FIGS. 1I-1F). Differential expression and binding of TFs between different cells is thus likely to shape the DNA methylation landscape and determine subsequent HIF binding.

### Example 2.5. DNA Methylation does not Determine Differential Binding of HIF1a and HIF2α

Comparison of our 7,153 HIF1β peaks to previously published HIF1a and HIF2α ChIP-seq data in MCF7 cells (Schodel et al. 2011, Blood 117:e207-e217) revealed the methylation status of HIF1β binding peaks to be independent of the HIFα binding partner. Remarkably however, there were differences in the chromatin profiles of HIF1α- and HIF2α-bound regions: HIF1a binding sites showed 1.37-fold higher average levels of the promoter mark H3K4me3, whereas levels of the enhancer mark H3K4me1 were 0.75-fold lower at HIF1a binding sites than at HIF2α sites. Similarly, chromHMM analysis showed enrichment of HIF1a at promoters and depletion at enhancers relative to HIF2α. Moreover, other TFs similarly differed in occupancy between HIF1α- and HIF2α-specific sites: HIF2α was enriched at MCF7-specific TF binding sites, (which mostly correspond to cell-type-specific enhancers), and TFs shared between MCF7, RCC4 and SK-MEL-28 showed no enrichment of binding between HIF1a and HIF2α target sites. In conclusion, HIF1a preferentially binds at promoters, and HIF2α at enhancers, but DNA methylation differences do not determine their binding specificities.

### Example 2.6. DNA Methylation Directly Repels HIF Binding in Cells

To more firmly establish a causal link between DNA methylation and HIF binding, we excluded several confounders. Firstly, since our chromatin state analysis revealed that HIF preferentially binds active enhancers and promoters, which are known to carry low levels of methylation (Schubeler 2015, Nature 517:321-326), we performed HIF1β ChIP-bisulfite sequencing (HIF1β ChIP-BS-seq). MCF7 cells were exposed to hypoxia, HIF1β-bound DNA was immunoprecipitated and bisulfite-converted prior to sequencing to uncover its methylation pattern. This revealed that, while methylation levels of input DNA (not immunoprecipitated, bisulfite-converted DNA) were mostly low but with some sites displaying intermediate to high methylation levels, HIF1β-bound DNA was invariably very low in methylation and this at all sites (FIG. 2A).

Secondly, since TFs can drive demethylation of their binding sites both passively or actively, we excluded the possibility that DNA fragments bound by HIF would undergo DNA demethylation upon HIF binding. Indeed, HIF1β has previously been shown to actively recruit DNA demethylases (Mariani et al. 2014, Cell Rep 7:1343-1352). However, HIF1β ChIP-BS-seq in hypoxic murine ESCs deficient for all DNA demethylases (Tet1, Tet2 and Tet3) showed results identical to those observed in wild-type MCF7 cells: HIF1β-bound DNA was unmethylated compared to input DNA subjected to whole-genome BS-seq (FIG. 2B).

Additionally, other (unknown) confounders related to the binding location of HIF, such as chromatin environment or sequence context, may contribute to preferential HIF binding to unmethylated DNA. To exclude this possibility, we generated isogenic murine ES cell lines in which a human HIF1β binding site-encoding DNA fragment was inserted that was either in vitro methylated or not (FIG. 2C). Following recombination, the difference in methylation state between both fragments was maintained. HIF1β ChIP-qPCR revealed that methylation was sufficient to induce a 12.4-fold reduction in HIF1β binding in these isogenic cell lines (FIG. 2D).

Finally, to directly assess methylation sensitivity of HIF binding to unchromatinized DNA, we employed microscale thermophoresis, and tested the binding of recombinant co-purified HIF1α-HIF1β and HIF2α-HIF1β heterodimers to double-stranded DNA oligonucleotides containing a methylated or unmethylated RCGTG motif. Importantly, HIF1a and HIF2α containing heterodimers both showed a 15-fold higher affinity (K_{D}) for an unmethylated than methylated RCGTG motif, thus confirming that methylation directly repels binding of HIF1α-HIF1β and HIF2α-HIF1β heterodimers (FIGS. 2E-2F). Indeed, leveraging the crystal structure of the HIF1α-HIF1β and the HIF2α-HIF1β complexes bound to DNA (Wu et al. 2015, Nature 524:303-308), revealed that both cytosines in the CpG dinucleotide of the HIF binding sequence are snuggly accommodated via van der Waals interactions with the guanidine groups of Arg102 in HIF1β and Arg27 in HIF1a or HIF2α, respectively (Wu et al. 2015, Nature 524:303-308). Methylation of any of the two cytosines either on the top or bottom strand would in a static model drastically violate the minimum 3.1 Ångström length of van der Waals radii, and would be poised to cause severe steric clashes with these two functionally important arginine residues.

### Example 2.7. DNA Demethylation Enables Ectopic HIF Binding

Next, we investigated which parts of the genome are protected from HIF binding by DNA methylation. For this, we compared HIF1β binding in hypoxic wild-type murine ESCs versus ESCs deficient for DNA methyltransferases (Dnmt-triple-knockout or Dnmt-TKO), which lack DNA methylation (Tsumura et al. 2006, Genes Cells 11:805-814). This revealed a marked increase in the number of HIF1β binding peaks, from 2,676 in wild-type to 6,964 in Dnmt-TKO ESCs (FIG. 3A). Whole-genome BS-seq further revealed that, while shared binding peaks were unmethylated in both cell lines, Dnmt-TKO-specific HIF1β binding peaks had high methylation levels in wild-type ESCs, as expected (FIG. 3B).

All shared binding peaks were associated with a similar enrichment of the RCGTG motif (FIG. 3C), as well as with genes that were induced upon hypoxia (FIG. 3D). However, Dnmt-TKO-specific sites were more often distal to annotated transcription start sites, and more frequently in repressed chromatin regions of wild-type ESCs (FIGS. 3E-3G). Gene ontology analysis moreover failed to identify enrichment of hypoxia-related processes for Dnmt-TKO-specific binding peaks, in contrast to shared peaks (FIG. 3H). Thus, the majority of these Dnmt-TKO-specific binding peaks represent ectopic binding events.

### Example 2.8. DNA Methylation Represses Hypoxia-Induced Expression of Retrotransposons

Remarkably, a substantial fraction of novel Dnmt-TKO-specific HIF1β binding peaks were found in repetitive genomic regions. Particularly, repeat class analysis revealed a 2.8-fold increase in binding peaks on retrotransposons (280 of 2,487 (11.3%) shared peaks versus 1,468 of 4,477 (32.8%) Dnmt-TKO-specific peaks). The bulk of this increase was ascribable to binding at the 5′ of the long terminal repeat (LTR) family of endogenous retrovirus K (ERVK) sequences, with 786 of 1,468 binding peaks being at ERVKs (FIG. 4A). While HIF1β binding was also observed at LINEs and SINEs, binding at LTRs was enriched over a random permutation of HIF1β binding sites, and this was seen both for binding events proximal and distal to transcription start sites (FIG. 4B). Given that these analyses rely on uniquely mapping reads, which are inherently depleted of repetitive DNA, this enrichment is likely to represent an underestimate. We also assessed whether a similar phenomenon is at play in human cancer cell lines, and pharmacologically demethylated MCF7 cells using 5-aza-2′-deoxycytidine (aza), overall reducing DNA methylation by 70.5±5.5%. HIF1β ChIP-seq revealed that aza exposed 1,236 new HIF1β binding peaks. These new binding sites were methylated in untreated MCF7 cells and showed a 2.5-fold reduced methylation in aza-treated cells. While HIF1β binding peaks in retrotransposons were already present in vehicle-treated MCF7 cells, novel aza-specific HIF1β binding peaks were 2-fold enriched for retrotransposons (8.1% versus 4.1%, respectively). Aza-specific HIF1β binding peaks were enriched in all three retrotransposon classes, i.e. LINEs, LTRs and SINEs, with at least 10 out of 13 retrotransposon families bound by HIF1β in untreated MCF7 cells being enriched for HIF1β binding after aza and hypoxia (FIG. 4C).

Notably, different retrotransposons were affected in human MCF7 cells compared to murine ESCs due to the evolutionary divergent repeat content of these genomes. An analysis of the distribution of HIF1β binding peaks at retrotransposons, however, revealed that HIF1β binding sites were at the 5′ end of retrotransposon sequences, and that patterns of binding were conserved between mouse and human genomes, suggesting that HIF binding on retrotransposons is functional.

To confirm the latter, we applied mRNA-seq followed by RepEnrich analysis (Criscione et al. 2014, BMC Genomics 15:583) to assess changes in retrotransposon expression following 24 hours of hypoxia with or without aza. When focusing on the differential expression of retrotransposon subfamilies (5% FDR), we found that, already under hypoxia alone, 251 of all LTR (44%), 51 of all LINE (32%) and 5 of all SINE (10%) subfamilies were upregulated, while only 16 LTR, 7 LINE and no SINE subfamilies were downregulated (FIGS. 4D-4E). Upregulated retrotransposons were moreover 2.8-fold enriched in HIF1β binding peaks when comparing to retrotransposons not upregulated by hypoxia (χ2-test P<2.2×10\hat{ }-16). In line with this, when specifically assessing whether HIF1β-bound retrotransposons were also differentially expressed, we observed that nearly all retrotransposon families bound by HIF1β exhibited increased expression (FIG. 4D). At the subfamily level, expression analysis of the 176 subfamilies bound by HIF1β revealed that both aza and hypoxia alone increased expression (23% and 12%, respectively), whereas combined aza and hypoxia induced the highest increase (27%; FIGS. 4E-4F). These changes were even more pronounced after a chronic (4-day) exposure to hypoxia, as retrotransposons were on average 16% or 45% increased following hypoxia alone or combined aza and hypoxia. To confirm that retrotransposon subfamily expression was dependent on HIF, we assessed their expression in HIF1B-knockout MCF7 cells. This failed to upregulate retrotransposons in hypoxia (7% decrease), while retaining aza-induced retrotransposon overexpression (25% increase). Hypoxia also failed to further increase the effect of aza (25% for aza alone versus 20% for aza in hypoxia). Finally, pharmacological activation of HIF using dimethyloxalylglycine (Elvidge et al. 2006, J Biol Chem 281:15215-15226) affected retrotransposon expression similar to hypoxia. Combined, our data indicate that hypoxia triggers HIF binding to unmethylated retrotransposons and activates their expression in a HIF-dependent manner.

### Example 2.9. Hypoxia and Retrotransposon Expression Affect Tumour Immunotolerance

Retrotransposon expression has been linked to tumour foreignness (Blank et al. 2016, Science 352:658-660), interferon response (Chiappinelli et al. 2015, Cell 162:974-986; Zitvogel et al. 2015, Nat Rev Immunol 15:405-414) and enhanced cytolytic activity (Rooney et al. 2015, Cell 160:48-61), all critical determinants of response to cancer immunotherapy. This suggests that retrotransposon expression induced by HIF could contribute to an immune-activated microenvironment. To study this in more detail, we reanalysed gene expression and DNA methylation data from The Cancer Genome Atlas (TCGA). We classified 4,494 tumours from 14 cancer types as hypoxic or normoxic using an established hypoxia metagene expression signature (Buffa et al. 2010, Br J Cancer 102:428-435), and used RepEnrich to remap all RNA-seq reads and determine expression of the 779 retrotransposon subfamilies.

While these tumours were not exposed to DNA demethylating agents, they did show variation in DNA methylation at retrotransposons. Indeed, although CpGs in retrotransposons showed mostly high methylation levels (median=80.7%), there was considerable variability (9.2% standard deviation), and one in 10 tumours displayed median levels below 67.3%. Remarkably, and in line with our in vitro data, there was a significant interaction between hypoxia and DNA methylation in determining retrotransposon expression (P=0.019), with expression being increased in hypoxic tumours having lower methylation at retrotransposons (FIGS. 5A-5B). This interaction was particularly striking in cancer types known to respond to immunotherapy (Turajlic et al. 2017, Lancet Oncol 18:1009-1021) (P=0.0002 in responsive versus P=0.796 in non-responsive tumours). As expected, responsive cancer types had a higher mutation load, increased immune checkpoint expression, more CD8⁺ T cells and increased cytolytic activity (FIG. 7A). Importantly, responsive types also had on average lower methylation at retrotransposons, and higher retrotransposon expression than non-responsive types (P<10″-16 for both comparisons, FIG. 5C). In line with our in vitro findings, DNA methylation could thus underlie retrotransposon expression in hypoxic tumours.

A reanalysis of ribosome profiling data moreover demonstrated that retrotransposons were characterized by a polysome:monosome enrichment that was similar to coding genes but not to non-coding genes (Bai et al. 2016, Nat Commun 7:12310). This suggests that retrotransposons are translated and based on earlier reports that retrotransposons can be antigenic (Kassiotis & Stoye 2016, Nat Rev Immunol 16:207), may encode neo-epitopes (FIG. 5D). In line with this notion, there was a significant correlation between retrotransposon expression and immune checkpoints, such as PD1 (P=0.0081) and LAG3 (P=0.0031), and an inverse correlation with B2M expression (P=0.0103), and this independently of CD8+ T-cell infiltration estimates and hypoxia (FIG. 7A).

Interestingly, of the 59 retrotransposons whose expression significantly correlated with cytolytic activity in responsive tumours (5% FDR), most were also upregulated in hypoxic or aza-treated MCF7 cells (P<0.05) (FIG. 5E), thereby underlining the role of hypoxia and DNA methylation in regulating expression of potentially immunogenic retrotransposons. Overall, these observations support a model wherein hypoxia-induced retrotransposon expression is tolerated in high-immunogenic tumours, as these are characterized by high immune checkpoint expression, but not in low-immunogenic tumours where their expression would compromise tumour immunotolerance. This also suggests that low-immunogenic tumours may need to maintain high DNA methylation levels in retrotransposons to downregulate their expression and avoid the induction of tumour immunogenicity.

### Example 2.10. Aza Compromises Tumour Immunotolerance in Mice Via HIF

To confirm that retrotransposon expression can indeed compromise immunotolerance in low-immunogenic tumours, we explored several murine tumour models (4T1, B16, MC38 and CT26) for their immunogenicity, and identified the orthotopic 4T1 breast cancer model as low-immunogenic. Indeed, 4T1 tumours exhibited a low mutation burden, cytolytic activity, number of CD8+ T cells and expression of immune checkpoints (Pd1, PdI1) compared to B16, MC38 and CT26 models (FIG. 7B). In line with 4T1 being a low-immunogenic tumour, anti-PD1 treatment failed to affect 4T1 tumour growth (−8%, P=0.397), while reducing growth of high-immunogenic MC38 and CT26 tumours, as described previously (Kim et al. 2014, J Immunother Cancer 2:P267; Ngiow et al. 2015, Cancer Res 75:3800-3811) (−28% and −65%; P=0.030 and 0.023, respectively; FIG. 6A. Importantly, also retrotransposon expression was lower in 4T1 than in MC38 and CT26 tumours (FIG. 7B).

We next verified if in these low-immunogenic tumours DNA demethylation upregulates retrotransposons in a HIF-dependent manner. Importantly, we observed that, similar to MCF7 cells, both hypoxia and aza independently increase retrotransposon expression in 4T1 cells in vitro. Likewise, aza increased retrotransposon expression in vivo (FIG. 6B). To confirm that this upregulation was at least partially hypoxia-mediated, we investigated whether tumour hypoxia enhances aza-induced retrotransposon expression. We compared aza-treated 4T1 tumour-bearing mice injected either with control or anti-VEGFR-2 antibody (DC101). While vehicle-treated 4T1 tumours were hypoxic in ˜40% of the tumour, DC101 further reduced blood vessel density (−35%; P<0.05) while increasing hypoxic tumour areas (68%; P<0.05; FIGS. 7C-7D. Importantly, this was associated with increased retrotransposon expression (+9%; P=2.6×10\hat{ }-16; FIG. 7E).

Next, we explored if the increase in retrotransposon expression compromised immunotolerance. Aza treatment reduced growth of 4T1 tumours (−32%; P=3.0×10\hat{ }-3; FIG. 6C), but did not reduce cell proliferation marker expression. In contrast, immune activation was enhanced in tumours treated with aza, as activated T cell and natural killer cell signatures were upregulated and myeloid-derived suppressor cell signatures downregulated (FIG. 6D). Immunofluorescence of CD8+ T cells confirmed these changes: while T-cell infiltration was unaffected, the number of activated, granzyme B-positive T cells increased by 2.1-fold (P<0.05) (FIG. 7F). Interestingly, previous experiments have shown that PyMT breast tumours, which also classify as low-immunogenic tumours (FIG. 7B), are also resistant to immunotherapy (Allen et al. 2017, Sci Transl Med 9(385): eaak9679; Schmittnaegel et al. 2017, Sci Transl Med 9(385): eaak9670), but sensitive to DNA demethylation (Chen et al. 2012, Mol Cancer Ther 11:370-382).

Finally, to verify HIF-dependence of these effects, we generated 4T1 cells deficient for HIF1β by CRISPR-Cas9 (4T1^{Hif1b-KO}) and compared these cells to scramble-control 4T1 cells (4T1^{Hif1b-scr}), while treating with aza or vehicle. In vitro, loss of HIF1β in 4T1 cells abrogated hypoxia-induced retrotransposon expression. Likewise, 4T1^{Hif1b-scr} grafts showed higher retrotransposon expression than 4T1^{Hif1b-KO} grafts (FIG. 6E). Importantly, while 4T1^{Hif1b-scr} grafts also showed a significantly reduced size when comparing aza to vehicle (P=0.021), 4T1^{Hif1b-KO} failed to show this reduction (P=0.21; FIG. 6F). Of note, 4T1^{Hif1b-KO} tumours grew slower than 4T1^{Hif1b-scr} tumours, rendering disentanglement of cell proliferation-dependent and -independent effects difficult. Nevertheless, aza treatment induced a similar and significant upregulation of cancer testis antigen expression in both lines, suggesting similar treatment efficacy. Moreover, while the number of activated T cells increased in 4T1^{Hif1b-scr} grafts following aza, 4T1^{Hif1b-KO} grafts failed to show such increase (FIG. 6G). Together, these data provide a mechanistic link between HIF1β binding, DNA methylation and immune activation, highlighting the potential for DNA methylation inhibitors to activate the immune system and render immune-cold tumours immune-hot.

### Example 2.11. Discussion Examples 2.1-2.10

Here, we show that DNA methylation directly repels binding of HIF transcription factors and that cell-type-specific DNA methylation patterns established under normoxic conditions underlie the differential hypoxic response between cell types. Furthermore, ectopic HIF binding sites enriched at retrotransposons are normally masked by DNA methylation but become accessible to HIF upon DNA demethylation, thereby upregulating their expression and enhancing tumour immunogenicity. We show that also in human tumours, even without being exposed to DNA methylation inhibitors, HIF binding sites in retrotransposons can be unmethylated, especially in tumours with high immune checkpoint expression. This suggests that hypoxia-induced retrotransposon expression is tolerated in high-immunogenic tumours, but not in low-immunogenic tumours where their expression could compromise tumour immunotolerance. By confirming the latter in a low-immunogenic mouse tumour model, we propose DNA methylation to act as a regulator of immunotolerance in hypoxic tumours.

Our findings are of importance for a number of reasons. Firstly, an instructive role of DNA methylation in gene expression regulation, as originally proposed by Holliday and Pugh and by Riggs (Riggs 1975, Cytogenet Cell Genet 14:9-25; Holliday & Pugh 1975, Science 187:226-232), has remained controversial. Indeed, in many instances it is unclear if DNA methylation changes are a direct or indirect cause, or perhaps a consequence of changes in transcription factor binding or gene expression (Schubeler 2015, Nature 517:321-326). This uncertainty limits the impact of DNA methylation profiling to improve our understanding of (tumour) cell biology: if DNA methylation changes are simply secondary to other alterations, then therapeutic targeting of the DNA methylome may be relatively ineffective. By demonstrating that DNA methylation directly repels HIF binding, and by inference can influence binding of other TFs, the therapeutic potential and working mechanism of DNA methylation inhibitors may have to be revised. Specifically, tumour hypoxia has long been associated with increased malignancy, poor prognosis and resistance to radio- and chemotherapy (Keith et al. 2012, Nat Rev Cancer 12:9-22). Our understanding of how genes are modulated by tumour hypoxia through the epigenome provides important insights in the processes underlying therapeutic resistance. For instance, Vanharanta and colleagues recently showed an association between DNA methylation near CYTIP, and the survival of disseminating cancer cells (Vanharanta et al. 2013, Nat Med 19:50-56). Combined with our observations that DNA methylation directly repels HIF binding, this suggests remethylation of the CYTIP promoter as a viable avenue for decreasing cancer dissemination. Secondly, although hypoxic responses are evolutionarily conserved, not all cells respond identically to hypoxia (Chi et al. 2006, PLoS Med 3:e47). It has indeed been challenging to identify a guiding principle as to why specific genes are induced upon hypoxia in one, but not in the other cell type. Our findings suggest that cell-type-specific TF binding under normoxia causes differences in DNA methylation, which determine HIF binding under hypoxia and predict the cell-type specific hypoxia response. Importantly, we confirmed earlier observations that HIF1β binding peaks are characterized by an active, open chromatin structure (Xia & Kung 2009, Genome Biol 10:R113). This additional requirement for functional HIF1β binding peaks probably explains why each of the RCGTG consensus sequences that are present in human or murine genomes cannot serve as an equal HIF binding substrate in normal cells, or upon genetic or pharmacological demethylation.

Similar observations were made for CTCF and other TFs: binding of these methylation-insensitive TFs was similarly limited to sites containing a permissive chromatin structure (Stadler et al. 2011, Nature 480:490-495; Maurano et al. 2015, Cell Rep 12:1184-1195). Importantly, binding specificities for HIF1a versus HIF2α are independent of DNA methylation but appear to be influenced by chromatin context. This is in line with the identical structure of DNA binding domains of HIF1a and HIF2α; swapping DNA binding domains between both proteins has no influence on their binding profile (Partch & Gardner 2011, Proc Natl Acad Sci USA 108:7739-7744). Instead, the transactivation domain appears to endow specificity, indicating that accessory binding partners are required to trigger the differential binding of HIF1a and HIF2α (Partch & Gardner 2011, Proc Natl Acad Sci USA 108:7739-7744).

Finally, we also uncover an intriguing opportunity for cancer immunotherapy. Several publications have reported how 5-aza-2′-deoxycytidine initiates cryptic start sites in the repeat genome, leading to retrotransposon overexpression (Brocks et al. 2017, Nature Genetics 49:1052; Chiappinelli et al. 2015, Cell 162:974-986). Chiapinelli et al. demonstrated that aza-induced retrotransposons are highly immunogenic and can sensitize tumours to checkpoint immunotherapy (Chiappinelli et al. 2015, Cell 162:974-986), while Cheng et al. showed that also histone demethylase LSD1-ablation increases repetitive element expression, thereby enabling checkpoint blockade (Sheng et al. 2018, Cell 174:549-563). We demonstrate that retrotransposon expression is at least partly HIF-dependent, but more importantly, that also hypoxia alone (independently of drugs targeting the epigenome) is capable of inducing expression of retrotransposons with an unmethylated HIF binding site. Although the effect after 24 hours of hypoxia was only moderate compared to aza (12% versus 23% increase), retrotransposon expression further increased when MCF7 cells were, similar to aza, exposed to 4 days of hypoxia. Tumour hypoxia is also endemic to most human solid tumours, and therefore the described effects on retrotransposon expression could have a widespread impact. Indeed, in hypoxic tumours with high checkpoint expression, DNA methylation at retrotransposons was reduced and consequently, hypoxia-induced retrotransposon expression increased. Since tumours with high checkpoint expression often respond to checkpoint immunotherapy, and as retrotransposons could sensitize tumours to checkpoint blockade (Chiappinelli et al. 2015, Cell 162:974-986), this suggests hypoxia-induced retrotransposons to play an important role in mediating the therapeutic effects exerted by immune checkpoint blockade. In contrast, immune-cold tumours characterized by low checkpoint expression were much less permissive to retrotransposons, showing high methylation at retrotransposons and reduced retrotransposon expression. In light of our findings that methylation directly repels HIF binding, this suggests DNA methylation to block hypoxia-induced retrotransposon expression in immune-cold tumours to maintain immunotolerance. Pharmacological demethylation of immune-cold 4T1 tumours indeed increased retrotransposon expression, enhanced immunogenicity and reduced tumour growth in a HIF-dependent manner. Immune-cold tumours are typically non-responsive to immune checkpoint blockade. By showing that low-immunogenic, hypoxic tumours can be rendered vulnerable to the immune system through DNA methylation inhibition, we thus highlight a novel treatment strategy for tumours otherwise resistant to immunotherapies.

### Example 2.12. De Novo Retrotransposon Expression Analysis in Samples of Melanoma Patient Treated with Immune Checkpoint Inhibitor, 1^{st} Round

2.12.1. Patient Cohorts

Three melanoma cohorts consisting of a total of 90 melanoma tumors taken from 71 patients were used as basis for the analysis. All patients were treated with the immune checkpoint PD-1 inhibitor nivolumab and had no previous treatment for any other immunotherapy. 72 tumor biopsies were taken before nivolumab treatment and 18 of them have paired on-therapy samples. RNA-sequencing of these samples was performed. For each patient, overall survival (OS) and response to treatment, as assessed by RECIST were collected. In the Hugo et al. 2016 (Cell 165:35-44) data set, two biopsies were taken from different locations on the same patient and sequenced independently; these were treated as two independent samples in the survival analysis. Sample numbers are shown in Table 1 below. Additionally, Riaz et al. 2017 (Cell 171:934-949) also collected on-treatment tumor biopsies. Data available were therefore also assessed as pre-treatment/on-treatment tumors pairs, as listed in Table 2.

RECIST, or “Response Evaluation Criteria In Solid Tumors” is a standard way to measure response of a cancer patient to treatment (does tumor shrink, is it stable, or does it grow?). To use RECIST, there must be at least one tumor that can be measured on x-rays, CT scans, or MRI scans. The types of response a patient can have are a complete response (CR), a partial response (PR), progressive disease (PD), and stable disease (SD).

TABLE 1
Sample number summary.

| Cohort | | own | “Hugo” | “Riaz” |
|---|---|---|---|---|
| Source | | unpublished | Hugo et al. | Riaz et al. |
| | | | 2016, Cell | 2017, Cell |
| | | | 165: 35-44 | 171: 934-949 |
| Total patient number | | 19 | 28 | 25 |
| RECIST | PD | 5 | 13 | 12 |
| | SD | 3 | 0 | 5 |
| | PR | 7 | 5 | 4 |
| | CR | 4 | 10 | 2 |
| Overall | Unknown | 0 | 0 | 2 |
| survival | Censored | 14 | 16 | 7 |
| | Dead | 5 | 12 | 18 |

| | | | |
|---|---|---|---|
| function of cohort | modelling | modelling | validation |
| in analysis | | | |

PD-progressive disease; SD-stable disease; PR-partial response; CR-complete response.

The own cohort will hereinafter be referred to as the “Leuven cohort”, the cohort described in Hugo et al. 2016, Cell 165:35-44 will be referred to as the “Hugo cohort”, and the cohort described in Riaz et al. 2017, Cell 171:934-949 will be referred to as the “Riaz cohort”.

TABLE 2
Pre-treatment/on-treatment paired sample number.

| RECIST | PD | SD | PR | CR |
|---|---|---|---|---|
| Number of pairs | 9 | 4 | 3 | 2 |

2.12.2. De Novo Retrotransposon Detection

First, sequencing reads obtained by RNA-sequencing of 472 publicly-available melanoma tumor samples in TCGA were assembled de novo. To this end, the aligned bam files from TCGA were downloaded, and de novo assembly was performed using the StringTie v1.3.4 software by applying the default parameter settings on each individual patient file. The known human gene annotation Ensembl92 was also fed into StringTie as guidance to the assembly process. All of the output (expressed reference transcripts and novel transcripts) GTF files were further integrated using the StringTie-merge function to generate a non-redundant set of transcripts. Among the known IncRNAs and newly detected transcripts resulting from the de novo transcript assembly, retrotransposons were singled out as exons overlapping with retrotransposons recorded in RepeatMasker (software that screens DNA sequences for interspersed repeats and low complexity DNA sequences). As result, a melanoma retrotransposon annotation was created. Next, the RNA-seq data from tumors of patients belonging to each of the 3 patient cohorts treated with checkpoint inhibitors (see above) were assessed. First, the fastq files were pre-processed by converting pair-end reads to single-end reads and by reducing long reads to 51 nucleotides (the Hugo et al. 2016 (Cell 165:35-44) data are 2×100 nucleotide reads). Then these data were aligned to the human genome (GRCh38) using STAR with a tolerance of two mismatches. At last, read numbers for retrotransposons were called based on the annotation build in TCGA and then calculated the number of Reads Per Kilobase of transcript, per Million mapped reads (RPKM), as a read-out of retrotransposon expression.

The human reference genome GRCh38 (Equivalent UCSC version hg38) was released from the Genome Reference Consortium (GRC) on 24 Dec. 2013. The previous human reference genome (GRCh37) was the nineteenth version. This build contained around 250 gaps, whereas the first version had roughly 150,000 gaps. The GRCh38 assembly saw the closure or reduction of more than 100 gaps.

Single-end RNA-seq results are often reported in RPKM (Reads Per Kilobase Million). This metric attempts to normalize for sequencing depth and gene length. First, the total reads in a sample are counted up, and that number is divided by 1,000,000—this is the “per million” scaling factor. Secondly, the read counts are divided by the “per million” scaling factor—this normalizes for sequencing depth, giving reads per million (RPM). Thirdly, the RPM values are divided by the length of the gene, in kilobases. This gives the RPKM value.

2.12.3. Differentially Expressed Retrotransposons in Leuven and Hugo Cohorts

First, DESeq (DESeq is an R package to analyse count data from high-throughput sequencing assays such as RNA-Seq and test for differential expression) was applied to identify all retrotransposons differentially expressed between patients displaying a treatment response (PR and CR) versus patients displaying no response (PD and SD) groups, in the Leuven cohort and the Hugo cohort, respectively (FIG. 8). Retrotransposons with P<0.05 and a log 2 fold change >2.5 were considered as differentially expressed (“DE”). Overall, retrotransposons were identified for which expression was high in response versus non-response patients, or vice versa. Of these, 30 retrotransposons (29 retrotransposons when omitting the retrotransposon located on the Y chromosome) were upregulated in patients with a treatment response in both cohorts, while only 2 retrotransposons were lower in responding patients. This bias was significant (two-tailed Fisher exact test, p=0.0003) (FIG. 9). The former 30 retrotransposons (29 retrotransposons when omitting the retrotransposon located on the Y chromosome) were considered as potential biomarkers.

Next, a method was developed to leverage these 30 retrotransposon (29 retrotransposons when omitting the retrotransposon located on the Y chromosome) biomarkers as a potential marker of efficacy of immune checkpoint therapy. First, a threshold was set that allowed to determine when a given retrotransposon could be considered as ‘being expressed’. Particularly, a cutoff of RPKM>0.25 was defined to consider a retrotransposon as being expressed. This cutoff is based on the elbow point of the RPKM distribution of the 30 retrotransposons in both cohorts (FIG. 10; similar distribution when omitting the Y chromosome-located retrotransposon). For every biomarker, this RPKM cutoff is still smaller than the maximum value among all modelling patients (between 0.27 and 15.0), thus, every biomarker is passing the cutoff for at least one patient. Second, a cutoff was established to consider a tumor sample as a high- or low-expresser of retrotransposons. For this threshold, the number of retrotransposons with a RPKM>0.25 were counted. According to the ROC curve (FIG. 11), the optimal threshold ranged between 2 to 4 retrotransposons. Therefore, we chose N=3 as the cut-off of expressed retrotransposons to consider a tumor sample as a high- or low-expresser of retrotransposons. A receiver operating characteristic curve, i.e., ROC curve, is a graphical plot that illustrates the diagnostic ability of a binary classifier system as its discrimination threshold is varied.

The signature of 30 retrotransposons with its defined thresholds was tested to predict overall survival (OS) in the Leuven- and Hugo cohorts (FIG. 12). In the Hugo cohort, significant OS differences between high- versus low-expressers of retrotransposons were observed (p<0.03; 24.33 versus 13.21 months). In the Leuven cohort, overall survival was longer in high-expressers of retrotransposons compared to low-expressers of retrotransposons, but effects were not significant (P=0.141; 110.2 versus 25.87 months), presumably due to the limited number of patients in this cohort and the limited follow-up available for some patients. When pooling both cohorts, survival differences predicted by the 29 retrotransposon gene signature were also significant (P=0.014; 29.83 months versus 16.01 months). This significance is not compromised when correcting for patient gender and age factors (P=0.011).

2.12.4. Validation of Differentially Expressed Retrotransposon Signature on Independent Riaz Cohort

In an effort to independently validate these observations, the identified retrotransposon expression signature was applied at the same thresholds to a third publicly-available and independently established cohort of 25 melanoma patients receiving nivolumab (Riaz et al. 2017, Cell 171:934-949). Differences on OS were analyzed based on high- versus low-expressers of retrotransposons (FIG. 13) and observed a significant difference (p=0.0398; median survival: 24.03 months versus 15.36 months) in high versus low-expressers of retrotransposons. Additionally, expression of these retrotransposons was compared in paired pre-treatment versus on-treatment tumor samples (FIG. 14). A significant down-regulation of retrotransposon expression was observed, and this only in patients with a response to checkpoint immunotherapy but not in those without a response (two-tailed Wilcoxon signed rank test, p<0.01). Tumor mutation burden is also established as a biomarker for checkpoint immunotherapy and the allelic frequency of mutation-carrying subclones is reduced during therapy, suggesting that similarly tumor clones expressing retrotransposons are under a negative selection bias during cancer immunotherapy.

2.12.5. Applying Retrotransposon Expression Biomarkers on all Three Patient Cohorts

Above results not only bring a useful signature to predict the immunotherapy outcomes in melanoma patients, but also showed the effectiveness of our methodology. In order to reinforce the signature using all three cohorts as a discovery set, rather than only two of them, we also performed DESeq tests for the response vs. non-response patients on different combinations. By applying the same criteria, we found 19 retrotransposons (17 when omitting the Y chromosome located retrotransposons) shared between Leuven and Riaz cohorts, and 9 retrotransposons (8 when omitting the Y chromosome located retrotransposons) shared between Hugo and Riaz cohorts. Furthermore, 2 retrotransposons are shared between all 3 patient cohorts (FIG. 15). Expression of these 19 and 9 retrotransposons was higher in responding versus non-responding patients. Interestingly, by generating a 19 retrotransposon and 9 retrotransposon-based signature, and by applying the same criteria to establish cut-offs for RPKM cutoff and number of retrotransposons expressed at RPKM>0.25, we could show that both signatures significantly predicted outcome in a pooled population (FIG. 16A, panel II and III). Similar results were obtained with the 17 retrotransposon and 8 retrotransposon-based signatures (FIG. 16B, panel II and III). Overall, we identified 3 signatures which predicted overall survival after anti-PD1 immunotherapy in melanoma. Together, these 3 signatures consist of 54 retrotransposons (50 when omitting the Y chromosome located retrotransposons). There coordinates within the human reference genome are listed in Table 3. For these unified 54 biomarkers, we found N-cutoff=3 (or 4) shows the best distinguishability on treatment responses through the similar ROC analysis (FIG. 17). Also, this unified signature shows significant prediction power on OS of all patients (p<0.01) (FIG. 16A, panel IV). Similar results were obtained with the 50 retrotransposon-based signature (FIG. 16B, panel IV).

TABLE 3
Retrotransposon biomarker coordinates in human genome GRCh38.

| | | | DE in¹ | | | Location in GRCh38 | | | |
|---|---|---|---|---|---|---|---|---|---|
| Gene ID | Ensembl ID | Retrotransposon³ | L | H | R | Chr | Start | End | Strand² |
| MSTRG.11332 | (Novel) | HERV9-int | 1 | 1 | 1 | 12 | 18041309 | 18046380 | − |
| | | (AluY) | | | | | | | |
| MSTRG.13715 | ENSG00000231238 | L1M4 | 1 | 0 | 1 | 13 | 18533646 | 18622853 | + |
| MSTRG.13742 | ENSG00000121388 | MSTA | 1 | 1 | 0 | 13 | 19181981 | 19189771 | + |
| | | (MSTA-int) | | | | | | | |
| MSTRG.14514 | (Novel) | MLT1G3 | 1 | 0 | 1 | 13 | 68025900 | 68037147 | + |
| MSTRG.14654 | (Novel) | MER57E1 | 0 | 1 | 1 | 13 | 81339706 | 81385324 | + |
| MSTRG.14671 | (Novel) | MER61-int | 1 | 1 | 0 | 13 | 83906092 | 84069354 | + |
| | | (MER61A) | | | | | | | |
| MSTRG.14675 | (Novel) | L1PB3 | 1 | 1 | 0 | 13 | 84086711 | 84449221 | + |
| MSTRG.15088 | (Novel) | AluSx3 | 1 | 0 | 1 | 14 | 19169024 | 19169266 | . |
| MSTRG.15434 | (Novel) | L1ME3Cz | 0 | 1 | 1 | 14 | 29109063 | 29109836 | . |
| MSTRG.16408 | (Novel) | L1MC4a | 1 | 0 | 1 | 14 | 83225538 | 83307805 | + |
| MSTRG.16413 | (Novel) | LTR16C | 0 | 1 | 1 | 14 | 83854257 | 83870210 | + |
| MSTRG.19710 | (Novel) | MIRb | 1 | 1 | 0 | 16 | 17831751 | 17868474 | − |
| | | (AluSz) | | | | | | | |
| MSTRG.19711 | (Novel) | L1PA17 | 1 | 1 | 0 | 16 | 17849826 | 17854608 | + |
| | | (MLT2E) | | | | | | | |
| MSTRG.19712 | (Novel) | MamGypsy2-I | 1 | 1 | 0 | 16 | 17857716 | 17859715 | + |
| MSTRG.24760 | (Novel) | L1PA5 | 1 | 1 | 0 | 18 | 28489199 | 28491505 | + |
| MSTRG.24775 | (Novel) | LTR1A2 | 1 | 1 | 0 | 18 | 30095986 | 30176042 | − |
| MSTRG.24811 | (Novel) | THE1A | 1 | 1 | 0 | 18 | 34264420 | 34331633 | − |
| MSTRG.25244 | (Novel) | THE1B | 1 | 1 | 0 | 18 | 66114175 | 66258594 | − |
| | | (AluYe5) | | | | | | | |
| MSTRG.28372 | (Novel) | MIRb | 1 | 0 | 1 | 2 | 5250772 | 5298264 | + |
| MSTRG.29165 | (Novel) | THE1C | 0 | 1 | 1 | 2 | 52313801 | 52394438 | − |
| MSTRG.32532 | ENSG00000229876 | L1ME4a | 1 | 1 | 0 | 20 | 6321979 | 6585670 | + |
| | | (L2a) | | | | | | | |
| MSTRG.32547 | (Novel) | LTR67B | 1 | 1 | 0 | 20 | 9884608 | 9889795 | − |
| MSTRG.33894 | (Novel) | L1MCa | 1 | 1 | 0 | 21 | 10617572 | 10621162 | . |
| MSTRG.34539 | (Novel) | L4_A_Mam | 1 | 1 | 0 | 22 | 10749242 | 10757355 | + |
| MSTRG.34540 | (Novel) | MLT1A1 | 1 | 1 | 0 | 22 | 11024745 | 11066134 | − |
| MSTRG.34546 | (Novel) | PRIMA41-int | 1 | 0 | 1 | 22 | 11559963 | 11570415 | + |
| MSTRG.37336 | ENSG00000242781 | ERVL-B4-int | 1 | 1 | 0 | 3 | 80764897 | 81096565 | + |
| MSTRG.37337 | (Novel) | L1PREC2 | 1 | 1 | 0 | 3 | 80871322 | 80876188 | + |
| MSTRG.38237 | (Novel) | L1MD1 | 1 | 1 | 0 | 3 | 145333834 | 145381982 | + |
| | | (L1M1) | | | | | | | |
| MSTRG.38351 | ENSG00000241151 | L2a | 0 | 1 | 1 | 3 | 151552073 | 151657966 | + |
| MSTRG.39697 | ENSG00000248215 | THE1D | 1 | 1 | 1 | 4 | 36496537 | 36641900 | − |
| MSTRG.41342 | (Novel) | THE1B | 1 | 0 | 1 | 4 | 166198265 | 166455907 | + |
| MSTRG.41489 | (Novel) | L1MC3 | 1 | 0 | 1 | 4 | 178312070 | 178313749 | . |
| MSTRG.42146 | ENSG00000251273 | MLT2C1 | 1 | 1 | 0 | 5 | 25087450 | 25190956 | − |
| MSTRG.42147 | ENSG00000245662 | L1PREC2 | 1 | 1 | 0 | 5 | 25186999 | 25324386 | + |
| MSTRG.42178 | (Novel) | L1MB8 | 1 | 1 | 0 | 5 | 28065058 | 28130814 | + |
| MSTRG.42183 | ENSG00000250453 | MLT1E2 | 1 | 1 | 0 | 5 | 28585921 | 28809249 | − |
| | | (MLT2B3) | | | | | | | |
| MSTRG.42187 | (Novel) | L1MB2 | 0 | 1 | 1 | 5 | 28813723 | 28816663 | . |
| MSTRG.42189 | (Novel) | L1MB4 | 1 | 1 | 0 | 5 | 28900455 | 28902518 | . |
| MSTRG.4225 | ENSG00000237283 | L1M4b | 1 | 0 | 1 | 1 | 188705183 | 188822360 | − |
| MSTRG.44653 | ENSG00000274525 | MSTD | 1 | 1 | 0 | 5 | 181281375 | 181297494 | + |
| | | (AluSq2) | | | | | | | |
| MSTRG.45033 | (Novel) | MLT1J1-int | 1 | 0 | 1 | 6 | 23311344 | 23609239 | − |
| MSTRG.48204 | ENSG00000189212 | L1M5 | 1 | 0 | 1 | 7 | 35079989 | 35186141 | − |
| | | (AluSc) | | | | | | | |
| MSTRG.49599 | ENSG00000225546 | THE1A | 1 | 0 | 1 | 7 | 119460150 | 119907375 | − |
| | | (L1PA16) | | | | | | | |
| MSTRG.54473 | ENSG00000228714 | L1PB4_1 | 1 | 0 | 1 | 9 | 115252538 | 115744330 | − |
| MSTRG.54479 | (Novel) | L1PB4_2 | 1 | 0 | 1 | 9 | 115640954 | 115641408 | − |
| MSTRG.5476 | ENSG00000229205 | L1MA6 | 1 | 1 | 0 | 10 | 1159768 | 1289445 | + |
| | | (MER4B) | | | | | | | |
| MSTRG.56887 | (Novel) | L2 | 1 | 1 | 0 | X | 105494118 | 105538265 | − |
| MSTRG.57724 | ENSG00000232348 | HERVE_a-int | 1 | 0 | 1 | Y | 8602037 | 8650498 | + |
| MSTRG.57727 | (Novel) | HERVK14C-int | 1 | 0 | 1 | Y | 8993071 | 9030576 | − |
| MSTRG.57807 | (Novel) | HERV17-int | 0 | 1 | 1 | Y | 19076163 | 19088030 | + |
| MSTRG.57886 | (Novel) | L1ME2 | 1 | 1 | 0 | Y | 56856798 | 56859188 | . |
| MSTRG.5851 | ENSG00000227932 | L1MC5 | 1 | 0 | 1 | 10 | 26639563 | 26697625 | − |
| MSTRG.8675 | ENSG00000255042 | L2b | 1 | 1 | 0 | 11 | 50279776 | 50298482 | − |
| | | (FLAM_A) | | | | | | | |

¹In these columns, “1” denotes a differentially expressed (DE) retrotransposon in this cohort having P < 0.1 in DESeq test, is highly expressed in the response group, and for which the log2 fold change in expression is larger than 2.5.
²Although TCGA-SKCM RNA-Seq didn't use stranded sequencing protocol, the strand information of novel transcriptions can still be determined based on splicing events. The non-spliced novel transcriptions have no strand information. Their strands were labeled as “.”.
“(Novel)” refers to retrotransposons identified by de novo transcript assembly as described herein. These retrotransposons are not represented in Ensembl(v92), hg38, or the TCGA data analyzed during the de novo transcript assembly.
³For some retrotransposons, a (bracketed retrotransposon) name is listed, this is as listed in Table 5 (same Gene ID, same chromosome position).

2.12.6. Determination of Correlation Between the Retrotransposon Expression Signature with Known Gene Expression Signatures

We further assessed the correlation of our retrotransposon-based signature with other established signatures predictive of response to checkpoint immunotherapy. Particularly, this was done for our signature of 30 and 54 retrotransposons, respectively (and similar results were obtained with the 29 and 50 retrotransposon signatures). Other established retrotransposon signatures include tumor mutation burden (substitution and indel load), immune cytolytic activity (CYT; Rooney et al. 2015, Cell 160:48-61), T cell-inflamed gene expression profile (GEP; Cristescu et al. 2018, Science 362:eaar3593), interferon-γ (INFγ; Ayers et al. 2017, J Clin Investig 127:2930-2940), type I/type II-interferon (INF; Hall et al. 2012, Proc Natl Acad Sci USA 109: 17609-17614), the immuno-predictive score IMPRES (Auslander et al. 2018, Nature Medicine 24:1545-1549), as well as the expression of PD1 and PD-L1 (Table 4). Interestingly, the correlation of both our signatures to each of these established signatures was consistently lower than 0.2, suggesting that our signatures independently predict overall survival after cancer immunotherapy.

This also suggests that our signatures can be used in combination with these established signatures to further enhance their predictive power. There was, however, a marginal but significant correlation with mutation burden and interferon expression (INFγ, type I/type II-INF), suggesting our biomarkers are favourably expressed in the immunity-active tumors, i.e., “hot” tumors. Therefore, our signatures of retrotransposons could also be used to classify hot versus cold tumors.

TABLE 4
Correlations of different signatures.

| | Sig30 | Sig54 | Substit | Indel | CYT | GEP | IFNγ |
|---|---|---|---|---|---|---|---|
| Sig30 | — | 0.91*** | 0.102* | 0.092* | 0.0516 | 0.0469 | 0.106* |
| Sig54 | 0.895*** | — | 0.142** | 0.136** | −0.00678 | −0.0119 | 0.0577 |
| Substi- | 0.0922* | 0.137** | — | 0.636*** | 0.051 | 0.0896 | 0.137** |
| tution | | | | | | | |
| Indel | 0.114* | 0.167*** | 0.685*** | — | −0.0475 | −0.0239 | 0.0299 |
| CYT | 0.0761 | 0.0153 | 0.0798 | −0.0474 | — | 0.959*** | 0.908*** |
| GEP | 0.0796 | 0.00993 | 0.0993* | −0.0419 | 0.956*** | — | 0.96*** |
| IFNγ | 0.13** | 0.076 | 0.135** | −0.00172 | 0.903*** | 0.957*** | — |
| Type | 0.155*** | 0.136** | 0.148** | 0.0162 | 0.543*** | 0.638*** | 0.737*** |
| I-IFN | | | | | | | |
| Type | 0.185*** | 0.135** | 0.135** | −0.00136 | 0.812*** | 0.87*** | 0.916*** |
| II-IFN | | | | | | | |
| IMPRES | −0.0896 | −0.119* | 0.0321 | 0.0103 | 0.446*** | 0.446*** | 0.383*** |
| PD1 | 0.0567 | −0.00299 | 0.0772 | −0.0406 | 0.947*** | 0.939*** | 0.876*** |
| PD-L1 | 0.086 | 0.0529 | 0.144** | 0.0222 | 0.783*** | 0.847*** | 0.86*** |

Spearman correlation

| | Type | Type | | | | |
|---|---|---|---|---|---|---|
| | I-IFN | II-IFN | IMPRES | PD1 | PD-L1 | |
| Sig30 | 0.139** | 0.166*** | −0.0997* | 0.028 | 0.0549 | Pearson |
| Sig54 | 0.128** | 0.124** | −0.117* | −0.0238 | 0.0321 | corre- |
| Substi- | 0.17*** | 0.154*** | −0.00664 | 0.035 | 0.163*** | lation |
| tution | | | | | | |
| Indel | 0.0701 | 0.0471 | 0.00775 | −0.0607 | 0.089 | |
| CYT | 0.521*** | 0.81*** | 0.416*** | 0.942*** | 0.756*** | |
| GEP | 0.621*** | 0.871*** | 0.424*** | 0.929*** | 0.823*** | |
| IFNγ | 0.716*** | 0.914*** | 0.365*** | 0.868*** | 0.82*** | |
| Type | — | 0.736*** | 0.161*** | 0.481*** | 0.639*** | |
| I-IFN | | | | | | |
| Type | 0.742*** | — | 0.287*** | 0.758*** | 0.758*** | |
| II-IFN | | | | | | |
| IMPRES | 0.16*** | 0.296*** | — | 0.413*** | 0.299*** | |
| PD1 | 0.507*** | 0.771*** | 0.462*** | — | 0.694*** | |
| PD-L1 | 0.711*** | 0.799*** | 0.309*** | 0.743*** | — | |

Spearman correlation

Pearson correlations and Spearman correlations were shown in yellow and cyan colors, respectively. For Sig30 and Sig54, we used the log 2(N+1) as signatures, where N is the number of expressed biomarkers among the 30 biomarkers from Leuven+Hugo data (Sig30), or among the 54 biomarkers from all three cohorts (Sig54).

“Sig30”: Signature of 30 retrotransposons from Leuven and Hugo patient cohorts. “Sig54”: Signature of 54 retrotransposons from Leuven, Hugo, and Riaz patient cohorts. “Substitution”: log 2 transferred substitution number=log 2(N+1), where N is the number of valid substitutions detected in the whole exon sequencing data (N+1 is used to avoid log(zero) problem). “Indel”: log 2 transferred indel number=log 2(N+1), where N is the number of valid indels (indel: insertion or deletion) detected in the whole exon sequencing data (+1 is to avoid log(zero) problem). “CYT”: immune cytolytic activity based on the expression of GZMA and PRF1. (Rooney et al. 2015, Cell 160:48-61). “GEP”: signature of T cell-inflamed gene expression profile (Cristescu et al. 2018, Science 362:eaar3593). “IFNγ”: signature of INF-γ (Ayers et al. 2017, J Clin Investig 127:2930-2940). “Type I-IFN”: gene IFIH1 and IFIH3 for type I interferon probe (Hall et al. 2012, Proc Natl Acad Sci USA 109: 17609-17614). “Type II-IFN”: gene GBP1 and GBP2 for type II interferon probe (Hall et al. 2012, Proc Natl Acad Sci USA 109: 17609-17614). “IMPRES”: immuno-predictive score (Auslander et al. 2018, Nature Medicine 24:1545-1549). “PD1”: PD1 expression (log 2 FPKM). “PD-L1”: PD-L1 expression (log 2 FPKM). FPKM is very similar to RPKM; whereas RPKM was designed for single-end RNA-seq (every read corresponded to a single sequenced fragment), FPKM was designed for paired-end RNA-seq. With paired-end RNA-seq, two reads can correspond to a single fragment, or, if one read in the pair did not map, one read can correspond to a single fragment. The only difference between RPKM and FPKM is that FPKM takes into account that two reads can map to one fragment (and so it doesn't count this fragment twice).

### Example 2.13. De Novo Transcript Detection in Samples of Melanoma Patients Treated with Immune Checkpoint Inhibitor, 2^{nd} Round

This Example mirrors Example 2.12 and describes results of a further analysis of de novo transcription. Terminology in this Example is identical to that of Example 2.12; however, reference is made to cryptic transcripts as a more general term for retrotransposons and IncRNAs (long non-coding RNAs) as in the further analysis some transcripts were identified that could not be clearly linked to a retrotransposon. In any case, the transcripts listed in any expression signature (“Sig”) all were identified as being differentially expressed (“differentially expressed transcripts”) between responders and non-responders to treatment with an immune checkpoint inhibitor.

2.13.1 Patient Cohorts: Are the Same as Described in Example 2.12.1.

2.13.2. De Novo Transcript Detection

We first de novo assembled sequencing reads obtained by RNA-sequencing on 472 publicly-available melanoma tumor samples in TCGA. To this end, we downloaded the aligned bam files from TCGA, and de novo assembled reads using StringTie v1.3.4 by applying the default parameter settings on each patient file, respectively. The known human gene annotation Ensembl92 were also inputted as guidance. All the outputted GTF files were further integrated using stringtie merge. After excluding protein-coding genes, a melanoma cryptic transcript annotation was built.

Next, we assessed RNA-seq data from tumors belonging to each of the 3 patient cohorts treated with checkpoint inhibitors. First, we preprocessed the fastq files by converting pair-end reads to single-end reads and chopping all the reads into 51 nucleotides. Then these data were aligned to the human genome (GRCh38) using STAR with the tolerance of two mismatches. At last, we called read numbers for cryptic transcripts based on the annotation build in TCGA and then calculated the number of Reads Per Kilobase of transcript, per Million mapped reads (RPKM), as a read-out of gene expression.

2.13.3. Differentially Expressed Transcripts in Leuven and Hugo Cohorts

First, we applied DESeq to identify all differentially expressed cryptic transcripts between patients displaying a treatment response (PR and CR) versus patients displaying no response (PD and SD) groups, in both cohorts from Leuven and Hugo et al. (Hugo et al. 2016, Cell 165:35-44), respectively (FIG. 19). After applying an expression level filter (FPKM>1 in any samples of both cohorts), we considered the cryptic transcripts with P<0.05 and a log 2 fold change >2.5 as differentially expressed (DE). Overall, we identified cryptic transcripts for which expression was high in response versus non-response patients, or vice versa. Of these, 24 cryptic transcripts were upregulated in patients with a treatment response in both cohorts, while only 1 cryptic transcript was lower in responding patients. This bias was significant (two-tailed Fisher exact test, p=2.98×10⁻⁶) (FIG. 20). The former 24 cryptic transcripts were considered as potential biomarkers, and the set of these 24 differentially expressed transcripts is referred to as the “Sig24” signature or panel, or as “Sig24” in short.

Next, we developed a prediction method to leverage these 24 biomarkers as a potential marker of treatment efficacy for checkpoint immunotherapy. First, a threshold was set that allowed us to determine when a given cryptic transcripts could be considered as ‘being expressed’. Particularly, we defined a cutoff of RPKM>0.5 to consider a cryptic transcript as expressed. This cutoff could separate reliable expressions from noises according to the RPKM distribution of the 24 cryptic transcripts in both cohorts (FIG. 21). Second, we established a cutoff to consider a tumor sample as a high- or low-expressor of cryptic transcripts. For this threshold, we counted the number of cryptic transcripts with a RPKM>0.5. According to the ROC curve (FIG. 22), the optimal threshold is 2 cryptic transcripts.

Therefore, we chose N=2 as the cut-off of expressed cryptic transcripts to consider a tumor sample as a high- or low-expressor of cryptic transcripts.

We tested the signature of 24 cryptic transcripts with its defined thresholds to predict overall survival (OS) in the Leuven and Hugo et al. (Hugo et al. 2016, Cell 165:35-44) melanoma cohorts (FIG. 23). In Hugo et al., significant OS differences between high- versus low-expressors of cryptic transcripts were observed (p<0.02; 32.2 versus 14.4 months). In the Leuven cohort, overall survival was longer in high-expressors of cryptic transcripts compared to low-expressors of cryptic transcripts, but effects were not significant (P=0.177; 110.2 versus 69.2 months), presumably due to the limited number of patients in this cohort and the limited follow-up available for some patients. When pooling both cohorts, survival differences predicted by the 24 cryptic transcripts gene signature were also significant (P=0.0315, 110.2 months versus 69.2 months). This significance is not compromised when corrected for patient gender and age factors (P=0.008).

2.13.4. Validation of Differentially Expressed Transcripts on Independent Riaz Cohort

In an effort to independently validate these observations, we applied the Sig24 signature at the same thresholds to a third publicly-available and independently established cohort of 25 melanoma patients receiving nivolumab, i.e., (Riaz et al. 2017, Cell 171:934-949). We analyzed differences on OS based on high- versus low-expressors of cryptic transcripts (FIG. 24) and observed a significant difference (p=0.0127; median survival: 27.7 months versus 15.5 months) in high versus low-expressors of cryptic transcripts. Additionally, we compared expression of these cryptic transcripts in paired pre-treatment versus on-treatment tumor samples (FIG. 25). A significant down-regulation of cryptic transcript expression was observed, and this only in patients with a response to checkpoint immunotherapy but not in those without a response (two-tailed Wilcoxon signed rank test, p<0.001). Tumor mutation burden is also established as a biomarker for checkpoint immunotherapy and the allelic frequency of mutation-carrying subclones is reduced during therapy, suggesting that similarly tumor clones expressing cryptic transcripts are under a negative selection bias during cancer immunotherapy.

2.13.5. Applying Differentially Expressed Transcripts as Biomarkers on all Three Patient Cohorts

Above results not only bring a useful signature to predict the immunotherapy outcomes in melanoma patients, but also showed the effectiveness of our methodology. In order to reinforce the signature using all three cohorts as a discovery set, rather than only two of them, we also performed DESeq tests for the response vs. non-response patients on different combinations. By applying the same criteria, we found 9 cryptic transcripts shared between Leuven and Riaz cohorts (referred to as the “Sig9b” signature or panel, or as “Sig9b” in short), and 4 cryptic transcripts shared between Hugo and Riaz cohorts (referred to as the “Sig4” signature or panel, or as “Sig4” in short) (FIG. 26). Expression of these 9 and 4 cryptic transcripts was higher in responding versus non-responding patients. Interestingly, by generating a 9 cryptic transcript- and 4 cryptic transcript-based signature, and by applying the same criteria to establish cut-offs for RPKM cutoff and number of cryptic transcripts expressed at RPKM>0.5, we could show that both signatures significantly predicted outcome in a pooled population (FIG. 27, panel II and III).

Overall, we identified 3 further signatures (Sig24, Sig9b, and Sig4) which predicted overall survival after anti-PD1 immunotherapy in melanoma. By merging these 3 signatures, a set of 33 unique cryptic transcript markers is obtained (referred to as “Sig33” signature or panel, or as “Sig33” in short). There coordinates within the human reference genome are listed in Table 5. For these unified 33 cryptic transcripts, we found N-cutoff=3 shows the best distinguishability on treatment responses through the similar ROC analysis (FIG. 28). Also, this unified signature shows significant prediction power on OS of all patients (p<0.01) (FIG. 27, panel IV).

TABLE 5
Cryptic transcripts biomarker coordinates in human genome GRCh38.
Transcript biomarkers in italics are not listed in Table 3.

| | | | | | | differently | | |
|---|---|---|---|---|---|---|---|---|
| | location in | | over-lapping | | max. | expressed | | |
| | genome | IncRNA | retro- | predictive | expression | in⁴ | | |
| Gene ID | (GRCh38)¹ | name | transposon² | accuracy³ | (RPKM) | L | H | R |
| MSTRG.44653 | chr5: 181281375- | (Novel) | AluSq2 | 70% | 4.89 | 1 | 1 | 0 |
| | 181297494: + | | (MSTD) | | | | | |
| MSTRG.42183 | chr5: 28585921- | AC008825.1 | MLT2B3 | 67% | 3.24 | 1 | 1 | 0 |
| | 28809249: − | | (MLT1E2) | | | | | |
| MSTRG.8675 | chr11: 50279776- | (Novel) | FLAM_A | 66% | 14.96 | 1 | 1 | 0 |
| | 50298482: − | | (L2b) | | | | | |
| MSTRG.34542 | chr22: 11066267- | CU104787.1 | IncRNA1 | 64% | 23.70 | 1 | 1 | 0 |
| | 11068382: + | | | | | | | |
| MSTRG.38237 | chr3: 145333834- | (Novel) | L1M1 | 64% | 8.96 | 1 | 1 | 0 |
| | 145381982: + | | (L1MD1) | | | | | |
| MSTRG.37337 | chr3: 80871322- | (Novel) | L1PREC2 | 64% | 1.70 | 1 | 1 | 0 |
| | 80876188: + | | | | | | | |
| MSTRG.19710 | chr16: 17831751- | (Novel) | AluSz | 64% | 1.64 | 1 | 1 | 0 |
| | 17868474: − | | (MIRb) | | | | | |
| MSTRG.24437 | chr18: 6727053- | (Novel) | MIRb | 63% | 4.00 | 0 | 1 | 1 |
| | 6728981: + | | | | | | | |
| MSTRG.56476 | chrX: 71760764- | (Novel) | MIRb | 61% | 16.04 | 1 | 1 | 1 |
| | 71763075: − | | | | | | | |
| MSTRG.40292 | chr4: 79314234- | (Novel) | L1MC2 | 61% | 10.84 | 1 | 1 | 0 |
| | 79314447: . | | | | | | | |
| MSTRG.42146 | chr5: 25087450- | LINC02228 | MLT2C1 | 61% | 8.71 | 1 | 1 | 0 |
| | 25190956: − | | | | | | | |
| MSTRG.32532 | chr20: 6321979- | CASC20 | L2a | 61% | 3.12 | 1 | 1 | 0 |
| | 6585670: + | | (L1ME4a) | | | | | |
| MSTRG.56743 | chrX: 99372197- | (Novel) | LTR12C | 61% | 2.95 | 1 | 1 | 0 |
| | 99372745: + | | | | | | | |
| MSTRG.11332 | chr12: 18041309- | (Novel) | AluY | 61% | 2.69 | 1 | 1 | 1 |
| | 18046380: − | | (HERV9-int) | | | | | |
| MSTRG.19711 | chr16: 17849826- | (Novel) | MLT2E | 61% | 2.09 | 1 | 1 | 0 |
| | 17854608: + | | (L1PA17) | | | | | |
| MSTRG.56745 | chrX: 99705863- | (Novel) | AmnSINE1 | 61% | 1.93 | 1 | 1 | 0 |
| | 99712287: + | | | | | | | |
| MSTRG.56471 | chrX: 71662998- | (Novel) | IncRNA2 | 60% | 6.26 | 1 | 1 | 0 |
| | 71667807: − | | | | | | | |
| MSTRG.41790 | chr5: 3336447- | LINC01019 | MLT1C | 60% | 5.86 | 1 | 1 | 0 |
| | 3536094: − | | | | | | | |
| MSTRG.54479 | chr9: 115640954- | (Novel) | L1PB4 | 60% | 4.74 | 1 | 0 | 1 |
| | 115641408: − | | (L1PB4_2) | | | | | |
| MSTRG.41114 | chr4: 147358266- | (Novel) | THE1C | 60% | 1.53 | 1 | 1 | 0 |
| | 147359552: − | | | | | | | |
| MSTRG.42080 | chr5: 19172546- | (Novel) | LTR12C | 60% | 1.42 | 0 | 1 | 1 |
| | 19288246: − | | | | | | | |
| MSTRG.14514 | chr13: 68025900- | (Novel) | MLT1G3 | 60% | 1.38 | 1 | 0 | 1 |
| | 68037147: + | | | | | | | |
| MSTRG.5476 | chr10: 1159768- | LINC00200 | MER4B | 59% | 11.57 | 1 | 1 | 0 |
| | 1289445: + | | (L1MA6) | | | | | |
| MSTRG.24775 | chr18: 30095986- | (Novel) | LTR1A2 | 59% | 5.76 | 1 | 1 | 0 |
| | 30176042: − | | | | | | | |
| MSTRG.41489 | chr4: 178312070- | (Novel) | L1MC3 | 59% | 3.94 | 1 | 0 | 1 |
| | 178313749: . | | | | | | | |
| MSTRG.13707 | chr13: 18331933- | (Novel) | IncRNA3 | 59% | 3.70 | 1 | 1 | 0 |
| | 18348596: + | | | | | | | |
| MSTRG.13742 | chr13: 19181981- | (Novel) | MSTA-int | 57% | 11.66 | 1 | 1 | 0 |
| | 19189771: + | | (MSTA) | | | | | |
| MSTRG.49599 | chr7: 119460150- | LINC02476 | L1PA16 | 57% | 6.98 | 1 | 0 | 1 |
| | 119907375: − | | (THE1A) | | | | | |
| MSTRG.14671 | chr13: 83906092- | (Novel) | MER61A | 57% | 6.12 | 1 | 1 | 0 |
| | 84069354: + | | (MER61A-int) | | | | | |
| MSTRG.5851 | chr10: 26639563- | (Novel) | L1MC5 | 57% | 4.28 | 1 | 0 | 1 |
| | 26697625: − | | | | | | | |
| MSTRG.5485 | chr10: 2527426- | (Novel) | AluSx1 | 57% | 1.84 | 1 | 0 | 1 |
| | 2529001: . | | | | | | | |
| MSTRG.25244 | chr18: 66114175- | (Novel) | AluYe5 | 56% | 3.62 | 1 | 1 | 0 |
| | 66258594: − | | (THE1b) | | | | | |
| MSTRG.48204 | chr7: 35079989- | (Novel) | AluSc | 54% | 14.54 | 1 | 0 | 1 |
| | 35186141: − | | (L1M5) | | | | | |

L: Leuven cohort;
H: Hugo cohort;
R: Riaz cohort
¹Although TCGA-SKCM RNA-Seq didn't use stranded sequencing protocol, the strand information of novel transcriptions can still be determined based on splicing events. The non-spliced novel transcriptions have no strand information. Their strands were labeled as “.”.
²If a cryptic transcript overlaps more than one retrotransposons, the one with the longest overlap region is listed. For some a (bracketed retrotransposon) name is listed, this is as listed in Table 3 (same Gene ID, same chromosome position). IncRNA1, IncRNA2, IncRNA3: no retrotransposon annotation, fictitious names.
³Calculated by the rate of true-positive + true negative predicted by a single cryptic transcription. RPKM cutoff is 0.5.
⁴In these columns, “1” denotes a cryptic transcripts in this cohort has P < 0.1 in DESeq test, highly expressed in the response group, and log2 fold change is larger than 2.5.

2.13.6. Independence of Differentially Expressed Transcript Signatures with Known Gene Expression Signatures

We further assessed the correlation of the above cryptic-transcript-based signature with other established signatures predictive of response to checkpoint immunotherapy. Particularly, this was done for the signatures of 24 (Sig24) and 33 cryptic transcripts (Sig33), respectively. Other established cryptic transcript signatures include tumor mutation burden (substitution and indel load), immune cytolytic activity (CYT), T cell-inflamed gene expression profile (GEP), interferon-γ (INFγ), type I/type II-interferon (INF), the immuno-predictive score IMPRES, as well as the expression of PD1 and PD-L1 (Table 6). Interestingly, the correlation of both the Sig24 and Sig33 signature to each of these established signatures was consistently lower than 0.2, suggesting that these signatures independently predict overall survival after cancer immunotherapy. This also suggests that these signatures can be used in combination with these established signatures to further enhance their predictive power. There was, however, a correlation with interferon expression (INFγ, type I/type II-INF), suggesting the herein described biomarkers are favorably expressed in immune-active tumors, i.e., “hot” tumors. Therefore, the signatures of cryptic transcripts could also be used to classify hot versus cold tumors.

TABLE 6
Correlations of different signatures.

| | | | | | | | Type | Type | | | | |
|---|---|---|---|---|---|---|---|---|---|---|---|---|
| | Sig24 | Sig33 | TMB | CYT | GEP | IFNγ | I-IFN | II-IFN | IMPRES | PD1 | PD-L1 | |
| Sig24 | — | 0.995*** | 0.0822 | 0.0458 | 0.0311 | 0.0845 | 0.0818 | 0.107* | 0.0151 | −0.0307 | 0.0477 | Pear- |
| Sig33 | 0.957*** | — | 0.0705 | 0.0294 | 0.0144 | 0.0699 | 0.0737 | 0.099* | 0.00342 | −0.0391 | 0.0336 | son |
| TMB | 0.0794 | 0.0821 | — | 0.0368 | 0.0885 | 0.139** | 0.158** | 0.157** | −0.05651 | 0.0569 | 0.0229 | corre- |
| CYT | 0.0664 | 0.0156 | 0.0632 | — | 0.959*** | 0.908*** | 0.516*** | 0.811*** | 0.817*** | 0.744*** | 0.934*** | lation |
| GEP | 0.0636 | 0.0132 | 0.095 | 0.956*** | — | 0.96*** | 0.617*** | 0.872*** | 0.85*** | 0.751*** | 0.931*** | |
| IFNγ | 0.114* | 0.0678 | 0.14** | 0.903*** | 0.957*** | — | 0.711*** | 0.914*** | 0.779*** | 0.68*** | 0.89*** | |
| Type | 0.122** | 0.0996* | 0.146** | 0.537*** | 0.633*** | 0.732*** | — | 0.733*** | 0.445*** | 0.364*** | 0.555*** | |
| I-IFN | | | | | | | | | | | | |
| Type | 0.152*** | 0.112* | 0.125* | 0.813*** | 0.87*** | 0.916*** | 0.738*** | — | 0.691*** | 0.591*** | 0.789*** | |
| II-IFN | | | | | | | | | | | | |
| IMPRES | 0.056 | 0.0127 | −0.0438 | 0.826*** | 0.863*** | 0.796*** | 0.481*** | 0.712*** | — | 0.832*** | 0.839*** | |
| PD1 | −0.0155 | −0.0438 | 0.0873 | 0.749*** | 0.751*** | 0.682*** | 0.391*** | 0.606*** | 0.793*** | — | 0.753*** | |
| PD-1 | 0.0841 | 0.0463 | 0.0349 | 0.917*** | 0.918*** | 0.897*** | 0.574*** | 0.786*** | 0.828*** | 0.727*** | — | |

Spearman correlation

Pearson correlations and Spearman correlations were shown in yellow and cyan colors, respectively. For Sig24 and Sig33, we used the log 2(N+1) as signatures, where N is the number of expressed biomarkers among the 24 biomarkers from Leuven+Hugo data (Sig24), or among the 33 biomarkers from all three cohorts (Sig33). Significance: *-p<0.05, **-p<0.01, ***-p<0.001.

“Sig29”: Signature of 29 retrotransposons from Leuven and Hugo patient cohorts. “Sig 33”: Signature of 33 retrotransposons from Leuven, Hugo and Riaz patient cohorts. “TMB”: tumor mutation burden, log 2-transferred tumor mutation load.

“CYT”: immune cytolytic activity based on the expression of GZMA and PRF1. (Rooney et al. 2015, Cell 160:48-61). “GEP”: signature of T cell-inflamed gene expression profile (Cristescu et al. 2018, Science 362:eaar3593). “IFNγ”: signature of INF-γ (Ayers et al. 2017, J Clin Investig 127:2930-2940). “Type I-IFN”: gene IFIH1 and IFIH3 for type I interferon probe (Hall et al. 2012, Proc Natl Acad Sci USA 109: 17609-17614). “Type II-IFN”: gene GBP1 and GBP2 for type II interferon probe (Hall et al. 2012, Proc Natl Acad Sci USA 109: 17609-17614). “IMPRES”: immuno-predictive score (Auslander et al. 2018, Nature Medicine 24:1545-1549). “PD1”: PD1 expression (log 2 FPKM). “PD-L1”: PD-L1 expression (log 2 FPKM). FPKM is very similar to RPKM; whereas RPKM was designed for single-end RNA-seq (every read corresponded to a single sequenced fragment), FPKM was designed for paired-end RNA-seq. With paired-end RNA-seq, two reads can correspond to a single fragment, or, if one read in the pair did not map, one read can correspond to a single fragment. The only difference between RPKM and FPKM is that FPKM takes into account that two reads can map to one fragment (and so it doesn't count this fragment twice).

1. A method of detecting susceptibility to immunotherapy or immunogenic therapy of a tumor in a subject prior to or early after start of the immunotherapy or of the immunogenic therapy, the method comprising:
detecting an increase in the expression level of at least one retrotransposon in a sample obtained from the subject, and wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein the increased expression level of the retrotransposon in the sample is relative to the expression level of the same retrotransposon in a control sample or compared to a standard value.

2. A method of measuring tumor response to immunotherapy or to immunogenic therapy in a subject, the method comprising: the step of
detecting an decrease in the expression level of at least one retrotransposon in a sample obtained from the subject, and wherein the retrotransposon is selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5; and wherein the decrease in the expression level of the retrotransposon in the sample relative to the expression level of the same retrotransposon in a sample obtained from the subject prior to immunotherapy or immunogenic therapy or in a sample obtained at an earlier time-point during immunotherapy or immunogenic therapy.

3. The method according to claim 1, wherein the method comprises detecting the expression level of at least 4 retrotransposons in the sample obtained from the subject, and wherein the retrotransposons are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5.

4. The method according to claim 1, wherein the method comprises detecting the expression level of at least 4 retrotransposons in the sample obtained from the subject, and wherein the retrotransposons are selected from the retrotransposons HERV9-int/AluY (chr12), L1M4 (chr13), MSTA/MSTA-int (chr13), MLT1G3 (chr13), MER57E1 (chr13), MER61-int/MER61A (chr13), L1PB3 (chr13), AluSx3 (chr14), L1ME3Cz (chr14), L1MC4a (chr14), LTR16C (chr14), MIRb/AluSz (chr16), L1PA17/MLT2E (chr16), MamGypsy2-I (chr16), L1PA5 (chr18), LTR1A2 (chr18), THE1A (chr18), THE1B/AluYe5 (chr18), MIRb (chr2), THE1C (chr2), L1ME4a/L2a (chr20), LTR67B (chr20), L1MCa (chr21), L4_A_Mam (chr22), MLT1A1 (chr22), PRIMA41-int (chr22), ERVL-B4-int (chr3), L1PREC2 (chr3), L1MD1/L1M1 (chr3), L2a (chr3), THE1D (chr4), THE1B (chr4), L1MC3 (chr4), MLT2C1 (chr5), L1PREC2 (chr5), L1MB8 (chr5), MLT1E2/MLT2B3 (chr5), L1MB2 (chr5), L1MB4 (chr5), L1M4b (chr1), MSTD/AluSq2 (chr5), MLT1J1-int (chr6), L1M5/AluSc (chr7), THE1A/L1PA16 (chr7), L1PB4_1 (chr9), L1PB4_2 (chr9), L1MA6/MER4B (chr10), L2 (chrX), L1MC5 (chr10), and L2b/FLAM_A (chr11), all as defined in Table 3; and/or further selected from the retrotransposons IncRNA1 (chr22), MIRb (chr18), MIRb (chrX), L1MC2 (chr4), LTR12C (chrX), AmnSINE1 (chrX), IncRNA2 (chrX), MLT1C (chr5), THE1C (chr4), LTR12C (chr5), IncRNA3 (chr13), AluSx1 (chr10), all as defined in Table 5.

5. The method according to claim 1, further including comprising detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES).

6. The method according to claim 5, wherein the markers of tumor mutational burden are chosen from substitution markers, indel markers, and microsatellite instability markers.

7. The method according to claim 1, wherein the tumor is melanoma.

8. The method according to claim 1, further comprising administering a therapeutically effective amount of the immunotherapeutic or immunogenic agent to the subject.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The method according to claim 2, further comprising detecting the status of one or more further diagnostic markers or biomarkers selected from immune checkpoint gene expression, markers of tumor mutational burden, T cell-inflamed gene expression, immune cytolytic activity, interferon-related gene expression, expression of hypoxia marker genes, hypoxia-dependent methylation of promoters of tumor suppressor genes, expression of innate anti-PD-1 resistance genes, immune cell composition, immune-predictive score (IMPRES), expression of anti-PD-1 resistance genes (IPRES).

21. The method according to claim 20, wherein the markers of tumor mutational burden are chosen from substitution markers, indel markers, and microsatellite instability markers.

22. The method according to claim 2, wherein the tumor is melanoma.


SEQUENCE LISTING (12 sequences)

SEQ ID NO: 1 - DNA, 29 bp, Artificial Sequence
  Primer
aggtgcaattgttcctccgcctcccttac

SEQ ID NO: 2 - DNA, 31 bp, Artificial Sequence
  Primer
aagggcaattgccgagctttttcctttacga

SEQ ID NO: 3 - DNA, 22 bp, Artificial Sequence
  Primer
gtttgggttagtgatagggtgt

SEQ ID NO: 4 - DNA, 24 bp, Artificial Sequence
  Primer
aaaccctcccttctactcctttcc

SEQ ID NO: 5 - DNA, 20 bp, Artificial Sequence
  Primer
tcgtttccgacttttccatc

SEQ ID NO: 6 - DNA, 19 bp, Artificial Sequence
  Primer
cagccagaatgttggcaat

SEQ ID NO: 7 - DNA, 22 bp, Artificial Sequence
  Primer
cacttgctgaataattgggtgt

SEQ ID NO: 8 - DNA, 22 bp, Artificial Sequence
  Primer
ctgttgtccagttttcttcacg

SEQ ID NO: 9 - DNA, 24 bp, Artificial Sequence
  gRNA
caccgtgaaatagaacggcggcga

SEQ ID NO: 10 - DNA, 24 bp, Artificial Sequence
  gRNA
aaactcgccgccgttctatttcac

SEQ ID NO: 11 - DNA, 25 bp, Artificial Sequence
  gRNA
caccgcactaccagagctaactcag

SEQ ID NO: 12 - DNA, 25 bp, Artificial Sequence
  gRNA
aaacctgagttagctctggtagtgc